(19) United States
(12) Patent Application Publication
(10) Pub. No.: US 2010/0143379 A1
(43) Pub. Date: Jun. 10, 2010

(54) MIF AGONISTS AND ANTAGONIST AND THERAPEUTIC USES THEREOF

(76) Inventors: Richard Bucala, Cos Cob, CT (US); Lin Leng, New York, NY (US); Sarah Doernberg, San Francisco, CA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 11/919,307

(22) PCT Filed: Apr. 26, 2006
(86) PCT No.: PCT/US2006/016254

§ 371 Date: Feb. 17, 2010

Related U.S. Application Data
(60) Provisional application No. 60/675,303, filed on Apr. 26, 2005.
(60) Provisional application No. 60/685,533, filed on May 27, 2005.
(60) Provisional application No. 60/687,481, filed on Jun. 2, 2005.
(60) Provisional application No. 60/688,833, filed on Jun. 9, 2005.
(60) Provisional application No. 60/688,865, filed on Jun. 8, 2005.
(60) Provisional application No. 60/725,049, filed on Oct. 7, 2005.
(60) Provisional application No. 60/732,797, filed on Nov. 2, 2005.

(51) Int. Cl. A61K 38/22; C12Q 1/68; C07H 21/04; C40B 40/06; A61K 31/7088; A61K 39/395; G01N 33/53; A61P 7/06; A61P 31/00

(52) U.S. Cl. 424/172.1; 435/6; 536/24.32; 506/16; 514/44 A; 514/8; 436/501

(57) ABSTRACT

The present invention relates to methods and compositions for selecting a subject for treatment with an agonist or antagonist of macrophage migration inhibitory factor (MIF), identifying a subject at risk for developing a disease associated with high or low MIF expression, predicting the severity of a disease associated with high or low MIF expression in a subject, and for predicting whether a subject is susceptible to a disease associated with high or low MIF expression. The invention also provides novel methods of diagnosing a patient for a disease associated with high or low MIF expression. Also provided are methods for treating a subject having a disease or disorder associated with high or low MIF expression.

### CROSS-REFERENCE TO RELATED APPLICATIONS

This application claims the benefit of U.S. Provisional Application No. 60/675,303, filed Apr. 26, 2005; U.S. Provisional Application No. 60/685,533, filed May 27, 2005; U.S. Provisional Application No. 60/687,481, filed Jun. 2, 2005; U.S. Provisional Application No. 60/688,865, filed Jun. 8, 2005; U.S. Provisional Application No. 60/698,833, filed Jul. 12, 2005; U.S. Provisional Application No. 60/725,049, filed Oct. 7, 2005; and U.S. Provisional Application No. 60/732,797, filed Nov. 2, 2005. The teachings of each of these referenced provisional applications are incorporated by reference herein in their entirety.

### STATEMENT REGARDING FEDERALLY-SPONSORED RESEARCH OR DEVELOPMENT

The invention described herein was supported, in whole or in part, by the National Institute of Health Grant Nos. AI042310, AI51306, AR49610, and AR50498. The United States government has certain rights in the invention.

### BACKGROUND OF THE INVENTION

Macrophage migration inhibitory factor (MIF) is a critical regulator of the innate and adaptive immune response. MIF is encoded by a unique polymorphic gene, and crystallization studies have shown MIF to define a new protein fold and structural superfamily. Despite the fact that the biological activity attributed to MIF first was described almost 30 years ago, information regarding MIF's precise role in cell physiology and immunity has emerged only recently.

Macrophage migration inhibitory factor (MIF) is a pleiotropic multifunctional cytokine with a mostly proinflammatory spectrum of action in the host immune response. MIF promotes the production of TNFα and other inflammatory mediators in an autocrine-paracrine fashion, and mice genetically-deficient in MIF are known to have a reduced inflammatory cytokine response by cells of the monocyte/macrophage lineage (Morand (2005). Intern. Med. J. 35:419-426; Gregory et al. (2004). Arthritis Rheum. 50:3023-3034; and Ichiyama et al. (2004). Cytokine 26:187-194). MIF levels have been reported to be increased in infectious and autoimmune diseases and have been reported to correlate with the severity of septic shock, rheumatoid arthritis, systemic sarcoidosis and inflammatory bowel disease (Amoli et al. (2002). J. Rheumatol. 29:1671-1673 and Leech et al. (1999). Arthritis Rheum. 42:1601-1608). It has been reported that anti-MIF monoclonal antibodies can prevent septic shock in mice (Calandra et al. Nat Med 2000; 6:164-70 and Lolis et al. (2003). Expert Opin. Ther. Targets. 7:153-164) and inhibit joint destruction in mouse models of inflammatory arthritis (Mikulowska et al. (1997). J. Immunol. 158:5514-5517). MIF also antagonizes the action of glucocorticoids (Calandra et al. (1995). Nature 377:68-71 and Calandra et al. (2003). Nat Rev Immunol 3:791-800), upregulates Toll-like receptor 4 (TLR-4) expression (Roger et al. (2001). Nature 414:920-924), controls Jab1 transcriptional effects (Kleemann et al. (2000). Nature 408:211-216), and suppresses activation-induced, p53-dependent apoptosis (Hudson et al. (1999). J Exp Med 190:1375-1382; Mitchell et al. (2002). Proc Natl Acad Sci USA 99:345-350; and Nguyen et al. (2003). J Immunol 170:3337-3347).

The Type II transmembrane protein, CD74, binds to MIF with high-affinity and is important for MIF biological activity (Leng et al. (2003). J Exp Med 197:1467-1476). MIF binds to the extracellular domain of CD74, and CD74 is required for MIF-induced activation of the extracellular signal-regulated kinase-1/2 MAP kinase cascade, cell proliferation, and PGE2 production. A recombinant, soluble form of CD74 binds MIF with a dissociation constant of approximately 9×10⁻⁹ Kd, as defined by surface plasmon resonance (BIAcore analysis), and soluble CD74 inhibits MIF-mediated extracellular signal-regulated kinase activation in defined cell systems. These identify MIF as a natural ligand for CD74, which has been implicated previously in signaling and accessory functions for immune cell activation.

Polymorphisms in cytokine genes may influence the severity of diseases in which the host inflammatory response plays a key role (McGuire et al. (1994). Nature 371:508-510; Pawlik et al. (2005). Scand. J. Rheumatol. 34:109-113; Cantor et al. (2005). Am. J. Gastroenterol. 100:1134-1142; and Wilson et al. (2005). J. Infect. Dis. 191:1705-1709). Recently, a search for DNA polymorphisms in human MIF revealed variants in the structure of the promoter region that affect the level of MIF expression (Baugh et al. (2002) Genes Immun. 3:170-176 and De Benedetti et al. (2003) Arthritis & Rheum 48:1398-1407). A tetranucleotide CATT repeat at position −794 regulates MIF transcriptional activity and subsequent protein production, with the number of repeats (5, 6, 7, or 8) proportional to the level of transcription in in vitro assays (Baugh et al. (2002) Genes Immun. 3:170-176). Individuals with greater number of CATT repeats (e.t., 6, 7, or 8) express more MIF. A single nucleotide polymorphism (SNP), G to C switch at position −173 of the MIF promoter, has also been found and may exert similar effects either by linkage dysequilibrium or by interaction with the CATT repeat (Donn et al. (2001). Arthritis Rheum 44:1782-5 and De Benedetti et al. (2003) Arthritis & Rheum 48

### SUMMARY OF THE INVENTION

The invention relates to novel uses for MIF agonists and MIF antagonists, and to novel types of MIF agonists and MIF antagonists. Further, the invention relates to methods of prognosis and diagonisis involving determining the MIF genotype of a subject.

In one aspect, the invention relates to a method of selecting a subject for treatment with a MIF antagonist, wherein the subject has a disease associated with high MIF expression or is at risk of developing a disease associated with high MIF expression, comprising genotyping the subject for the presence of a polymorphism associated with MIF expression, wherein a subject having a polymorphism associated with high MIF expression is selected for treatment with a MIF antagonist. In one embodiment, the disease associated with high MIF expression is a disease caused by a protozoan. In another embodiment, the disease associated with high MIF expression is malaria. In another embodiment, the disease associated with high MIF expression is anemia of chronic disease. In another embodiment, the disease associated with high MIF expression is asthma.

In another aspect, the invention also relates to a method of selecting a subject for treatment with a MIF agonist, wherein the subject has a disease associated with low MIF expression or is at risk of developing a disease associated with low MIF expression, comprising genotyping the subject for the presence of a polymorphism associated with MIF expression, wherein a subject having a polymorphism associated with low MIF expression is selected for treatment with a MIF agonist. In one embodiment, the disease associated with low MIF expression is an infection. In one embodiment, the infection leads to respiratory disease. In another embodiment, the disease associated with low MIF expression is pneumonia. In another embodiment, the disease associated with low MIF expression is Community Acquired Pneumonia (CAP). In another embodiment, the disease associated with low MIF expression is meningitis. In another embodiment, the disease associated with low MIF expression is influenza. In another embodiment, the disease associated with low MIF expression is sepsis. In another embodiment, the disease associated with low MIF expression is HIV infection. In another embodiment, the disease associated with low MIF expression is infection with a virus or another pathogen that uses CCR5 as a receptor.

In another aspect, the invention relates to a method of identifying a subject at risk of developing a disease associated with high MIF expression, comprising genotyping the subject for the presence of a polymorphism associated with MIF expression, wherein a subject having a polymorphism associated with high MIF expression is at a higher risk of developing a disease or disorder associated with high MIF expression than a subject having a polymorphism associated with low MIF expression. In one embodiment, the disease associated with high MIF expression is a disease caused by a protozoan. In another embodiment, the disease associated with high MIF expression is malaria. In another embodiment, the disease associated with high MIF expression is anemia of chronic disease.

In another aspect, the invention relates to a method of identifying a subject at risk of developing a disease associated with low MIF expression, comprising genotyping the subject for the presence of a polymorphism associated with MIF expression, wherein a subject having a polymorphism associated with low MIF expression is at a higher risk of developing a disease or disorder associated with low MIF expression than a subject having a polymorphism associated with high MIF expression. In one embodiment, the disease associated with low MIF expression is an infection. In one embodiment, the infection leads to respiratory disease. In another embodiment, the disease associated with low MIF expression is pneumonia. In another embodiment, the disease associated with low MIF expression is Community Acquired Pneumonia (CAP). In another embodiment, the disease associated with low MIF expression is meningitis. In another embodiment, the disease associated with low MIF expression is influenza. In another embodiment, the disease associated with low MIF expression is sepsis. In another embodiment, the disease associated with low MIF expression is HIV infection. In another embodiment, the disease associated with low MIF expression is infection with a virus or another pathogen that uses CCR5 as a receptor.

In another aspect, the invention relates to a method of predicting the severity of a disease associated with high MIF expression in a subject, comprising genotyping the subject for the presence of a polymorphism associated with MIF expression, wherein a subject having a polymorphism associated with high MIF expression is at a higher risk of developing a more severe disease than a subject having a polymorphism associated with low MIF expression. In one embodiment, the disease associated with high MIF expression is a disease caused by a protozoan. In another embodiment, the disease associated with high MIF expression is malaria. In another embodiment, the disease associated with high MIF expression is anemia of chronic disease. In another embodiment, the disease associated with high MIF expression is asthma.

In another aspect, the invention relates to a method of predicting the severity of a disease associated with low MIF expression in a subject, comprising genotyping the subject for the presence of a polymorphism associated with MIF expression, wherein a subject having a polymorphism associated with low MIF expression is at a higher risk of developing a more severe disease than a subject having a polymorphism associated with high MIF expression. In one embodiment, the disease associated with low MIF expression is an infection. In one embodiment, the infection leads to respiratory disease. In another embodiment, the disease associated with low MIF expression is pneumonia. In another embodiment, the disease associated with low MIF expression is Community Acquired Pneumonia (CAP). In another embodiment, the disease associated with low MIF expression is meningitis. In another embodiment, the disease associated with low MIF expression is influenza. In another embodiment, the disease associated with low MIF expression is sepsis. In another embodiment, the disease associated with low MIF expression is HIV infection. In another embodiment, the disease associated with low MIF expression is infection with a virus or another pathogen that uses CCR5 as a receptor.

In another aspect, the invention relates to a method of predicting whether a subject is susceptible to a disease associated with low MIF expression, comprising genotyping a subject for the presence of a polymorphism associated with MIF expression, wherein a subject having a polymorphism associated with low MIF expression is more susceptible to the disease than a subject having a polymorphism associated with high MIF expression. In one embodiment, the disease associated with low MIF expression is an infection. In one embodiment, the infection leads to respiratory disease. In another embodiment, the disease associated with low MIF expression is pneumonia. In another embodiment, the disease associated with low MIF expression is Community Acquired Pneumonia (CAP). In another embodiment, the disease associated with low MIF expression is meningitis. In another embodiment, the disease associated with low MIF expression is influenza. In another embodiment, the disease associated with low MIF expression is sepsis. In another embodiment, the disease associated with low MIF expression is HIV infection. In another embodiment, the disease associated with low MIF expression is infection with a virus or another pathogen that uses CCR5 as a receptor.

In another aspect, the invention relates to a method of predicting whether a subject is susceptible to a disease associated with high MIF expression, comprising genotyping a subject for the presence of a polymorphism associated with MIF expression, wherein a subject having a polymorphism associated with high MIF expression is more susceptible to the disease than a subject having a polymorphism associated with low MIF expression. In one embodiment, the disease associated with high MIF expression is a disease caused by a protozoan. In another embodiment, the disease associated with high MIF expression is malaria. In another embodiment, the disease associated with high MIF expression is anemia of chronic disease.

In any of the above described methods, the polymorphism associated with MIF expression may be selected from the group consisting of: (a) the presence of five, six, seven or eight CATT repeats in the −794 region of the MIF promoter, and (b) the presence of guanine or cytosine at position −173 of the MIF promoter. A subject having a polymorphism associated with high MIF expression is a subject having a C at position −173 in at least one of the two alleles of the MIF gene, or having six or more CATT repeats in at least one of the two alleles of the MIF gene. In a preferred embodiment, a subject having a polymorphism associated with high MIF expression is a subject: (a) having six or more CATT repeats in the −794 region of the MIF promoter in each of the two alleles of the MIF gene, or (b) having six or more CATT repeats in the −794 region of the MIF promoter in one allele and having a C at position −173 in each of the two alleles of the MIF gene. A subject having a polymorphism associated with low MIF expression is a subject having a G at position −173 of each of the two alleles of the MIF gene and having five CATT repeats in the −794 region of each of the two alleles of the MIF gene.

In any of the above described methods, any genotyping method can be used. In one embodiment, genotyping the subject for the presence of a polymorphism associated with MIF expression comprises: (a) contacting a sample obtained from the subject with a polynucleotide probe that hybridizes specifically to a polymorphism associated with high or low MIF expression; and (b) determining whether hybridization occurs, wherein hybridization indicates whether the subject comprises a polymorphism associated with high MIF expression or a polymorphism associated with low MIF expression, thereby genotyping the subject for the presence of a polymorphism associated with MIF expression. This genotyping method may further comprise: (c) contacting the sample with a control polynucleotide probe, wherein the control polynucleotide probe does not hybridize specifically to a polymorphism associated with MIF expression, and wherein hybridization of the polynucleotide probe but not the control polynucleotide probe indicates the presence of a MIF polymorphism associated with MIF expression.

In another embodiment, genotyping the subject for the presence of a polymorphism associated with MIF expression comprises: (a) contacting a sample obtained from the subject with a pair of amplifications primers, wherein said primers are capable of amplifying a portion of the MIF promoter comprising a polymorphism associated with MIF expression; (b) amplifying DNA in the sample, thereby producing amplified DNA; and (c) determining whether the amplified DNA comprises a polymorphism associated with high MIF expression or a polymorphism associated with low MIF expression, thereby genotyping the subject for the presence of a polymorphism associated with MIF expression. In one embodiment, the determining step comprises sequencing the amplified DNA. In another embodiment, the determining step comprises determining whether the sample hybridizes specifically to a polynucleotide probe that is specific for a polymorphism associated with high MIF expression or to a polynucleotide probe that is specific for a polymorphism associated with low MIF expression.

In another aspect, the invention provides a novel solid substrate for simultaneously genotyping a microsatellite repeat and a SNP, comprising at least two polynucleotide probes that are complementary to one or more polymorphic regions of the MIF gene wherein at least one of the probes detects a microsatellite repeat and at least one of the probes detects a SNP.

In one embodiment, at least one of the probes in the solid substrate hybridizes specifically to a guanine or a cytosine in the −173 region of the MIF promoter. In another embodiment, at least one of the probes in the solid substrate comprises a sequence selected from the group consisting of SEQ ID NO: 1, SEQ ID NO: 2, SEQ ID NO: 3 and SEQ ID NO: 4. In another embodiment, the solid support comprises: (a) a probe hybridizing specifically to SEQ ID NO: 1; (b) a probe hybridizing specifically to SEQ ID NO: 2; (c) a probe hybridizing specifically to SEQ ID NO: 3; (d) a probe hybridizing specifically to SEQ ID NO: 4; (e) a probe hybridizing specifically to guanine in the −173 region of the MIF promoter; and (f) a probe hybridizing specifically to cytosine in the −173 region of the MIF promoter. The solid support may be a chip or a microarray. In one embodiment the solid substrate is a thin film chip or microarray which permits the visual detection of a nucleic acid targets (indicating the presence/absence of a MIF polymorphism associated with high or low MIF expression) in the solid substrate with the unaided eye.

In one embodiment, the invention comprises a method of determining the MIF genotype of a subject, comprising: (a) contacting a solid substrate as described above for simultaneously genotyping a microsatellite repeat and a SNP with a sample obtained from a subject; and (b) determining whether the subject comprises a polymorphism associated with high MIF expression or whether the subject comprises a polymorphism associated with low MIF expression, thereby determining the MIF genotype of the subject.

In another embodiment, the invention comprises a method of determining the MIF genotype of a subject, comprising: (a) amplifying a portion of the MIF gene comprising a polymorphism associated with MIF expression; (b) contacting a solid substrate as described above for simultaneously genotyping a microsatellite repeat and a SNP with the amplified portion; and (c) determining whether the subject comprises a polymorphism associated with high MIF expression or whether the subject comprises a polymorphism associated with low MIF expression, thereby determining the MIF genotype of the subject.

In other aspects, the invention provides methods of treating diseases associated with high or low MIF expression.

In one aspect, the invention provides a method of treating anemia of chronic disease comprising administering to a subject a therapeutically effective amount of a MIF antagonist. In one embodiment, the subject is not responsive to erythropoietin (EPO) prior to the administration of the MIF antagonist. In one embodiment, the method further comprises administering to a subject a therapeutically effective amount of a MIF antagonist and one or more other agents that stimulate erythropoiesis. In one embodiment, the method further comprises administering EPO to the subject. In one embodiment, the method further comprises administering a TNFα antagonist or an IFNγ antagonist to the subject. The anemia of chronic disease may be caused by any condition, including a pathogenic infection, cancer, an autoimmune disease or disorder, a kidney disease or disorder, organ transplant rejection and aging. In one embodiment, the anemia of chronic disease results from malaria infection.

In another aspect, the invention provides a method of treating malaria comprising administering to a subject in need thereof a therapeutically effective amount of a MIF antagonist.

In another aspect, the invention provides a method of treating an infection comprising administering to a subject in need thereof a therapeutically effective amount of a MIF agonist. In one embodiment, the infection is a bacterial infection. In another embodiment, the infection is a viral infection or a retroviral infection. In another embodiment, the infection is a fungal infection. In one embodiment, the infection has resulted, or may result, in a respiratory disease. In one embodiment, the subject has a respiratory disease resulting from an infection. In another embodiment, the subject has pneumonia. In one embodiment, the subject has CAP. In another embodiment, the subject has meningitis. In another embodiment, the subject has influenza. In another embodiment, the subject has sepsis. In another embodiment, the subject is infected with HIV. In another embodiment, the subject is infected with HIV-1. In another embodiment, the subject is infected with a virus or pathogen that uses the CCR5 receptor.

In another aspect, the invention provides a method of attenuating the expression of CCR5 mRNA or protein in a subject with a disease associated with low MIF expression comprising the use of a MIF agonist.

In another aspect, the invention provides a method of inhibiting the life cycle of a virus that uses the CCR5 receptor during infection comprising administering to a subject infected with the virus, or at risk of being infected with the virus, a MIF agonist. In one embodiment, the virus is HIV-1. In one embodiment, the method further comprises administering to the subject another anti-viral agent.

In another aspect, the invention provides a method of treating HIV infection in a subject comprising administering to the subject a therapeutically effective amount of a MIF agonist. In one embodiment, the HIV infection is at an acute stage. In one embodiment, the method further comprises administering to the subject another anti-viral agent.

In one aspect, the invention provides a method of modulating the biological function of MIF, comprising the use of an agent that interacts modulates the interaction of CD44 with CD74.

In one embodiment, the invention provides a method of attenuating the biological function of MIF, comprising the use of an agent that inhibits the interaction between CD44 and CD74. The agent may be any agent. In one embodiment, the agent is selected from the group consisting of: a fragment of CD44, an extracellular fragment of CD44, an agent that binds CD44, an antibody or fragment thereof that binds to CD44, a small molecule, a small molecule mimic of chondroitin sulfate, heparin and a macromolecular mimic of chondroitin sulphate.

In another embodiment, the invention provides a method of attenuating the biological function of MIF, comprising the use of an agent that inhibits the expression of CD44. The agent may be any agent. In one embodiment, the agent is an siRNA or antisense polynucleotide that targets CD44.

In one embodiment, the invention provides a method of increasing the biological function of MIF, comprising the use of an agent that increases the interaction between MIF, CD44 and CD74.

In one embodiment, the invention provides a method of increasing the biological function of MIF, comprising the use of an agent that increases the interaction between CD44 and CD74.

The invention also provides novel methods of identifying potential agonists or antagonists of MIF. In one embodiment, the invention provides a method of identifying potential agonists or antagonists of MIF, comprising: (a) contacting a CD44 polypeptide, or a portion thereof, with a CD74 polypeptide, or portion thereof, in the presence and absence of a candidate agent; and (b) comparing the interaction of the CD44 and CD74 polypeptides in the presence of said candidate agent with the interaction in the absence of said candidate agent, wherein a candidate agent that enhances the interaction of the CD44 polypeptide and the CD74 polypeptide is identified as a potential agonist of MIF, and a candidate agent that inhibits the interaction of the CD44 polypeptide and the CD74 polypeptide is identified as a potential antagonist of MIF.

In another embodiment, the invention provides a method of identifying potential agonists or antagonists of MIF, comprising: (a) contacting a CD44 polypeptide or a portion thereof, with a MIF polypeptide or a portion thereof and a CD74 polypeptide or a portion thereof, in the presence and absence of a candidate agent; and (b) comparing the interaction of the CD44 polypeptide and the MIF and CD74 polypeptides in the presence of said candidate agent with the interaction in the absence of said candidate agent, wherein a candidate agent that enhances the interaction of the CD44 polypeptide and the MIF and CD74 polypeptides is identified as a potential agonist of MIF, and a candidate agent that inhibits the interaction of CD44 polypeptide and the MIF and CD74 polypeptides is identified as a potential antagonist of MIF.

In another aspect, the invention comprises a kit comprising: (a) at least one container means comprising one or more reagents for genotyping a subject for the presence of a polymorphism associated with high or low MIF expression, wherein said genotyping reagent is a polynucleotide probe, a polynucleotide primer, or a solid substrate that is capable of detecting a polymorphism associated with high or low MIF expression; and, (b) a label or instructions for use of the kit. In another embodiment, the invention comprises a kit comprising: (a) at least one container means comprising a premeasured dose of one or more MIF antagonists or MIF agonists; and, (b) a label or instructions for use of the kit.

### BRIEF DESCRIPTION OF THE DRAWINGS

FIG. 1 is a pair of graphs showing the dose-dependent impact of MIF, TNFα, and IFNγ on colony formation in murine bone marrow progenitor cultures in vitro. Bone marrow cells were harvested, plated in a methylcellulose-based medium, and colony numbers scored after the addition of murine cytokines. Individual assays were performed in duplicate, and the data shown is a compilation of 3-6 independently performed experiments. % inhibition of colony formation is calculated with reference to a cytokine-minus control. All values shown are the mean±SD and are significant when compared to wells with no cytokine addition (P<0.05). CFU-E: colony forming unit—erythroid; BFU-E: burst-forming unit—erythroid.

FIGS. 2A and 2B are graphs showing MIF inhibition of cytodifferentiation and hemoglobin production in human (K562) erythroid progenitors. FIG. 2A: terminal erythropoietic differentiation was assayed with diaminofluorene (DAF) after culture in differentiation medium together with MIF (200 ng/ml) for 96 hrs. The neutralizing anti-MIF mAb was added at 100 μg/ml. DAF positive cells were enumerated and expressed as fold-change over total, input cells. FIG. 2B: cellular hemoglobin content of cultured K562 progenitor cells. An isotypic control IgG₁ added in the same concentration had no impact on MIF's inhibitory action, nor did anti-MIF influence differentiation in the absence of MIF (not shown). Each value represents the mean±SD of at least three different experiments. *P<0.01 versus corresponding controls.

FIGS. 3A and 3B are graphs showing that malaria-infected, MIF-KO mice (MIF^{-/-}) suffer from less severe anemia and show increased survival when compared to genetically-matched, wild-type controls (MIF^{+/+}). FIG. 3A: time course for the development of anemia, as assessed by q.o.d. peripheral blood sampling. The data shown are the means±SD of 10 mice per group from one of two experiments, which yielded similar results. For differences in mean hemoglobin concentrations between the MIF^{+/+} and MIF^{-/-} mice, *P<0.01 for days 6, 8, 15, and *P<0.05 for days 10 and 12. Due to low numbers of survivors, the wild-type mice were not further studied after day 15. FIG. 3B: Kaplan-Meyer survival curves for MIF^{+/+} and MIF^{-/-} mice following infection with P. chabaudi AS. The data shown are for all mice studied (MIF^{+/+}: n=30; and MIF^{-/-}: n=31). The median survival was 13 days in the MIF^{+/+} mice and 15 days for the MIF^{-/-} mice, P=0.0113 (Mann-Whitney, two-tailed), and for overall survival, P<0.04 (χ2).

FIGS. 4A-4C show detection of a target nucleic acid by a biosensor chip. FIG. 4A is a schematic representation for the detection of the MIF CATT tetranucleotide repeat by ligation of biotinylated detection probe P2 to a set of capture probes P1 with different copies of CATT repeat immobilized on thin-film biosensor chip surface in the presence of certain CATT target (i.e. CATT 6). FIG. 4B shows an array template for the detection of the 5-, 6-, 7- and 8-CATT repeats, and the −173 G/C SNP. Oligonucleotides are arrayed in duplicates, as shown. +: positive control, an aldehyde modified dA20-biotin probe. FIG. 4C shows the visual appearance of the biosensor chip of the representative MIF genotypes.

FIG. 5 is a graph showing Kaplan-Meier survival curves for MIF −173 genotypes. The CC and CG genotypes were associated with improved survival in subjects with community-acquired pneumonia.

FIG. 6 is a bar graph showing the production of MIF by HIV-infected macrophages. Triplicate cultures of monocyte-derived macrophages were infected with HIV-1_{ADA} and cultivated until infection reached its peak (day 12, RT activity 10,500 cpm/μl). Cells were washed, and cultured in fresh medium. Aliquots of culture medium were withdrawn on the indicated days and analyzed by MIF-specific ELISA. Results are mean±SD. Statistical analysis using Student's t-test demonstrated significant differences between the amounts of MIF produced by mock-infected and HIV-infected cultures, p<0.05.

FIGS. 7A-7C are graphs showing that MIF suppresses HIV-1 replication in macrophages. FIG. 7A shows that anti-MIF MAb enhances HIV-1 replication in MDM cultures. Triplicate cultures of monocyte-derived macrophages were infected with HIV-1_{ADA} in the presence of anti-MIF MAb (25 μg/ml) or isotype control. Virus replication was monitored by RT activity in culture supernatants. Results are presented as mean±SD. FIG. 7B shows that recombinant MIF suppresses HIV-1 replication in MDM cultures. Macrophages were infected as in A and cultured without adding any reagent (control) or in the presence of recombinant MIF (50 ng/ml), polymyxin B (PMB, 10 μg/ml), or both agents together. Virus replication was monitored as in A. FIG. 7C shows that MIF-mediated inhibition of HIV-1 replication is reduced by anti-CD74 MAb. Macrophages were infected as in A and cultured in the presence of MIF (50 ng/ml) mixed with PMB (10 μg/ml) and anti-CD74 MAb (25 μg/ml). Control cultures were cultivated with PMB mixed with an isotype immunoglobulin with or without MIF.

FIG. 8 is a pair of graphs that show that MIF inhibits replication in PBMC of R5, but not X4, HIV-1 strain. Triplicate PHA-activated PBMC cultures were infected with R5 (ADA) or X4 (LAI) strains of HIV-1 and cultivated in the presence or absence (control) of recombinant MIF (50 ng/ml). At indicated time points after infection, reverse transcriptase activity was measured in culture supernatants. Results are mean±SD. Whereas differences between MIF-treated and control cultures of ADA-infected PBMCs are highly statistically significant (p<0.01), differences between MIF-treated and control cultures of LAI-infected cells are not significant (p>0.05).

FIG. 9 is a graph showing MIF downregulation of CCR5 expression in macrophages. Monocyte-derived macrophages were cultured in the presence of recombinant MIF (50 ng/ml) and polymyxin B (PMB, 10 μg/ml) for 48 h. Cell surface expression of CCR5 and CXCR4 was analyzed by FACS after staining with FITC-conjugated anti-CCR5 and PE-conjugated anti-CXCR4 antibodies (Pharmingen). Results (percent of receptor-positive cells) are shown as mean±SE for three performed experiments.

FIG. 10 is a schematic diagram of the structures of the human CD74 and CD44 proteins used to create stable cell lines. CD44^{Δ67} encodes a truncated CD44 lacking the cytoplasmic domain. IC, TM, and EC are the intracellular, transmembrane, and extracellular domains respectively. The location of the known intracytoplasmic serine phosphorylation sites are indicated.

FIGS. 11A-11D show that MIF-induced ERK-1/2 phosphorylation requires CD74 and full-length, intact CD44. FIG. 11A: COS-7/M6 cells stably-transfected with CD74, CD44, CD74+CD44, or CD74+CD44^{Δ67} were washed and stimulated with recombinant human MIF for the indicated times. Whole cell lysates then were prepared and analyzed by Western blotting with specific anti-phospho-ERK-1/2 (p-ERK1/2) or anti-total ERK/1/2 antibodies. FIG. 11B: primary murine embryonic fibroblasts (MEFs) or, FIG. 11C: peritoneal macrophages, were prepared from the genetically-defined mouse strains shown and stimulated with recombinant murine MIF. Peritoneal macrophages were stimulated for 10 mins (Mitchell et al. (1999). J Biol Chem 274:18100-6) and the lysates then analyzed by Western blotting. FIG. 11D: Pre-formed MIF/sCD74 complexes do not stimulate ERK-1/2 phosphorylation in CD44-expressing COS-7/M6 cells. Recombinant, sCD74 was incubated with MN overnight in a 3:1 molar ratio prior to addition to cells for 10 mins. Epidermal growth factor (EGF) was used as a positive control for ERK-1/2 phosphorylation (Yamamoto et al. (2003). Develop Biol 260:512-521). Data shown are representative of three experiments.

FIGS. 12A-12E show the phosphoserine content of CD74 and CD44 measured by ELISA. FIG. 12A: COS-7-derived cell lines and mouse embryonic fibroblasts (MEFs) were treated with MIF (100 ng/ml) for 10 mins and the cell lysates analyzed for phospho-serine by a CD74-specific sandwich ELISA. FIG. 12B: the COS-7/CD74+CD44 cell line was pre-treated with the protein kinase A (PKA) inhibitor, H-89 (20 μM), or the protein kinase C (PKC) inhibitor, RO-31-28801 (10 for 30 and 60 mins prior to MIF (100 ng/ml) stimulation. Cell lysates then were analyzed for phospho-serine content by capture ELISA. FIG. 12C: COS-7-derived cell lines and MEFs were treated with MIF (100 ng/ml) for 10 mins and the cell lysates analyzed for phospho-serine by a CD44-specific sandwich ELISA. FIG. 12D: analysis of CD44 phospho-serine content in control and MIF-stimulated, COS-7 cell lines after pre-incubation with the protein kinase A (PKA) inhibitor, H-89, or the protein kinase C (PKC) inhibitor, RO-31-2880, for 30 mins. The ELISA values represent the mean±SD of triplicate measurements. Experiments were replicated twice. *P<0.02 versus corresponding control (Student's T test, two-tailed). FIG. 12E: western analysis of COS-7/CD74+CD44 cells before and after stimulation with MIF (100 ng/ml for 10 minutes). Cell lysates were prepared and probed for PKA and PKC using phospho-specific, and total anti-PKA and anti-PKC antibodies.

FIGS. 13A and 13B are graphs showing the serum (A), and OVA-specific (B), immunoglobulin response in MIF^{+/+} and MIF^{-/-} mice. Results are the mean±SD from two independent experiments using 4-6 mice per group. *p<0.05, **p<0.01, ***p<0.001 for MIF^{+/+} versus MIF^{-/-} by Student's t-test (two-tailed).

FIG. 14 is a graph showing airway response curves in OVA-challenged and PBS-challenged mice. MIF^{+/+} and MIF^{-/-} mice were administered methacoline 12 hrs after the last challenge. Enhanced pause (Penh) values are expressed as mean±SEM, n=13 per group. *p<0.05 by Student's t-test (two-tailed). OVA-challenged mice (MIF^{+/+} and MIF^{-/-}) also showed a significant increase in Penh values when compared to PBS-challenged mice.

FIGS. 15A-15H are bar graphs showing leukocyte numbers in the BALF of MIF^{+/+} and MIF^{-/-} mice collected 16 hrs after challenge (A-E). Cell types were identified by morphological criteria. Eosinophil content additionally was quantitated by BALF eosinophil peroxidase (F). IL-5 and eoxtaxin were measured by ELISA (G,H). Results are the mean±SD for each group from two independent experiments using 4-6 mice per group. *p<0.005, **p<0.001, ***p<0.0005 for experimental conditions versus the OVA-challenged control by the Student's t-test (two-tailed).

FIG. 16 is a diagram of the human MIF gene showing transcriptional factor binding sites and the position of the of the −794 CATT tetranucleotide repeat (5-, 6-, 7-, and 8-CATT), and the −173 G/C SNP.

### DETAILED DESCRIPTION OF THE INVENTION

### 1. Overview

To provide an overall understanding of the invention, certain illustrative embodiments will now be described, including compositions and methods for: selecting a subject for treatment with a MIF agonist or antagonist, identifying a subject at risk for developing a disease associated with high or low MIF expression, predicting the severity of a disease associated with high or low MIF expression in a subject, and for predicting whether a subject is susceptible to a disease associated with high or low MIF expression. The invention also provides novel methods of diagnosing a patient for a disease associated with high or low MIF expression.

Also provided are novel methods for genotyping a subject for the presence of a polymorphism associated with high or low MIF expression comprising the use of a solid support or substrate (for example a chip or microarray) having polynucleotide probes attached thereto capable of simultaneously genotyping a microsatellite repeat and a SNP. This genotyping method may be used in a variety of contexts and to assess the status or genotype of a variety of individuals.

Methods for treating a subject having a disease or disorder associated with high or low MIF expression are also described.

Also provided are novel agonists of MIF that increase the interaction between CD74 and CD44, or that increase the interaction between MIF, CD74 and CD44. Also provided are novel antagonists of MIF that decrease (or inhibit) the interaction between CD74 and CD44, or that decrease (or inhibit) the interaction between MIF, CD74 and CD44.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

### 2. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The terms “comprise” and “comprising” are used in the inclusive, open sense, meaning that additional elements may be included.

The term “including” is used to mean “including but not limited to”. “Including” and “including but not limited to” are used interchangeably.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject. Agents can comprise, for example, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes. Agents may also comprise alcohols, alkyl halides, amines, amides, esters, aldehydes, ethers and other classes of organic agents.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a normatural arrangement.

The term “prophylactic” or “therapeutic” treatment is art-recognized and refers to administration of a drug to a patient. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

As used herein, the term “MIF” refers to macrophage migration inhibitory factor or active fragments thereof. Accession number EMBL Z23063 describes the nucleic acid sequence encoding human MIF (Bernhagen et al., Biochemistry 33:14144-14155 (1994)). An active fragment of MIF may comprise a fragment or a portion of the MIF protein encoding the tautomerase enzymatic activity of MIF, or a fragment that is capable of binding CD74.

As used herein a “MIF agonist” refers to any agent that mimics, activates, stimulates, potentiates or increases the biological activity of MIF. A MIF agonist may be MIF or a fragment thereof; an agent that mimics MIF (such as a small molecule); an agent that increases or enhances the expression of MIF, CD74 or CD44; an agent that enhances the binding of MIF to CD74; an agent than enhances the interaction between CD74 and CD44 (including, without limitation, a bivalent agent).

As used herein, the “biological function of MIF” refers to the ability of MIF to carry out one or more of the biological functions of MIF including, without limitation, sustaining immune cell survival or activation, promoting cytokine promotion, down-regulating CCR5, binding to CD74, activating MAP kinase signaling (e.g., ERK1/2, INK, and SAPK MAP kinase signaling), inhibiting p53, acting as a tautomerase, and/or acting as a thiol reductase.

As used herein a “MIF antagonist” refers to any agent that attenuates, inhibits, opposes, counteracts, or decreases the biological activity of MIF. A MIF antagonist may be an agent that inhibits or neutralizes MIF activity (including, without limitation, small molecules and anti-MIF antibodies); an agent that inhibits or decreases the expression of MIF (including, without limitation, an antisense molecule); an agent that inhibits or decreases the expression of the CD44 receptor (including, without limitation, an antisense molecule or an RNAi molecule); an agent that prevents the binding of MIF to CD74 (including, without limitation, an anti-CD74 antibody or an anti-MIF antibody or a fragment thereof); an agent that prevents the interaction between CD74 and CD44 (such as an anti-CD74 antibody or an anti-CD44 antibody or a fragment thereof); or an agent that prevents the interaction between CD74 and CD44. Examples of such molecules are fragments of CD74 and CD44, such as soluble fragments of such receptors. Examples of MIF antagonists have been disclosed previously, see, e.g., U.S. Pat. No. 6,774,227, Bernhagen et al., Nature 365, 756-759 (1993), Senter et al., Proc Natl Acad Sci USA 99:144-149 (2002); Dios et al., J. Med. Chem. 45:2410-2416 (2002); Lubetsky et al., J Biol Chem 277:24976-24982 (2002), which are hereby incorporated by reference.

As used herein, the term “treating” refers to preventing, slowing, delaying, stopping or reversing the progression of a condition.

As used herein, a “disease associated with high MIF expression” or a “disease associated with low MIF expression” is a disease associated with high or low MIF expression, respectively. This association can be established using well known methods. For example, diseases that are associated with high MIF expression include: autoimmunity, cancer, anemia of chronic disease, malaria, and asthma. Diseases that are associated with low, or insufficient, MIF expression include: infections (including viral, bacterial and fungal infections) and diseases resulting from, or caused by, infections, including respiratory diseases resulting from any infection, meningitis, pneumonia, CAP, influenza, sepsis, HIV infection, and infection with a pathogen that uses CCR5 as a receptor (such as HIV-1, Hepatitis C Virus (HCV), Epstein-Barr Virus, or Yersinia pestis).

As used herein, “anemia of chronic disease” refers to anemia that is immune driven. Anemia of chronic disease also known as “anemia of inflammation.” This condition can result from a condition selected from the group consisting of: a pathogenic infection, cancer, an autoimmune disease or disorder, a kidney disease or disorder, organ transplant rejection, and aging. See, e.g., Weiss and Goodnought, “Anemia of Chronic Disease”, N. Engl. J. Med. 352(10): 1011-23 (2005).

As used herein, “a polymorphism associated with MIF expression” refers to any polymorphisms in the MIF gene that correlate with high or low expression of the MIF gene, including without limitation: a single nucleotide polymorphism (G/C) at position −173 of the MIF promoter or the presence of five, six, seven or eight CATT repeats at position in the −794 region of the MIF promoter.

As used herein, “a polymorphism associated with low MIF expression” refers to the presence of a guanine (G) at position −173 of the MIF promoter or the presence five CATT boxes at position in the −794 region of the MIF promoter.

As used herein, “a polymorphism associated with high MIF expression” refers to the presence of a cytosine (C) at position −173 of the MIF promoter or the presence of six or more CATT boxes at position in the −794 region of the MIF promoter. (The positions of the MIF promoter are defined by reference to the nucleic acid sequence disclosed in EMBL Z23063.)

Each subject has two alleles corresponding to the MIF gene. As used herein, “a subject having a polymorphism associated with high MIF expression” refers to a subject that has a polymorphism associated with high MIF expression in at least one of its alleles. As used herein, “a subject having a polymorphism associated with low MIF expression” refers to a subject that has a G at position −173 of the MIF promoter in both alleles and that has five CATT repeats in the −794 region of the MIF promoter in both alleles. See, e.g., Table 1.

TABLE 1
Relationship between MIF genotype and MIF expression
levels in a subject:

| | |
|---|---|
| Genotype | Level |

| | | |
|---|---|---|
| CATT Box −794 Region | Position −173 | of MIF |

| Allele 1/Allele 2 | Allele 1/Allele 2 | High | Low |
|---|---|---|---|
| 5 CATT/5 CATT | G/G | | x |
| 5 CATT/5 CATT | G/C | x | |
| 5 CATT/5 CATT | C/G | x | |
| 5 CATT/5 CATT | C/C | x | |
| 5 CATT/X CATT | G/G | x | |
| 5 CATT/X CATT | G/C | x | |
| 5 CATT/X CATT | C/G | x | |
| 5 CATT/X CATT | C/C | x | |
| X CATT/X CATT | G/G | x | |
| X CATT/X CATT | G/C | x | |
| X CATT/X CATT | C/G | x | |
| X CATT/X CATT | C/C | x | |

X = at least 6 CATT Repeats

As used herein “higher risk” or “increased risk” refers to a statistically higher frequency of occurrence of the disease or condition. As used herein “lower risk” or “decreased risk” refers to a statistically lower frequency of occurrence of the disease or condition.

As used herein, the term “severity” of a disease, refers to the seriousness, degree or state of a disease or condition. For example, a disease may be characterized as mild, moderate or several. A person of skill in the art would be able to determine or assess the severity of a particular disease. For example, the severity of a disease may be determined by comparing the likelihood or length of survival of a subject having a disease with the likelihood or length of survival in other subjects having the same disease. In another embodiment, the severity of a disease may be determining by comparing the symptoms of the disease in a subject having a disease with the severity of the symptoms in other subjects having the same disease.

As used herein, the term “therapeutically effective amount” refers to the amount of a MIF agonist or antagonist (isolated or recombinantly produced), or a composition comprising a MIF agonist or antagonist, that is in sufficient quantities to treat a subject having, or at risk of developing, a disease associated with low or high MIF expression, or to treat a disease associated with high or low MIF expression itself. For example, an effective amount is sufficient to delay, slow, or prevent the onset or progression of a disease associated with high or low MIF expression, or related symptoms.

### 3. Prognostic and Diagnostic Methods

The invention comprises: (i) methods of diagnosing a patient for a disease associated with high or low MIF expression, (ii) methods of identifying patients at risk of developing a disease associated with high or low MIF expression, (iii) methods of predicting the severity of a disease associated with high or low MIF expression, (iv) methods of predicting the susceptibility of a patient to a disease associated with high or low MIF expression, (v) methods for selecting a patient for treatment with a MIF agonist or antagonist, and the like; comprising genotyping the subject for the presence of a polymorphism associated with high or low MIF expression.

A polymorphism associated with MIF expression may be any genetic alteration that modifies or correlates with the expression or activity of MIF. In one embodiment, the polymorphism associated with MIF expression is selected from the group consisting of: (i) the presence of five, six, seven or eight CATT repeats in the −794 region of the MIF promoter; and (ii) the presence of guanine or cytosine at position −173 of the MIF promoter. Polymorphisms associated with high MIF expression include, without limitation, the presence of six, seven or eight CATT repeats in the −794 region of the MIF promoter and the presence of a cytosine (C) at position −173 of the MIF promoter. Polymorphisms associated with low MIF expression include, without limitation, the presence of five CATT repeats in the −794 region of the MIF promoter and the presence of a guanine (G) at position −173 of the MIF promoter. In general, the greater number of CATT repeats that are present in the −794 region of the MIF promoter, the greater the expression and/or activity of MIF. The above polymorphisms are illustrative of polymorphisms that may be associated with MIF expression. Nevertheless, the present invention encompasses all other polymorphisms that are associated with the expression or activity of the MIF gene. As an illustrative embodiment, polymorphisms consisting of a G/A or G/T nucleotide change at position −173 of the MIF promoter may be associated with high or low MIF expression. In another illustrative embodiment, polymorphisms the presence of two, three, four, nine, ten, eleven or twelve or more CATT repeats in the −794 region of the MIF promoter may be associated with high or low MIF expression. Methods of genotyping a subject for the presence of a polymorphism (including single nucleotide polymorphisms and microsatellite repeats) in a gene are well known and routinely used in the art. Exemplary methods of genotyping a subject for the presence of a polymorphism in the MIF gene are described below.

Each subject has two alleles corresponding to the MIF gene. A subject having a polymorphism associated with low MIF expression refers to a subject having a G at position −173 of the MIF promoter in both alleles of the MIF gene and having five CATT boxes in the −794 region of the MIF promoter in both alleles of the MIF gene. See, e.g., Table I.

A subject having a polymorphism associated with high MIF expression refers to a subject having a polymorphism associated with high MIF expression in at least one of its alleles. (See Table I.) In a preferred embodiment, a subject having a polymorphism associated with high MIF expression refers to (i) a subject that has more than 6 CATT boxes in both alleles of the MIF gene, or (ii) a subject having a C at position −173 of the MIF gene in each of the two alleles of the MIF gene and having 6 or more CATT boxes in at least one of the two alleles of the MIF gene. In another preferred embodiment, a subject having a polymorphism associated with high MIF expression refers to a subject having 7 CATT repeats in at least one of the two alleles in the −794 region of the MIF promoter and having a C at position −173 in at least one of the two alleles of the MIF gene.

The methods described herein can be used in connection with any disease that is associated with high or low MIF expression.

Diseases associated with high MIF expression include, without limitation, diseases caused by infection by a protozoan, such as malaria; anemia of chronic disease; and asthma.

Diseases associated with low MIF expression include, without limitation: infections (in particular acute infections) and the diseases caused by infections. In one embodiment, the disease associated with low MIF expression is a respiratory disease caused by an infection, including without limitation, infection by gram positive and gram negative bacteria (e.g., Legionella), mycobacteria (such as Mycobacterium tuberculosis or other Mycobacterium species), fungal infections (e.g., infections of Pneumocystis, Candida, and Histoplasma) and viral infections (e.g., infections of influenza, varicella, and corona virus such as SARS-associated coronoavirus). In another embodiment, the disease associated with low MIF expression is sepsis. In another embodiment, the disease associated with low MIF expression is an infection is pneumonia (regardless of whether it is caused by a bacterial, viral or fungal infection). In a specific embodiment, the pneumonia is Community Acquired Pneumonia (CAP). In another embodiment, the disease associated with low MIF expression is meningitis. In another embodiment, the disease associated with low MIF expression is influenza. Microbial infections that lead to pneumonia include bacterial infections (e.g., infections of gram positive bacteria, gram negative bacteria, and mycobacteria such as mycobacterium tuberculosis), fungal infections (e.g., infections of Pneumocystis, Candida, and Histoplasma) and viral infections (e.g., infections of influenza, varicella, and corona virus such as SARS-associated coronoavirus). In another embodiment, a disease associated with low MIF expression is infection by a virus or other pathogen that use the CCR5 receptor for infection, for example Human Immunodeficiency Virus-1 (HIV-1), Hepatitis C Virus (HCV), Epstein-Barr Virus, or Yersinia Pestis.

In one embodiment, the methods of the invention are useful for selecting a subject for treatment with a MIF antagonist, wherein the subject has a disease or is at risk of developing a disease associated with high MIF expression. Such methods comprise genotyping the subject for the presence of a polymorphism associated with MIF expression. A subject having a polymorphism associated with high MIF expression is selected for treatment with a MIF antagonist. MIF antagonists are useful for treating a subject having, or is at risk of developing, a disease associated with high MIF expression. In one embodiment, the subject has or is at risk of developing a disease caused by infection by a protozoan. In one embodiment, the subject has or is at risk of developing malaria. In another embodiment, the subject has or is at risk of developing anemia of chronic disease. In another embodiment, the subject has or is at risk of developing asthma.

In another embodiment, the methods of the invention are useful for selecting a subject for treatment with a MIF agonist, wherein the subject has a disease or is at risk of developing a disease associated with low MIF expression. Such methods comprise genotyping the subject for the presence of a polymorphism associated with MIF expression, wherein a subject having a polymorphism associated with low MIF expression is selected for treatment with a MIF agonist. MIF agonists are useful for treating a subject having, or is at risk of developing, a disease associated with low MIF expression. In one embodiment, the subject has, or is at risk of being infected with, a pathogen and/or of developing a disease caused by an infection with a pathogen. In one embodiment, the subject has, or is at risk of developing, sepsis. In another embodiment, the subject has, or is at risk of developing, an infection that leads to a respiratory disease or has a respiratory disease caused by an infection. In another embodiment, the subject has, or is at risk of developing, pneumonia. In another embodiment, the subject has, or is at risk of developing, CAP. In another embodiment, the subject has, or is at risk of developing, meningitis. In another embodiment, the subject has, or is at risk of developing, influenza. In another embodiment, the subject is infected with, or is at risk of being infected with, a pathogen that uses the CCR5 as a receptor, or has, or is at risk of developing, a disease caused by infection with a pathogen that uses the CCR5 receptor. In one embodiment, the subject is infected with, or is at risk of being infected with, HIV-1. In other embodiments, the subject is infected with, or is at risk of being infected with, Hepatitis C Virus (HCV), Epstein-Barr Virus, or Yersinia Pestis.

In another embodiment, the methods of the invention are useful for identifying a subject at risk for developing a disease associated with high MIF expression. Such methods comprise genotyping the subject for the presence of a polymorphism associated with high or low MIF expression. A subject having a polymorphism associated with high MIF expression is at a higher risk of developing a disease associated with high MIF expression than a subject having a polymorphism associated with low MIF expression. A subject having a polymorphism associated with low MIF expression is at a lower risk of developing a disease associated with high MIF expression than a subject having a polymorphism associated with high MIF expression. In one embodiment, the subject is at risk of developing a disease caused by infection by a protozoan. In another embodiment, the subject is at risk of developing malaria. In another embodiment, the subject is at risk of developing anemia of chronic disease.

In another embodiment, the methods of the invention are useful for identifying a subject at risk for developing a disease associated with low MIF expression. Such methods comprise genotyping the subject for the presence of a polymorphism associated with high or low MIF expression. A subject having a polymorphism associated with low MIF expression is at a higher risk of developing a disease associated with low MIF expression than a subject having a polymorphism associated with high MIF expression. A subject having a polymorphism associated with high expression is at a lower risk of developing a disease associated with low MIF expression than a subject having a polymorphism associated with low MIF expression. In one embodiment, the subject is at risk of developing a disease caused by an infection. In another embodiment, the subject is at risk of developing sepsis. In another embodiment, the subject is at risk of developing an infection that leads to a respiratory disease or is at risk of developing a respiratory disease caused by an infection. In another embodiment, the subject is at risk of developing pneumonia (for example CAP). In another embodiment, the subject is at risk of developing meningitis. In another embodiment, the subject is at risk of developing influenza. In another embodiment, the subject is at risk of developing a disease caused by an infection with a pathogen that uses the CCR5 receptor. In another embodiment, the subject at risk of developing an HIV infection or is at risk of developing AIDS. In other embodiments, the subject is at risk of developing a disease caused by an infection with HCV, Epstein-Barr Virus, or Yersinia pestis.

In other embodiments, the methods of the invention are useful for predicting the severity of a disease associated with high MIF expression in a subject. Such methods comprise genotyping the subject for the presence of a polymorphism associated with MIF expression. A subject having a polymorphism associated with high MIF expression is at a higher risk for, or has a greater likelihood of, developing a more severe disease associated with high MIF expression than a subject having a polymorphism associated with low MIF expression. A subject having a polymorphism associated with low MIF expression has a greater likelihood of developing a milder (i.e. less severe) form of a disease associated with high MIF expression than a subject having a polymorphism associated with high MIF expression. In one embodiment, the disease associated with high MIF expression is caused by infection with a protozoan. In another embodiment, the disease associated with high MIF expression is malaria. In another embodiment, the disease associated with high MIF expression is anemia of chronic disease. A person of skill in the art would be able to determine or assess the severity of a particular disease. For example, the severity of a disease may be determined by comparing the likelihood or length of survival of a subject having a disease with the likelihood or length of survival in other subjects having the same disease. In another embodiment, the severity of a disease may be determining by comparing the symptoms of the disease in a subject having a disease with the severity of the symptoms in other subjects having the same disease.

In one embodiment, the invention provides a method for predicting the severity of asthma in a patient having asthma, or at risk of developing asthma, comprising genotyping the subject for the presence of a polymorphism associated with MIF expression. A subject having a polymorphism associated with high MIF expression is at a higher risk for, or has a greater likelihood of, developing more severe asthma than a subject having a polymorphism associated with low MIF expression. A subject having a polymorphism associated with low MIF expression has a greater likelihood of developing a milder asthma than a subject having a polymorphism associated with high MIF expression. The severity of asthma can be determined by using any method. In one embodiment, the severity of asthma is determined by using the GINA criteria outlined by WHO (which classifies asthma as intermittent; mild persistent, moderately persistent or severe persistent), by measuring airflow obstruction in the lungs (for example, by measuring airflow obstruction by spirometry or peak expiratory flow (PEF) or by measuring the forced expiratory volume in one second (FEV₁)), or by determining the need for steroid and immunosuppressive medication (See Example 7).

In another embodiment, the methods of the invention are useful for predicting the severity of a disease associated with low MIF expression in a subject. Such methods comprise genotyping the subject for the presence of a polymorphism associated with MIF expression. A subject having a polymorphism associated with low MIF expression is at a higher risk for developing a more severe form of a disease associated with low MIF expression than a subject having a polymorphism associated with high MIF expression. A subject having a polymorphism associated with high MIF expression has a greater likelihood of developing a milder (i.e. less severe) form of a disease associated with low MIF expression. In one embodiment, the disease associated with low MIF expression is a disease caused by an infection, particularly a disease caused by an acute infection or a disease caused by a respiratory infection. In another embodiment, the disease associated with low MIF expression is sepsis. In another embodiment, the disease associated with low MIF expression is an infection leading to a respiratory disease. In another embodiment, the disease associated with low MIF expression is pneumonia. In another embodiment, the disease associated with low MIF expression is CAP. In one embodiment, the disease associated with low MIF expression is meningitis. In one embodiment, the disease associated with low MIF expression is influenza. In one embodiment, the disease associated with low MIF expression is infection by a pathogen that uses the CCR5 receptor, or a disease caused by infection with a pathogen that uses the CCR5 receptor. In another embodiment, the disease associated with low MIF expression is infection with HIV or AIDS. In other embodiments, the disease associated with low MIF expression is infection with HCV, Epstein-Barr Viruse, or Yersinia pestis.

In another embodiment, the methods of the invention are useful for predicting whether a subject is susceptible to a disease that is associated with high MIF expression. Such methods comprise genotyping a subject for the presence of a polymorphism associated with high or low MIF expression. A subject having a polymorphism associated with high MIF expression is more susceptible to a disease associated with high MIF expression than a subject having a polymorphism associated with low MIF expression. A subject having a polymorphism associated with low MIF expression is less susceptible to a disease associated with high MIF expression than a subject having a polymorphism associated with high MIF expression. In one embodiment, the disease associated with high MIF expression is caused by infection of a protozoan. In another embodiment, the disease associated with high MIF expression is malaria. In another embodiment, the disease associated with high MIF expression is anemia of chronic disease.

In another embodiment, the methods of the invention are useful for predicting whether a subject is susceptible to a disease that is associated with low MIF expression. Such methods comprise genotyping a subject for the presence of a polymorphism associated with high or low MIF expression. A subject having a polymorphism associated with low MIF expression is more susceptible to a disease associated with low MIF expression than a subject having a polymorphism associated with high MIF expression. A subject having a polymorphism associated with high MIF expression is less susceptible to a disease associated with low MIF expression than a subject having a polymorphism associated with low MIF expression. In one embodiment, the disease associated with low MIF expression is a disease caused by an infection, particularly a disease caused by an acute infection. In one embodiment, the disease associated with low MIF expression is sepsis. In one embodiment, the disease associated with low MIF expression is an infection leading to a respiratory disease or a respiratory disease caused by an infection. In one embodiment, the disease associated with low MIF expression is pneumonia. In one embodiment, the disease associated with low MIF expression is CAP. In one embodiment, the disease associated with low MIF expression is meningitis. In one embodiment, the disease associated with low MIF expression is influenza. In one embodiment, the disease associated with low MIF expression is infection by a pathogen that uses the CCR5 receptor, or a disease caused by infection with a pathogen that uses the CCR5 receptor. In another embodiment, the disease associated with low MIF expression is infection with HIV or AIDS. In other embodiments, the disease associated with low MIF expression is infection with HCV, Epstein-Barr Viruse, or Yersinia pestis.

Genotyping Assays

Certain aspects of the invention comprise the step of genotyping a subject for the presence of a polymorphism associated with MIF expression (e.g., high or low MIF expression). Any assay that permits detection of a polymorphism in the MIF gene (which is used herein to include the MIF coding region and the MIF promoter region) may be used in the claimed methods. The preferred method for detecting a polymorphism will depend, in part, upon the molecular nature of the polymorphism. For example, certain methods may be amenable to the detection of insertions, deletions, substitutions, repeats, or single nucleotide polymorphisms (SNPs). Such assays are well known in the art and may encompass, for example, DNA sequencing, hybridization, ligation, or primer extension methods.

In one embodiment, the step of genotyping a subject may comprise contacting a sample obtained from the subject with a polynucleotide probe that hybridizes specifically to a polymorphism associated with MIF expression, and, determining whether hybridization occurs. The polynucleotide probe can be engineered to hybridize specifically to a polymorphism associated with high MIF expression, but not to a polymorphism associated with low MIF expression. Alternatively, the polynucleotide probe can be engineered to hybridize specifically to a polymorphism associated with low MIF expression, but not to a polymorphism associated with high MIF expression. Hybridization of the probe to the DNA in the sample indicates whether the subject comprises a polymorphism associated with high MIF expression or a polymorphism associated with low MIF expression, thereby genotyping the subject for the presence of a polymorphism associated with MIF expression. In certain embodiments, methods for genotyping a subject for the presence of a polymorphism that is associated with MIF expression further comprises contacting a sample obtained from the subject with a control polynucleotide probe. A control polynucleotide probe will not, for example, hybridize specifically to a polymorphism associated with high or low MIF expression. The polynucleotide probe may comprise nucleotides that are fluorescently, radioactively, or chemically labeled to facilitate detection of hybridization.

Hybridization may be performed and detected by standard methods known in the art, such as by Northern blotting, Southern blotting, fluorescent in situ hybridization (FISH), or by hybridization to polynucleotides immobilized on a solid support, such as a DNA array or microarray. Array elements may comprise any polynucleotide, including genomic DNA, cDNA, synthetic DNA or other types of nucleic acid array elements.

In one embodiment, the probe is a DNA probe that is immobilized on a solid support, such as a DNA array or microarray. In one embodiment, the probe is from about 8 nucleotides to about 500 nucleotides.

In another embodiment, a subject is genotyped for the presence of a polymorphism associated with MIF expression by hybridization to a DNA array or microarray, by incorporation of biotinylated primers followed by avidin-enzyme conjugate detection, or by incorporation of ³²P-labeled deoxynucleotide triphosphates, such as dCTP or dATP, into the target polynucleotides (e.g., a polynucleotide that may include a polymorphism that is associated with MIF expression). Hybridization may be detected, for example, by measuring the intensity of the labeled probe remaining on a DNA array after washing.

Methods of detecting a polymorphism associated with MIF expression may include amplification of a region of DNA that comprises a polymorphism that is associated with MIF expression. Any method of amplification may be used. In one specific embodiment, a region of DNA comprising the variation is amplified by using polymerase chain reaction (PCR). PCR was initially described by Mullis (See e.g., U.S. Pat. Nos. 4,683,195 4,683,202, and 4,965,188, herein incorporated by reference), which describes a method for increasing the concentration of a region of DNA, in a mixture of genomic DNA, without cloning or purification. Other PCR methods may also be used for nucleic acid amplification, including but not limited to RT-PCR, quantitative PCR, real time PCR, Rapid Amplified Polymorphic DNA Analysis, Rapid Amplification of cDNA Ends (RACE), rolling circle amplification, or multiple displacement amplification. For example, polynucleotide primers that flank the MIF gene (including the MIF promoter) are combined with a DNA mixture. The mixture also includes the necessary amplification reagents (e.g., deoxyribonucleotide triphosphates, buffer, etc.) necessary for the thermal cycling reaction. According to standard PCR methods, the mixture undergoes a series of denaturation, primer annealing, and polymerase extension steps to amplify the region of DNA that comprises a polymorphism that is associated with MIF expression. The length of the amplified region of DNA is determined by the relative positions of the primers with respect to each other, and therefore, this length is a controllable parameter. For example, hybridization of the primers may occur such that the ends of the primers proximal to the variation are separated by 1 to 10,000 base pairs (e.g., 10 base pairs (bp) 50 bp, 200 bp, 500 bp, 1,000 bp, 2,500 bp, 5,000 bp, or 10,000 bp).

In other embodiments, methods for genotyping a subject for the presence of a polymorphism that is associated with MIF expression comprise: (a) contacting a sample obtained from the subject with a pair of amplification primers, wherein said primers are capable of amplifying a portion of the MIF promoter comprising a polymorphism associated with MIF expression; (b) amplifying DNA in the sample, thereby producing amplified DNA; and (c) determining whether the amplified DNA comprises a polymorphism associated with high MIF expression or a polymorphism associated with low MIF expression, thereby genotyping the subject for the presence of a polymorphism associated with MIF expression. The step of determining whether the amplified DNA comprises a polymorphism associated with high or low MIF expression can be carried out using any method known in the art and/or described herein. The method may further comprise sequencing the amplified DNA.

In one embodiment, the presence of a polymorphism associated with MIF expression is detected and/or determined by DNA sequencing. Any of a variety of sequencing reactions known in the art can be used to directly sequence the allele. Examples of sequencing reactions include those based on techniques developed by Maxim and Gilbert ((1977) Proc. Natl. Acad. Sci. USA 74:560) or Sanger (Sanger et al (1977) Proc. Nat. Acad. Sci. USA 74:5463). DNA sequence determination may be performed by standard methods such as dideoxy chain termination technology and gel-electrophoresis, or by other methods such as by pyrosequencing (Biotage AB, Uppsala, Sweden). For example, DNA sequencing by dideoxy chain termination may be performed using unlabeled primers and labeled (e.g., fluorescent or radioactive) terminators. Alternatively, sequencing may be performed using labeled primers and unlabeled terminators. It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv. Chromatogr. 36:127-162; and Griffin et al. (1993) Appl. Biochem. Biotechnol. 38:147-159). The nucleic acid sequence of the DNA in the sample can be studied to determine whether a polymorphism associated with high or low MIF expression is present. It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In another embodiment, the presence of a polymorphism associated with MIF expression is detected and/or determined using FISH. For example, a probe that specifically hybridizes to a polymorphism associated with MIF expression is hybridized to a subject's genomic DNA by FISH. FISH can be used, for example, in metaphase cells, to detect a deletion or repeat region in genomic DNA. Genomic DNA is denatured to separate the complimentary strands within the DNA double helix structure. The polynucleotide probe of the invention is then added to the denatured genomic DNA. In a specific embodiment, a probe that specifically hybridizes to a polymorphism associated with high MIF expression is used. Accordingly, if a polymorphism associated with high MIF expression is present, the probe will hybridize to the genomic DNA. The probe signal (e.g., fluorescence) can then be detected through a fluorescent microscope for the presence of absence of signal. The presence of signal, therefore, indicates the presence of a polymorphism associated with high MIF expression. In another specific embodiment, a probe that specifically hybridizes to a polymorphism associated with low MIF expression is used. In this case, if a polymorphism associated with low MIF expression is present, the probe will hybridize to the genomic DNA. The probe signal (e.g., fluorescence) can then be detected through a fluorescent microscope for the presence of absence of signal. The presence of signal, therefore, indicates the presence of a polymorphism associated with low MIF expression.

In another embodiment, the presence of a polymorphism associated with MIF expression is detected and/or determined by primer extension with DNA polymerase. In one embodiment, a polynucleotide primer of the invention hybridizes immediately adjacent to the polymorphism. A single base sequencing reaction using labeled dideoxynucleotide terminators may be used to detect the polymorphism. In one embodiment, the presence of a polymorphism associated with high or low MIF expression will result in the incorporation of the labeled terminator, whereas the absence of a polymorphism associated with high or low MIF expression will not result in the incorporation of the terminator. In another embodiment, the dideoxynucleotides may be labeled (e.g., fluorescently, radioactively, chemically, etc.) and the polymorphism is detected by detecting the incorporation of the labeled dideoxynucleotides during or after primer extension. In another embodiment, a polynucleotide primer of the invention hybridizes specifically to a polymorphism associated with high or low MIF expression. The presence of a polymorphism will result in primer extension, whereas the absence of a polymorphism will not result in primer extension. The primers and/or nucleotides may further include fluorescent, radioactive, or chemical probes. A primer labeled by primer extension may be detected by measuring the intensity of the extension product, such as by gel electrophoresis, mass spectrometry, or any other method for detecting fluorescent, radioactive, or chemical labels.

In another embodiment, the presence of a polymorphism associated with MIF expression is detected and/or determined by ligation. In one embodiment, a polynucleotide primer hybridizes specifically to a polymorphism associated with high or low MIF expression. A second polynucleotide that hybridizes to a region of the MIF gene immediately adjacent to the first primer is also provided. One, or both, of the polynucleotide primers may be fluorescently, radioactively, or chemically labeled. Ligation of the two polynucleotide primers will occur in the presence of DNA ligase if a polymorphism associated with high or low MIF expression is present. Ligation may be detected by gel electrophoresis, mass spectrometry, or by measuring the intensity of fluorescent, radioactive, or chemical labels.

For example, identification of a polymorphism can be carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to identify a polymorphism. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res. 24: 3728), OLA combined with PCR permits typing of two alleles in a single microliter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of polymorphisms using a high throughput format that leads to the production of two different colors.

In another embodiment, the presence of a polymorphism associated with MIF expression is detected and/or determined by single-base extension (SBE). For example, a fluorescently-labeled primer that is coupled with fluorescence resonance energy transfer (FRET) between the label of the added base and the label of the primer may be used. Typically, the method, such as that described by Chen et al., (PNAS 94:10756-61 (1997), incorporated herein by reference) uses a locus-specific polynucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

In certain embodiments, a polymorphism that is associated with MIF expression may be detected using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately adjacent 3′ to the polymorphic site is permitted to hybridize to a target molecule obtained from a subject. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For SNPs that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

In a further embodiment, protection from cleavage agents (such as a nuclease, hydroxylamine or osmium tetraoxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the one of the polymorphic alleles with the sample. The double-stranded duplexes are treated with an agent that cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662).

Commercial assays, such as the Taqman assay (Applied Biosystems, Foster City, Calif.), may also be used for genotyping a subject for the presence of a polymorphism that is associated with MIF expression. Genotyping and uses therefore are shown in U.S. Ser. Nos. 10/442,021, 10/013,598 (U.S. Patent Application Publication 20030036069), and U.S. Pat. Nos. 5,925,525, 6,268,141, 5,856,092, 6,267,152, 6,300,063, 6,525,185, 6,632,611, 5,858,659, 6,284,460, 6,361,947, 6,368,799, 6,673,579 and 6,333,179.

Polynucleotides used in any of the methods of the invention, including probes and primers, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotide probes of the invention may hybridize to a segment of target DNA such that the variation aligns with a central position of the probe, or the variation may align with a terminal position of the probe.

Standard instrumentation known to those skilled in the art can be used for the amplification and detection of amplified DNA. For example, a wide variety of instrumentation has been developed for carrying out nucleic acid amplifications, particularly PCR, e.g. U.S. Pat. No. 5,038,852 (computer-controlled thermal cycler); Wittwer et al., Nucleic Acids Research, 17: 4353-4357 (1989) (capillary tube PCR); U.S. Pat. No. 5,187,084 (air-based temperature control); Garner et al, Biotechniques, 14: 112-115 (1993) (high-throughput PCR in 864-well plates); International application No. PCT/US93/04039 (PCR in micro-machined structures); European patent application No. 90301061.9 (publ. No. 0381501 A2) (disposable, single use PCR device), and the like. In certain embodiments, the invention described herein utilizes real-time PCR or other methods known in the art such as the Taqman assay.

MIF Polymorphism Detection Using Immobilized Probes

In certain embodiments, a polymorphism in the MIF gene that is associated with MIF expression may be detected using polynucleotide probes that have been immobilized on a solid support or substrate. Immobilized polynucleotide probes hybridize to a region of the MIF gene (including the promoter region of the MIF gene) that comprises a polymorphism that is associated with MIF expression. The present invention may employ any solid substrate known in the art, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes. Patents that describe synthesis techniques in specific embodiments include U.S. Pat. Nos. 5,412,087, 6,147,205, 6,262,216, 6,310,189, 5,889,165, and 5,959,098.

In a specific embodiment, the invention provides a solid support or substrate for simultaneously genotyping a microsatellite repeat and a SNP in the MIF gene (including the promoter region). Examples of solid supports and substrates include, without limitation, a nucleic acid probe array (e.g., a chip, a microarray, or an array), a nitrocellulose filter, a microwell, a bead, a sample tube, a microscope slide, a microfluidics device, and the like. The solid support may be made of various materials, including paper, cellulose, nylon, polystyrene, polycarbonate, plastics, glass, ceramic, stainless steel, or the like. The solid support may have a rigid or semi-rigid surface, and may be spherical (e.g., bead) or substantially planar (e.g., flat surface) with appropriate wells, raised regions, etched trenches, or the like. The solid support may also include a gel or matrix in which nucleic acids may be embedded or fibers or any solid support comprising bound nucleic acids. The solid support comprises at least two polynucleotide probes that are complementary to one or more polymorphisms associated with MIF expression. In a preferred embodiment, at least one of the probes detects a microsatellite repeat associated with MIF expression and at least one of the other probes detects a SNP associated with MIF expression. Hybridization to the polynucleotide probes can be detected using any of the detection method. In one embodiment, hybridization may be detected by the naked eye, without the aid of instruments for visualizing hybridization. Platforms for detection by the naked eye include thin-film technologies such as those described in Jenison et al., Expert Rev. Mol. Diagn. 6:89-99 (2006); Ostroff et al., Clinical Chemistry 45:1659-1664 (1999) and Zhong et al., PNAS 100:11559-11564 (2003), which are hereby incorporated by reference.

Thus, in a preferred embodiment, the invention provides the use of thin film technology to simultaneously genotype a microsatellite repeat and a SNP in the MIF gene. (See Example 2). For example, in one embodiment, the invention provides the use of a thin film chip or microarray. Thin-film technology permits the visual detection of nucleic acid targets with the unaided eye. The assay is inexpensive, robust, highly specific, rapid and easy to use, thus permitting its implementation in rural settings with limited technology. See Jenison et al. (2006) Expert Rev. Mol. Diagn. 6:89-99.

Thin film technology is capable of generating a visual signal by the direct interaction of light with thin films formed on a solid surface (e.g., a silicon surface). The surface is constructed to be antireflective to specific wavelengths of light by the addition of antireflective coatings that create destructive interference. When light reflected from the surface-thin-film interface is out of phase with light reflected from the air-thin-film interface, specific wavelengths of light are eliminated from the reflected light by destructive interference, creating a characteristic surface color. Optical thickness of the thin film, which is a function of both refractive index and physical thickness, determines which wavelengths of light are antireflected. Changes in the optical thickness of the thin film will result in a visible color change on the surface, once it is dried. This optical principle has been exploited to configure biologic assays on optical surfaces that transduce a thickness change into a surface color change that is a direct measure of interactions between target molecules in solution and capture molecules on the surface of the chip. The method is sensitive to thickness changes in the angstrom range, translating into highly sensitive detection of target molecules in very rapid assay formats.

By amplifying molecular interactions, the increased mass deposited on the surface of a thin film chip can be visually detected. Thin film formation can be accomplished by a variety of signal amplification techniques, such as by the enzymatic turnover of precipitating substrates. For the detection of nucleic acid sequences, thin film development may utilize, for example, the detection of biotin-labeled probes by binding of an antibiotin antibody conjugated to horseradish perixidase (HRP). In the presence of a precipitating substrate for HRP, an enhanced molecular thin film is deposited onto the surface of the solid substrate. Control of the reflective properties to create, for example, a gold-colored surface is achieved by the coating of surfaces with optical layers of defined refractive index and thickness, using well-established semiconductor processes. Details of the preparation of the optically coated surfaces have been described previously. See, for example, Jenison et al. (2001). Biotech. 19:62-65. Briefly, the base surface of the chip is crystalline silicon, which provides a highly reflective, inert and molecularly flat surface to which the antireflective coating (silicon nitride) is applied by vapor deposition (e.g., to a thickness of 475 angstroms). An attachment layer, such as T-structure aminoalkyl polydimethy siloxane (TSPS) can be coated on the surface to provide better immobilization of biological materials, such as nucleic acid capture probes or antibodies.

In one embodiment, a solid support as described above (such as a chip or microarray) comprises at least one probe that hybridizes specifically to a guanine or to a cytosine at position −173 of the MIF promoter.

In another embodiment, a solid support as described above (such as a chip or microarray) comprises at least one probe that hybridizes specifically to a sequence selected from the group consisting of SEQ ID NO: 1 (CATTCATTCATTCATTCATT), SEQ ID NO: 2 (CATTCATTCATTCATTCATTCATT), SEQ ID NO: 3 (CATTCATTCATTCATTCATTCATTCATT), and SEQ ID NO: 4 (CATTCATTCATTCATTCATT CATTCATTCATT).

In another embodiment, a solid support as described above (such as a chip or microarray) comprises: (a) at least one probe that hybridizes specifically to a guanine or a cytosine at position −173 of the MIF promoter; and, (b) at least one probe that hybridizes specifically to a sequence selected from the group consisting of SEQ ID NO: 1, SEQ ID NO: 2, SEQ ID NO: 3, and SEQ ID NO: 4.

In another embodiment, a solid support as described above (such as a chip or microarray) comprises a probe that hybridizes specifically to a guanine at position −173 of the MIF promoter and another probe that hybridizes specifically to a cytosine at position −173 of the MIF promoter.

In another embodiment, a solid support as described above (such as a chip or microarray) comprises: (a) a probe that hybridizes specifically to SEQ ID NO: 1; (b) a probe that hybridizes specifically to SEQ ID NO: 2; (c) a probe that hybridizes specifically to SEQ ID NO: 3; and, (d) a probe that hybridizes specifically to SEQ ID NO: 4.

In another embodiment, a solid support as described above (such as a chip or microarray) comprises: (a) a probe that hybridizes specifically to a guanine at position −173 of the MIF promoter; (b) a probe that hybridizes specifically to a cytosine at position −173 of the MIF promoter; (c) a probe that hybridizes specifically to SEQ ID NO: 1; (d) a probe that hybridizes specifically to SEQ ID NO: 2; (e) a probe that hybridizes specifically to SEQ ID NO: 3; and, (f) a probe that hybridizes specifically to SEQ ID NO: 4.

In one embodiment, the invention provides a method of determining the MIF genotype of a subject comprising contacting a solid substrate as disclosed herein with a sample obtained from the subject and determining the MIF genotype of the subject. The sample may be amplified prior to contacting the sample with the solid substrate disclosed herein. In one embodiment, the invention provides a method of determining the MIF genotype of a subject comprising: (a) amplifying a portion of the MIF gene comprising a polymorphism associated with MIF expression; (b) contacting a solid substrate as disclosed herein with the amplified portion; and, (c) determining whether the subject comprises a polymorphism associated with high MIF expression or whether the subject comprises a polymorphism associated with low MIF expression, thereby determining the MIF genotype of the subject.

Other Genotyping Methods

Moreover, the genotyping methods disclosed herein can be substituted by the use of other methods that can establish whether a subject expresses MIF at high or low levels. Such methods are therefore useful for diagnosing a patient for a disease associated with high or low MIF expression, identifying patients at risk of developing a disease associated with high or low MIF expression, predicting the severity of a disease associated with high or low MIF expression, predicting the susceptibility of a patient to a disease associated with high or low MIF expression, or selecting a patient for treatment with a MIF agonist or antagonist as described above.

For example, the MIF protein levels can be measured in a subject having a disease associated with high or low MIF expression and compared to the MIF protein levels in a subject that does not suffer from, or is not at risk for developing, a disease associated with high or low MIF expression. For example, MIF protein levels can be measured in a subject and compared to the MIF protein levels in a subject with a genotype that is associated with low MIF expression (e.g., a guanine at position −173 of both alleles of the MIF promoter and five CATT repeats in the −794 region of both alleles of the MIF promoter). Alternatively, MIF protein levels can be measured in a subject and compared to the MIF protein levels in a subject with a genotype that is associated with high MIF expression (e.g., a cytosine at position −173 of at least one of the alleles of the MIF promoter and/or six or more CATT repeats in the −794 region of at least one of the alleles of the MIF promoter).

Standard methods for measuring protein levels are known in the art. See, for example, Current Protocols in Molecular Biology, eds. Ausubel et al., John Wiley & Sons: 1992. For example, MIF protein levels can be measured by measuring the amount of light aborbance in a sample of the protein. Alternatively, MIF protein levels can be measured using an agent that binds to MIF protein, such as an antibody, an aptamer, a small molecule, another protein or an enzyme. Binding of the agent to MIF can be detected by the use of a signal (e.g., fluorescent, radioactive, chemical, or enzymatic), or may be detected by a chemical or enzymatic reaction. Other methods of measuring protein levels may include mass spectrometry, surface plasmon resonance or using protein chips.

### 4. Methods of Treating Diseases Associated with High or Low MIF Expression

In certain embodiments, the invention features methods of treating diseases associated with high or low MIF expression comprising administering to a subject in need thereof a therapeutically effective amount of a MIF agonist or a MIF antagonist. In one embodiment, the invention comprises administering to a subject having, or at risk of developing, a disease associated with high MIF expression a therapeutically effective amount of a MIF antagonist. In another embodiment, the invention comprises administering to a subject having, or at risk of developing, a disease associated with low MIF expression a therapeutically effective amount of a MIF agonist.

Diseases associated with high MIF expression include, without limitation, diseases caused by infection by a protozoan (for example malaria); anemia of chronic disease; and asthma.

Diseases associated with low MIF expression include, without limitation, any infection and the diseases caused by infections. In one embodiment, the infection is an acute infection. In one embodiment, the infection is a bacterial infection. In another embodiment, the infection is a viral infection. In another embodiment, the infection is a fungal infection. In one embodiment, the disease associated with low MIF expression is sepsis. In another embodiment, the disease associated with low MIF expression is an infection that leads to a respiratory disease (or a respiratory disease resulting from an infection), including without limitation, infections and diseases caused by gram positive and gram negative bacteria, mycobacteria (such as mycobacterium tuberculosis), fungal infections (e.g., infections of Pneumocystis, Candida, and Histoplasma) and viral infections (e.g., infections of influenza, varicella, and corona virus such as SARS-associated coronoavirus). In another embodiment, the disease associated with low MIF expression is meningitis. In another embodiment, the disease associated with low MIF expression is influenza. In one embodiment, the disease associated with low MIF expression is pneumonia (regardless of whether it is caused by a bacterial, viral or fungal infection). In a specific embodiment, the pneumonia is Community Acquired Pneumonia (CAP). In one embodiment, the viral infection is a retroviral infection. In one embodiment, the retroviral infection is HIV infection. In another embodiment, the disease associated with low MIF expression is infection by a virus or other pathogen that use the CCR5 receptor for infection, including, without limitation, HIV-1, HCV, Epstein-Barr Viruse, and Yersinia pestis.

MIF agonists and MIF antagonists can comprise, for example, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes. Agents may also comprise alcohols, alkyl halides, amines, amides, esters, aldehydes, ethers and other classes of organic agents. Exemplary MIF agonists and MIF antagonists are known in the art. Further, certain MIF agonists and MIF antagonists are described infra and supra.

The Use of MIF Antagonists to Treat Anemia of Chronic Disease

In one embodiment, the invention provides a method of treating anemia of chronic disease comprising administering to a subject a therapeutically effective amount of a MIF antagonist. In certain embodiment, the subject has or is at risk of developing anemia of chronic disease. In one embodiment, the subject has anemia of chronic disease and the subject is not responsive to erythropoietin (EPO) prior to the administration of the MIF antagonist. In one embodiment, the subject is has a genotype that is associated with high MIF expression. In one embodiment, the subject is Caucasian.

Anemia of chronic disease may result from, among other conditions, pathogenic infection (e.g., a malaria infection), cancer, autoimmune diseases or disorders, kidney diseases or disorders, organ transplant rejection and aging. The invention provides a method of treating anemia of chronic disease regardless of its cause.

The methods described herein may also comprise the administration of one or more other therapeutic agents. In certain embodiments, the invention provides a method of treating anemia of chromic disease comprising administering to a subject a therapeutically effective amount of a MIF antagonist in combination with one or more other agents that stimulate erythropoiesis. Examples of erythropoiesis-stimulating agents include, without limitation: erythropoietin (“EPO”), iron, folate, vitamin B12, blood, blood substitute, and plasma or serum that contains a composition with the activity of blood. In a specific embodiment, the invention provides a method of treating anemia of chromic disease, comprising administering to a subject in need thereof a MIF antagonist in combination with EPO.

In another embodiment, the invention provides a method of treating anemia of chronic disease, comprising administering to a subject a MIF antagonist in combination with a tumor necrosis factor-α (TNFα) antagonist or an interferon (IFN) antagonist (e.g., an IFNγ antagonist) to a subject. Examples of TNFα and IFNγ antagonists include, without limitation, anti-TNF, soluble TNF receptor, anti-IFNγ, soluble IFNγ receptor, p38 MAPK inhibitors, and JAK-STAT inhibitors.

The Use of MIF Antagonists to Malaria

The invention also comprises a method of treating malaria comprising administering to a subject in need thereof a MIF antagonist. In one embodiment, the subject has malaria or is at risk of developing malaria. In one embodiment, the subject is has a genotype that is associated with high MIF expression. In one embodiment, the subject is Caucasian.

The methods described herein may also comprise the administration of one or more other therapeutic agents.

The Use of MIF Agonists to Treat or Prevent Infections

The invention also comprises a method of treating an infection comprising administering to a subject a therapeutically effective amount of a MIF agonist. In one embodiment, the subject is has a genotype that is associated with low MIF expression.

Infections and diseases that are amenable to treatment with a MIF agonist include, without limitation, viral infections (including retroviral infections), bacterial infections, fungal infections, infections leading to respiratory disease, infections with HIV, pneumonia, Community Acquired Pneumonia (CAP), meningitis, and influenza. In certain embodiments, a MIF agonist is used to treat pathogenic infections during acute stages of infection, including during a flare-up of the infection, during a change of therapy, when signs of resistance to therapy are displayed in the subject, or as an early intervention.

In one embodiment, the invention provides a method of treating an infection that leads to a respiratory disease comprising administering to a subject a therapeutically effective amount of a MIF agonist. Infections that lead or may lead to respiratory disease include, without limitation, infections by gram positive and gram negative bacteria, mycobacteria (such as mycobacterium tuberculosis), fungal infections (e.g., infections of Pneumocystis, Candida, and Histoplasma) and viral infections (e.g., infections of influenza, varicella, and corona virus such as SARS-associated coronoavirus).

The invention also provides a method of treating a respiratory disease resulting from an infection comprising administering to a subject a therapeutically effective amount of a MIF agonist.

In certain embodiments, the invention provides a method of treating pneumonia in a subject comprising administering to the subject a therapeutically effective amount of a MIF agonist. Microbial infections that lead to pneumonia include, without limitation, bacterial infections (e.g., infections of gram positive bacteria, gram negative bacteria, and mycobacteria such as mycobacterium tuberculosis), fungal infections (e.g., infections of Pneumocystis, Candida, and Histoplasma) and viral infections (e.g., infections of influenza, varicella, and corona virus such as SARS-associated coronoavirus).

In certain embodiments, the invention provides a method of treating a retroviral infection comprising administering to a subject a therapeutically effective amount of a MIF agonist.

In certain embodiments, the invention provides a method of treating HIV infection comprising administering to a subject a therapeutically effective amount of a MIF agonist.

The invention also comprises the use of a MIF agonist as an immunoadjuvant.

The methods described herein may also comprise the administration of one or more other therapeutic agents, including without limitation anti-bacterial agents, anti-fungal agents and anti-microbial agents.

Examples of anti-viral agents include, without limitation, reverse transcriptase inhibitors such as, for example, zidovudine, didanosine, zalcitabine, stavudine, lamivudine, abacavir, nevirapine, delavirdine, and efavirenz; protease inhibitors such as, for example, saquinavir, ritonavir, nelfinavir, indinavir, amprenavir, and lopinavir; agents for treating herpes viruses such as, for example, acyclovir, valacyclovir, valacyclovir, famciclovir, ganciclovir, foscarnet, and cidolovir; and, agents for treating influenza such as, for example, oseltamivir, amantadine, rimatadine, and zanamivir. Examples of anti-bacterial agents include, without limitation, penicillins, cephalosporins, quinolones, tetracyclines, macrolides. Examples of anti-fungal agents include, without limitation, amphotericin, fluconozole.

Methods of Using a MIF Agonist to Attenuate Expression of CCR5 and Treat HIV Infection

In one embodiment, the invention provides a method of attenuating the expression of CCR5 mRNA or protein, comprising the use of a MIF agonist. For example, in one embodiment, cells expressing a CCR5 receptor are contacted with a MIF agonist wherein said contacting results in the attenuation of the expression of CCR5 mRNA or protein.

In another embodiment, the invention provides a method of inhibiting the life-cycle of a virus in a subject infected with said virus or at risk of being infected with said virus, wherein the virus uses the CCR5 as a receptor, administering to the subject a MIF agonist. In one embodiment, the pathogen that uses the CCR5 for infection is HIV-1.

As used herein the “inhibiting the life cycle of a virus” includes, inhibiting viral replication, inhibiting viral infection, latency and oncogenesis.

In a specific embodiment, the invention provides a method of treating HIV infection in a subject infected or at risk of being infected with HIV, comprising administering to the subject a MIF agonist. In one embodiment, the subject is has a genotype that is associated with low MIF expression. In certain embodiments, a MIF agonist is administered to a subject during acute HIV infection or during a flareup.

The methods described herein may also comprise the administration of one or more other therapeutic agents. In one embodiment, the methods described herein comprise the administration of a MIF agonist in combination with anti-viral agents. Examples of anti-viral agents include, without limitation, reverse transcriptase inhibitors such as, for example, zidovudine, didanosine, zalcitabine, stavudine, lamivudine, abacavir, nevirapine, delavirdine, and efavirenz; protease inhibitors such as, for example, saquinavir, ritonavir, nelfinavir, indinavir, amprenavir, and lopinavir; agents for treating herpes viruses such as, for example, acyclovir, valacyclovir, valacyclovir, famciclovir, ganciclovir, foscarnet, and cidolovir; and, agents for treating influenza such as, for example, oseltamivir, amantadine, rimatadine, and zanamivir.

### 5. Exemplary MIF Agonists and Antagonists

As described above any agent that mimics, activates, stimulates, potentiates or increases the biological activity of MIF can be used as a MIF agonist, and any agent that inhibits, opposes, counteracts, or decreases the biological activity of MIF can be used as a MIF antagonist.

MIF agonists include purified or recombinant nucleic acids that encode MIF proteins or fragments thereof; purified or recombinant MIF polypeptides or fragments thereof; or other agents that mimic, activate, stimulate, potentiate or increase the biological activity of MIF. Examples of MIF agonist include, without limitation, agents that increase MIF mRNA or protein expression; agents that enhance CD44 mRNA or protein expression; agents that enhance CD74 mRNA or protein expression; agents that increase interaction between MIF, CD74 and CD44 (e.g., bivalent antibodies that bind two out of three of MIF, CD74 and CD44, fusion proteins with CDR combinations that bind two out of three of MIF, CD74 and CD44, and other agents that are identified by any of the screening methods described herein); agents that increase interaction between CD44 and CD74 (e.g., bivalent antibodies that bind CD74 and CD44, fusion proteins with CDR combinations that bind CD74 and CD44, and other agents that are identified by any of the screening methods described herein); and agents that increase interaction between MIF and CD74 (e.g., bivalent antibodies that bind MIF and CD74, fusion proteins with CDR combinations that bind MIF and CD74, and other agents that are identified by any of the screening methods described herein).

In a preferred embodiment, the MIF agonist is recombinant MIF.

MIF antagonists include antibodies that bind to MIF (anti-MIF antibodies); small molecules that mimic MIF and inhibit MIF biological function (e.g., small molecules that bind CD74 and prevent MIF from binding CD74); agents that decrease or inhibit MIF mRNA or protein expression (e.g., antisense polynucleotides and RNAi); agents that decrease or inhibit CD74 mRNA or protein expression; agents that decrease or inhibit CD44 mRNA or protein expression; agents that prevent or decrease interaction between MIF, CD74 and CD44 (e.g., anti-MIF antibodies, anti-CD74 antibodies, anti-CD44 antibodies, or soluble fragments of CD74 or CD44); agents that prevent or decrease interaction between MIF and CD74 (e.g., anti-MIF antibodies, anti-CD74 antibodies, or soluble fragments of CD74); and agents that prevent or decrease interaction between CD74 and CD44 (e.g., anti-CD74 antibodies, anti-CD44 antibodies, or soluble fragments of CD74 or CD44).

Examples of MIF antagonists have been described in the art. See, e.g, Bernhagen et al., 1993. Nature 365, 756-759; Senter et al. 2002. Proc. Natl. Acad. Sci. USA 99, 144-149; Dios et al., 2002. J. Med. Chem. 45, 2410-2416; and Lubetsky et al., 2002. J. Biol. Chem. 277, 24976-24982. In a preferred embodiment, the MIF antagonist is COR100140 (a small molecule compound developed by Cortical Pty Ltd.), a small molecule compound developed by Avanir Pharmaceuticals, or an anti-MIF antibody.

In one embodiment, the D-dopachrome tautomerase (DDT) protein, or fragments thereof, is used to modulate (e.g., agonize or antagonize) the biogical activity of MIF. In another embodiment, an agonist or antagonist of DDT is used to modulate (agonize or antagonize) the activity of MIF. An exemplary nucleotide sequence that encodes DDT is found in GenBank Accession No. AH006997.

CD44-Dependent Modulation of MIF Activity

A cell surface receptor for MIF was cloned in 2003 and identified to be the widely-expressed, Type II transmembrane protein, CD74 (Leng et al. (2003). J Exp Med 197:1467-1476). The data described herein (Example 6) demonstrates that CD44 is also required for MIF-mediated ERK-1/2 activity.

Accordingly, the present invention provides novel agonists and antagonists of MIF that modulate the interaction between MIF, CD74 and CD44. The invention also provides methods of agonizing or antagonizing the biological function of MIF, comprising the use of agents that modulate the interaction between MIF, CD74 and CD44. Agents that may be used in the invention include, without limitation, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes. Agents may also comprise alcohols, alkyl halides, amines, amides, esters, aldehydes, ethers and other classes of organic agents.

In one embodiment, the invention provides a method of attenuating (agonizing or antagonizing) the biological function of MIF, comprising the use of an agent that inhibits the interaction between CD44 and CD74. In certain embodiments, the agent is a fragment or an extracellular fragment of CD44. In other embodiments, the agent is an agent that binds to CD44. In one embodiment, the agent is an antibody or fragment thereof that binds to CD44. In other embodiments, the agent is a small molecule, such as a small molecule mimic of chondroitin sulphate. In another embodiment, the agent is heparin. In another embodiment, the agent is a macromolecular mimic of chondroitin sulphate.

In another embodiment, the invention provides a method of attenuating the biological function of MIF, comprising the use of an agent that inhibits or decreases the expression of CD44. Agents that may be useful for inhibiting or decreasing the expression of CD44 include, for example, siRNAs (or other antisense polynucleotide) that target CD44 mRNA.

In other embodiments, the invention provides a method of attenuating the biological function of MIF, comprising the use of an agent that inhibits or decreases the activation of a src family tyrosine kinase (e.g., p561ck). Agents that may be useful for inhibiting or decreasing the activation of src family tyrosine kinases include, for example, siRNAs that target src family tyrosine kinase mRNAs (e.g., p561ck mRNA) or small molecules such as, for example, damnacanthal. Other agents that may be useful for inhibiting the activation of a src family tyrosine kinase include, for example, agents that inhibit PKA phosphorylation or agents that antagonize the activity of a src tyrosine kinase family member.

In another embodiment, the invention provides a method of enhancing or increasing the biological function of MIF, comprising the use of an agent that enhances the interaction between MIF, CD44 and CD74. Agents that may be useful for enhancing the interaction of MIF, CD44 and CD74 include, for example, antibodies and fusion proteins that bind to CD44 and CD74 and antibodies and fusion protein that bind to MIF and CD44. In a specific embodiment, an agent that enhances the interaction between MIF, CD44 and CD74 is an anti-MIF and anti-CD74 bivalent antibody, an anti-CD74 and anti-CD44 bivalent antibody, or an anti-MIF and anti-CD44 bivalent antibody. In other embodiments, an agent that enhances the interaction between MIF, CD44 and CD74 is a recombinant fusion protein that comprises the complementarity determining regions (CDRs) of anti-MIF and anti-CD74, anti-CD74 and anti-CD44, or anti-MIF and anti-CD44 antibodies, or of an anti-MIF, anti-CD74 and anti-CD44 antibody.

Antisense Polynucleotides

Antisense polynucleotides can also be used as MIF agonist or MIF antagonists. In certain embodiments, the invention provides polynucleotides that comprise an antisense sequence that acts through an antisense mechanism for inhibiting expression of a MIF gene or a gene encoding a protein that affects MIF expression or MIF biological function (including, without limitation CD44 and CD74).

In one embodiment, a MIF antagonist may be an antisense polynucleotide that binds to a MIF gene and decreases expression of said MIF gene. In another embodiment, a MIF antagonist may be an antisense polynucleotide that binds to a CD44 gene and decreases expression of said CD44 gene. In another embodiment, a MIF antagonist may be an antisense polynucleotide that binds to a CD74 gene and decreases expression of said CD74 gene.

Antisense technologies have been widely utilized to regulate gene expression (Buskirk et al., Chem. Biol. 11, 1157-63 (2004); and Weiss et al., Cell Mod. Life Sci. 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hinderance, altering splicing, or inducing cleavage or other enzymatic inactivation of the transcript. The binding may be by conventional base pair complementarity, or, for example, in the case of binding to DNA duplexes, through specific interactions in the major groove of the double helix. In general, “antisense” technology refers to the range of techniques generally employed in the art, and includes any therapy that relies on specific binding to nucleic acid sequences.

A polynucleotide that comprises an antisense sequence of the present invention can be delivered, for example, as a component of an expression plasmid which, when transcribed in the cell, produces a nucleic acid sequence that is complimentary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention may be modified so that they are resistant to endogenous nucleases, e.g. exonucleases and/or endonucleases, and are therefore stable in vivo. Examples of nucleic acid molecules for use in polynucleotides of the invention are phosphoramidate, phosphothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der krol et al. (1988) Biotechniques 6:958-976; and Stein et al. (1988) Cancer Res. 48:2659-2668.

Antisense approaches involve the design of polynucleotides (either DNA or RNA) that are complementary to a target nucleic acid that modulates MIF biological function. For example, antisense polynucleotides complementary to nucleic acids that encode MIF, a protein that regulates MIF, or a protein that is important for the biological function of MIF (e.g., CD74 and CD44) may modulate MIF biological function. The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have recently been shown to be effective at inhibiting translation of mRNAs as well (Wagner, R. 1994. Nature 372:333). Therefore, antisense polynucleotides complementary to either the 5′ or 3′ untranslated, non-coding regions of a gene that modulates the biological function of MIF (e.g., a nucleic acid that encodes MIF, a protein that regulates MIF, or a protein that is important for the biological function of MIF such as CD74 and CD44) could be used in an antisense approach to inhibit translation of a protein that modulates the biological function of MIF. Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense polynucleotide to inhibit expression of a gene that modulates the biological function of MIF (e.g., a nucleic acid that encodes MIF, a protein that regulates MIF, or a protein that is important for the biological function of MIF such as CD74 and CD44). It is preferred that these studies utilize controls that distinguish between antisense gene inhibition and nonspecific biological effects of antisense polynucleotide. It is also preferred that these studies compare levels of the target RNA or protein with that of an internal control RNA or protein. Additionally, it is envisioned that results obtained using the antisense polynucleotide are compared with those obtained using a control antisense polynucleotide. It is preferred that the control antisense polynucleotide is of approximately the same length as the test antisense polynucleotide and that the nucleotide sequence of the control antisense polynucleotide differs from the antisense sequence of interest no more than is necessary to prevent specific hybridization to the target sequence.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad Sci. USA 86:6553-6556; Lemaitre et al., 1987, Proc Natl Acad Sci. USA 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

Polynucleotides of the invention may also comprise at least one modified sugar moiety selected from the group including but not limited to arabinose, 2-fluoroarabinose, xylulose, and hexose.

A polynucleotide of the invention can also contain a neutral peptide-like backbone. Such molecules are termed peptide nucleic acid (PNA)-oligomers and are described, e.g., in Perry-O′Keefe et al. (1996) Proc. Natl. Acad. Sci. USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a PCT/US2006/016254 phosphorodithioate, a phosphoramidothioate, a phosphoramidate, a phosphordiamidate, a methylphosphonate, an alkyl phosphotriester, and a formacetal or analog thereof.

In a further embodiment, polynucleotides of the invention, including antisense polynucleotides are -anomeric oligonucleotides. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc. Natl. Acad. Sci. USA 85:7448-7451 (1988)), etc.

While antisense sequences complementary to the coding region of an mRNA sequence can be used, those complementary to the transcribed untranslated region and to the region comprising the initiating methionine are most preferred.

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

However, it may be difficult to achieve intracellular concentrations of the antisense polynucleotides sufficient to attenuate the activity of a specific gene or mRNA in certain instances. Therefore, another approach utilizes a recombinant DNA construct in which the antisense polynucleotide is placed under the control of a strong pol III or pol II promoter. The use of such a construct to transfect target cells in the patient will result in the transcription of sufficient amounts of antisense polynucleotides to attenuate the activity of the targeted gene or protein. For example, a vector can be introduced in vivo such that it is taken up by a cell and directs the transcription of an antisense polynucleotide that targets a gene that modulates the biological function of MIF (e.g., a nucleic acid that encodes MIF, a protein that regulates MIF, or a protein that is important for the biological function of Mae such as CD74 and CD44). Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionine gene (Brinster et al, Nature 296:3942 (1982)), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi Constructs—siRNAs and miRNAs

RNAi molecules can also be used as MIF agonist or MIF antagonists. Accordingly, the present invention provides a polynucleotide comprising an RNAi sequence that acts through an RNAi or miRNA mechanism to attenuate expression of a MIF gene or a gene encoding a protein that affects MIF expression or MIF biological function. Accordingly, RNAi polynucleotides can act as MIF agonists or MIF antagonists.

In one embodiment, a MIF antagonist may be an RNAi polynucleotide that binds to a MIF gene and decreases expression of said MIF gene. In another embodiment, a MIF antagonist may be an RNAi polynucleotide that binds to a CD44 gene and decreases expression of said CD44 gene. In another embodiment, a MIF antagonist may be an RNAi polynucleotide that binds to a CD74 gene and decreases expression of said CD74 gene. In one embodiment, the miRNA or siRNA sequence is between about 19 nucleotides and about 75 nucleotides in length, or preferably, between about 25 base pairs and about 35 base pairs in length. In certain embodiments, the polynucleotide is a hairpin loop or stem-loop that may be processed by RNAse enzymes (e.g., Drosha and Dicer).

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs could mediate gene specific RNAi in mammalian cells, without invoking generic antiviral defense mechanisms. Elbashir et al. Nature 2001, 411:494-498; Caplen et al. Proc Natl Acad Sci 2001, 98:9742-9747. As a result, small-interfering RNAs (siRNAs) and micro RNAs (miRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

An RNAi construct contains a nucleotide sequence that hybridizes under physiologic conditions of the cell to the nucleotide sequence of at least a portion of the mRNA transcript of a gene that modulates the biological function of MIF (e.g., a nucleic acid that encodes MIF, a protein that regulates MIF, or a protein that is important for the biological function of MIF such as CD74 and CD44). The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important that the RNAi construct is able to specifically target a MIF gene, or a gene important for the biological function of MIF. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of polynucleotides comprising RNAi sequences can be carried out by any of the methods for producing polynucleotides described herein. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res., 25:776-780; Wilson et al. (1994) J. Mol. Recog. 7:89-98; Chen et al. (1995) Nucleic Acids Res. 23:2661-2668; Hirschbein et al. (1997) Antisense Nucleic Acid Drug Dev. 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse III-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs. (Brennecke et al., Genome Biology 4:228 (2003); Kim et al., Mol. Cells 19:1-15 (2005).)

In certain embodiments, miRNA and siRNA constructs can be generated by processing of longer double-stranded RNAs, for example, in the presence of the enzymes Dicer or Drosha. Dicer and Drosha are RNAse III-like nucleases that specifically cleave dsRNA. Dicer has a distinctive structure which includes a helicase domain and dual RNAse III motifs. Dicer also contains a region of homology to the RDE1/QDE2/ARGONAUTE family, which have been genetically linked to RNAi in lower eukaryotes. Indeed, activation of, or overexpression of Dicer may be sufficient in many cases to permit RNA interference in otherwise non-receptive cells, such as cultured eukaryotic cells, or mammalian (non-oocytic) cells in culture or in whole organisms. Methods and compositions employing Dicer, as well as other RNAi enzymes, are described in U.S. Pat. App. Publication No. 2004/0086884.

In one embodiment, the Drosophila in vitro system is used. In this embodiment, a polynucleotide comprising an RNAi sequence or an RNAi precursor is combined with a soluble extract derived from Drosophila embryo, thereby producing a combination. The combination is maintained under conditions in which the dsRNA is processed to RNA molecules of about 21 to about 23 nucleotides.

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev., 2002, 16:948-58; McCaffrey et al., Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA.

Aptamers and Small Molecules

Aptamers can also be used as MIF agonist or MIF antagonists. The present invention provides therapeutic aptamers that specifically bind to a MIF polypeptide or a polypeptide that affects MIF expression or MIF biological function, thereby modulating (e.g., agonizing or antagonizing) activity of MIF.

In one embodiment, a MIF agonist may be an aptamer that binds to a MIF polypeptide and activates, stimulates or potentiates the activity of said MIF polypeptide. In one embodiment, a MIF agonist may be an aptamer that binds to a CD44 polypeptide and activates, stimulates or potentiates the activity of said CD44 polypeptide. In one embodiment, a MIF agonist may be an aptamer that binds to a CD74 polypeptide and activates, stimulates or potentiates the activity of said CD74 polypeptide.

In another embodiment, a MIF antagonist may be an aptamer that binds to a MIF polypeptide and attenuates, inhibits, opposes, counteracts, or decreases the activity of said MIF polypeptide. In one embodiment, a MIF antagonist may be an aptamer that binds to a CD44 polypeptide and attenuates, inhibits, opposes, counteracts, or decreases the activity of said CD44 polypeptide. In one embodiment, a MIF agonist may be an aptamer that binds to a CD74 polypeptide and attenuates, inhibits, opposes, counteracts, or decreases the activity of said CD74 polypeptide.

An “aptamer” may be a nucleic acid molecule, such as RNA or DNA that is capable of binding to a specific molecule with high affinity and specificity (Ellington et al., Nature 346, 818-22 (1990); and Tuerk et al., Science 249, 505-10 (1990)). An aptamer will most typically have been obtained by in vitro selection for binding of a target molecule. For example, an aptamer that specifically binds to polypeptide important for the biological function of MIF (e.g., MIF, CD74 or CD44) can be obtained by in vitro selection from a pool of polynucleotides for binding to a polypeptide important for the biological function of MIF (e.g., MIF, CD74 or CD44). However, in vivo selection of an aptamer is also possible. Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand (e.g., a MIF polypeptide, or a polypeptide important for the biological function of MIF such as CD74 or CD44) as compared to the dissociation constant of the aptamer for other materials in the environment or unrelated molecules in general. A ligand (e.g., a MIF, CD74 or CD44 polypeptide) is one which binds to the aptamer with greater affinity than to unrelated material. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 300 nucleotides in length. More commonly, an aptamer will be between about 30 and about 100 nucleotides in length.

Methods for selecting aptamers specific for a target of interest are known in the art. For example, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have been successfully used as target ligands for in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see Famulok, Science 9:324-9 (1999).

An aptamer of the invention can be comprised entirely of RNA. In other embodiments of the invention, however, the aptamer can instead be comprised entirely of DNA, or partially of DNA, or partially of other nucleotide analogs. To specifically inhibit translation in vivo, RNA aptamers are preferred. Such RNA aptamers are preferably introduced into a cell as DNA that is transcribed into the RNA aptamer. Alternatively, an RNA aptamer itself can be introduced into a cell.

Aptamers are typically developed to bind particular ligands by employing known in vivo or in vitro (most typically, in vitro) selection techniques known as SELEX (Ellington et al., Nature 346, 818-22 (1990); and Tuerk et al., Science 249, 505-10 (1990)). Methods of making aptamers are also described in, for example, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Generally, in their most basic form, in vitro selection techniques for identifying aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques, although any means that will allow faithful, efficient amplification of selected nucleic acid sequences can be employed. The DNA pool is then in vitro transcribed to produce RNA transcripts. The RNA transcripts may then be subjected to affinity chromatography, although any protocol which will allow selection of nucleic acids based on their ability to bind specifically to another molecule (e.g., a protein or any target molecule) may be used. In the case of affinity chromatography, the transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand. Once an aptamer sequence has been successfully identified, the aptamer may be further optimized by performing additional rounds of selection starting from a pool of oligonucleotides comprising the mutagenized aptamer sequence. For use in the present invention, the aptamer is preferably selected for ligand binding in the presence of salt concentrations and temperatures which mimic normal physiological conditions.

The unique nature of the in vitro selection process allows for the isolation of a suitable aptamer that binds a desired ligand despite a complete dearth of prior knowledge as to what type of structure might bind the desired ligand.

The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

The present invention also provides small molecules and antibodies that specifically bind to a polypeptide important for the biological function of MIF (e.g., MIF, CD74 or CD44), thereby modulating (agonizing or antagonizing) the biological function of MIF. Examples of small molecules include, without limitation, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes).

Antibodies, Antibody Fragments and Other Fusion Proteins

Antibodies or fragments thereof specifically reactive with a polypeptide that affects the expression or biological function of MIF may be used as MIF agonists or MIF antagonists. Antibodies or fragments thereof directed, for example, to MIF, CD44, CD74, and/or combinations thereof, may be agonists or antagonists of the expression or biological function of MIF.

Antibodies or fragments thereof can also be used to detect MIF or a protein important for the biological function of MIF (e.g., CD74 or CD44). In one embodiment, an antibody or fragment thereof that is specifically reactive with a MIF polypeptide may be used to detect the presence of a MIF polypeptide. In another embodiment, an antibody or fragment thereof that is specifically reactive with a CD74 polypeptide may be used to detect the presence of a CD74 polypeptide. In another embodiment, an antibody or fragment thereof that is specifically reactive with a CD44 polypeptide may be used to detect the presence of a CD44 polypeptide.

In another embodiment, an antibody or a fragment thereof that is specifically reactive with a MIF polypeptide may be used as a MIF antagonist to inhibit the activity of a MIF polypeptide. In another embodiment, an antibody or fragment thereof that is specifically reactive with CD44 may be used as a MIF antagonist to inhibit the biological function of MIF. In another embodiment, an antibody or fragment thereof that is specifically reactive with CD74 may be used as a MIF antagonist to inhibit the biological function of MIF.

In another embodiment, an antibody or fragment thereof that is specifically reactive with a MIF polypeptide may be used as a MIF agonist to increase or activate the activity or a MIF polypeptide. In one embodiment, a MIF agonist is an antibody, such as a bivalent antibody or a fragment thereof, that is able to bind MIF. In another embodiment, a MIF agonist is be an antibody, such as a bivalent antibody or a fragment thereof, that is able to bind MIF and CD44. In another embodiment, a MIF agonist is be an antibody, such as a bivalent antibody or a fragment thereof, that is able to CD44 and CD74.

Methods of making antibodies are well known in the art. For example, by using immunogens derived from a MIF polypeptide or a polypeptide that affects the expression or biological function of MIF, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the MIF polypeptide, an antigenic fragment which is capable of eliciting an antibody response, or a fusion protein. Such mammals can also be immunized with an immunogenic form of a polypeptide that affects the expression or biological function of MIF. Techniques for conferring immunogenicity on a protein or peptide include conjugation to carriers or other techniques well known in the art. An immunogenic portion of a MIF polypeptide or a polypeptide that affects the expression or biological function of MIF can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a MIF polypeptide or a polypeptide that affects the expression or biological function of MIF, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a MIF polypeptide or a polypeptide that affects the expression or biological function of MIF. Monoclonal antibodies can be isolated from a culture comprising such hybridoma cells.

The term “antibody” as used herein is intended to include fragments thereof which are also specifically reactive with a MIF polypeptide or a polypeptide that affects the expression or biological function of MIF. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)2, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that bind specifically to MIF polypeptides or to polypeptides that affect the expression or biological function of MIF. For example, a method for generating a monoclonal antibody that binds specifically to a MIF polypeptide, or to a polypeptide that affects the expression or biological function of MIF, may comprise administering to a mouse an amount of an immunogenic composition comprising the MIF polypeptide or the polypeptide that affects the expression or biological function of MIF, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the MIF polypeptide or the polypeptide that affects the expression or biological function of MIF. Once obtained, a hybridoma can be propagated in a cell culture, optionally in culture conditions where the hybridoma-derived cells produce the monoclonal antibody that binds specifically to the MIF polypeptide or the polypeptide that affects the expression or biological function of MIF. The monoclonal antibody may be purified from the cell culture.

The term “specifically reactive with” as used in reference to an antibody is intended to mean, as is generally understood in the art, that the antibody is sufficiently selective between the antigen of interest (e.g., a MIF polypeptide or a polypeptide that affects the expression or biological function of MIF) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc., Gaithersburg, Md.), western blots, immunoprecipitation assays, and immunohistochemistry.

### 6. General Information Relating to Methods of Treatment Using MIF Agonists or MIF Antagonist

The methods described herein for treating a subject suffering from or at risk of developing a disease or condition associated with high or low MIF expression may be used for the prophylactic treatment of individuals who have been diagnosed or predicted to be at risk for developing a disease or condition associated with high or low MIF expression. Thus, in one embodiment, a composition comprising a MIF agonist or antagonist is administered in an amount and dose that is sufficient to delay, slow, or prevent the onset of a disease or condition associated with high or low MIF expression, or related symptoms, or to reverse a disease or condition associated with high or low MIF expression. It is understood that an effective amount of a composition for treating a subject who has been diagnosed or predicted to be at risk for developing a disease or condition associated with high or low MIF expression is a dose or amount that is in sufficient quantities to treat a subject or to treat the disorder itself.

MIF agonists and antagonists may be formulated with a pharmaceutically acceptable carrier. For example, a MIF agonist or antagonist can be administered alone or as a component of a pharmaceutical formulation (therapeutic composition). The MIF agonist or antagonist may be formulated for administration in any convenient way for use in human medicine.

In certain embodiments, the therapeutic methods of the invention include administering the composition topically, systemically, or locally. For example, therapeutic compositions of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. The compositions described herein may be formulated as part of an implant or device. When administered, the therapeutic composition for use in this invention is in a pyrogen-free, physiologically acceptable form. Further, the composition may be encapsulated or injected in a viscous form for delivery to the site where the target cells are present. Techniques and formulations generally may be found in Remington's Pharmaceutical Sciences, Meade Publishing Co., Easton, Pa. In addition to MIF agonists or antagonists, therapeutically useful agents may optionally be included in any of the compositions described herein. Furthermore, therapeutically useful agents may, alternatively or additionally, be administered simultaneously or sequentially with a MIF agonist or antagonist according to the methods of the invention.

In certain embodiments, compositions comprising a MIF agonist or antagonist can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more compositions comprising a MIF agonist or antagonist may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required. The ointments, pastes, creams and gels may contain, in addition to a MIF agonist or antagonist, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a MIF agonist or antagonist, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, pharmaceutical compositions suitable for parenteral administration may comprise a MIF agonist or antagonist in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A composition comprising a MIF agonist or antagonist may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments, the present invention also provides gene therapy for the in vivo production of a MIF agonist or antagonist. Such therapy would achieve its therapeutic effect by introduction of a polynucleotide sequence that encodes a MIF agonist or antagonist into cells or tissues that are deficient for normal MIF function. Delivery of MIF agonist or antagonist polynucleotide sequences can be achieved using a recombinant expression vector such as a chimeric virus or a colloidal dispersion system. Targeted liposomes may also be used for the therapeutic delivery of CFH polynucleotide sequences.

Various viral vectors which can be utilized for gene therapy as taught herein include adenovirus, herpes virus, vaccinia, or an RNA virus such as a retrovirus. A retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the polynucleotide that encodes the MIF agonist or antagonist.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for polynucleotides that encode MIF agonists or antagonists is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see, e.g., Fraley, et al., Trends Biochem. Sci. 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

Moreover, a composition comprising a MIF agonist or antagonist can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinacious biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

A person of ordinary skill in the art, such as a physician, is able to determine the required amount to treat the subject. The amount of a MIF agonist or antagonist administered to effectively treat a disease or condition associated with high or low MIF expression is an amount that significantly decreases or inhibits any symptom associated with the disease or condition. It is understood that the dosage regimen will be determined for an individual, taking into consideration, for example, various factors that modify the action of a MIF agonist or antagonist, the severity or stage of the disease or condition associated with high or low MIF expression, route of administration, and characteristics unique to the individual, such as age, weight, and size. In one embodiment, the dosage can range from about 1.0 ng/kg to about 100 mg/kg body weight of the subject.

In certain embodiments, a composition comprising a MIF agonist or antagonist for topical, systemic or local administration can be administered in a range from about 0.001% to about 3.0% (weight per volume or weight per weight), or from about 0.001% to about 0.01%, from about 0.01% to about 0.025%, from about 0.025% to about 0.05%, from about 0.05% to about 0.1%, from about 0.1% to about 0.25%, from about 0.25% to about 1.0%, from about 1.0% to about 2.0%, or from about 2.0% to greater than 3.0%, i.e., about 3.0% to about 10.0% or greater. In a specific embodiment, a composition comprising a MIF agonist or antagonist is administered in a range from about 0.25% to about 3.0%.

In certain embodiments, a composition comprising a MIF agonist or antagonist is administered in a range of from about 1 ng/ml to about 1 g/ml, or from about 1 ng/ml to about 10 ng/ml, from about 10 ng/ml to about 100 ng/ml, from about 100 ng/ml to about 1 mg/ml, from about 1 mg/ml to about 10 mg/ml, from about 10 mg/ml to about 100 mg/ml or from about 100 mg/ml to about 1 g/ml. In certain embodiments, a composition comprising a MIF agonist or antagonist is administered in a range of from about 40 ng/ml to about 100 ng/ml.

The volume of composition administered according to the methods described herein is also dependent on factors such as the mode of administration, quantity of the MIF agonist or antagonist, age and weight of the patient, and type and severity of the disease being treated. For example, if administered orally as a liquid, the liquid volume comprising a composition comprising a MIF agonist or antagonist may be from about 0.5 milliliters to about 2.0 milliliters, from about 2.0 milliliters to about 5.0 milliliters, from about 5.0 milliliters to about 10.0 milliliters, or from about 10.0 milliliters to about 50.0 milliliters. If administered by injection, the liquid volume comprising a composition comprising a MIF agonist or antagonist may be from about 5.0 microliters to about 50 microliters, from about 50 microliters to about 250 microliters, from about 250 microliters to about 1 milliliter, from about 1 milliliter to about 5 milliliters, from about 5 milliliters to about 25 milliliters, from about 25 milliliters to about 100 milliliters, or from about 100 milliliters to about 1 liter.

The dose can be delivered continuously, or at periodic intervals (e.g., on one or more separate occasions). Desired time intervals of multiple doses of a particular composition can be determined without undue experimentation by one skilled in the art. For example, the compound may be delivered hourly, daily, weekly, monthly, yearly (e.g. in a time release form) or as a one time delivery. If administered orally or topically, such a preparation can be administered 1 to 6 times per day for a period of 1-4 weeks, 1-3 months, 3-6 months, 6-12 months, 1-2 years, or more, up to the lifetime of the patient. If administered by injection, MIF agonist or antagonist compositions can be delivered one or more times periodically throughout the life of a patient. For example, a MIF agonist or antagonist composition can be delivered once per year, once every 6-12 months, once every 3-6 months, once every 1-3 months, once every 1-4 weeks, one or more times per day. Alternatively, more frequent administration may be desirable for certain conditions or disorders. If administered by an implant or device, MIF agonist or antagonist compositions can be administered one time, or one or more times periodically throughout the lifetime of the patient as necessary.

Samples used in the methods described herein may comprise cells from the eye, ear, nose, throat, teeth, tongue, epidermis, epithelium, blood, tears, saliva, mucus, urinary tract, urine, muscle, cartilage, skin, or any other tissue or bodily fluid from which sufficient DNA, RNA or protein can be obtained. In certain embodiments, samples used in the methods described herein comprise cells from a tracheal aspirate or nasal washing.

The sample should be sufficiently processed to render the DNA, RNA or protein that is present in the sample available for assaying in the methods described herein. For example, samples may be processed such that DNA from the sample is available for amplification or for hybridization to another polynucleotide. The processed samples may be crude lysates where available DNA, RNA or protein is not purified from other cellular material. Alternatively, samples may be processed to isolate the available DNA, RNA or protein from one or more contaminants that are present in its natural source. Samples may be processed by any means known in the art that renders DNA, RNA or protein available for assaying in the methods described herein. Methods for processing samples may include, without limitation, mechanical, chemical, or molecular means of lysing and/or purifying cells and cell lysates. Processing methods may include, for example, ion-exchange chromatography, size exclusion chromatography, affinity chromatography, hydrophobic interaction chromatography, gel filtration chromatography, ultrafiltration, electrophoresis, and immunoaffinity purification with antibodies specific for particular epitopes of the polypeptide

### 7. Screening Assays

In certain aspects, the present invention relates to the use of MIF, CD44 and/or CD74 to identify agents that are agonists or antagonists of the biological function of MIF. Agents identified through this screening can be tested in cells and tissues to assess their ability to modulate the biological activity of MIF in vivo or in vitro. In certain aspects, these agents can further be tested in animal models to assess their ability to modulate the biological activity of MIF in vivo. The compounds identified in these methods can be used to treat diseases associated with high or low MIF expression as described herein. The methods described herein are based on the discovery that. CD44 functionally interacts with MIF (Meyer-Siegler et al., BMC Cancer, 4:34 (2004) and Meyer-Siegler et al., J Urol., 173:615-620 (2005)).

In one embodiment, the invention provides a method of identifying potential agonists or antagonists of the biological function of MIF, comprising: (a) contacting a CD44 polypeptide, or a portion thereof, with a CD74 polypeptide, or portion thereof, in the presence and absence of a candidate agent; and (b) comparing the interaction of the CD44 and CD74 polypeptides in the presence of said candidate agent with the interaction in the absence of said candidate agent. A candidate agent that enhances the interaction of the CD44 polypeptide and the CD74 polypeptide is thus identified as a potential agonist of MIF biological function, and a candidate agent that inhibits the interaction of the CD44 polypeptide and the CD74 polypeptide is identified as a potential antagonist of MIF biological function.

In another embodiment, the invention provides a method of identifying potential agonists or antagonists of the biological function of MIF, comprising: (a) contacting a CD44 polypeptide or a portion thereof, with a MIF polypeptide, or a portion thereof, and a CD74 polypeptide or a portion thereof, in the presence and absence of a candidate agent; and, (b) comparing the interaction of the CD44 polypeptide or portion thereof and the MIF and CD74 polypeptides or portions thereof in the presence of said candidate agent with the interaction in the absence of said candidate agent. A candidate agent that enhances the interaction of the CD44 polypeptide and the MIF and CD74 polypeptides is thus identified as a potential agonist of MIF biological function, and a candidate agent that inhibits the interaction of the CD44 polypeptide and the MIF and CD74 polypeptides is identified as a potential antagonist of MIF biological function.

The interaction between the agent and the subject polypeptide (e.g., CD44, CD74, MIF, and/or MIF/CD74) may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46:1). In certain cases, the agents may be screened in a mechanism based assay, such as an assay to detect agents which bind to the subject polypeptide (e.g., CD44, CD74, MIF, and/or MIF/CD74). This may include a solid phase or fluid phase binding event. Alternatively, the gene or genes encoding one or more of the subject polypeptides can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound agents may be detected usually using colorimetric or fluorescence or surface plasmon resonance.

In certain embodiments, high-throughput screening of agents can be carried out to identify agents that affect the biological function of MIF. A variety of assay formats will suffice and, in light of the present disclosure, those not expressly described herein will nevertheless be comprehended by one of ordinary skill in the art. As described herein, the test agents of the invention may be created by any combinatorial chemical method. Alternatively, the subject agents may be naturally occurring biomolecules synthesized in vivo or in vitro. Agents to be tested for their ability to act as modulators of the biological function of MIF can be produced, for example, by bacteria, yeast, plants or other organisms (e.g., natural products), produced chemically (e.g., small molecules, including peptidomimetics), or produced recombinantly. Test agents contemplated by the present invention include non-peptidyl organic molecules, peptides, polypeptides, polysaccharides, peptidomimetics, sugars, hormones, and nucleic acid molecules.

The candidate agents of the invention can be provided as single, discrete entities, or provided in libraries of greater complexity, such as made by combinatorial chemistry. The agents can comprise, for example, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes. Agents may also comprise alcohols, alkyl halides, amines, amides, esters, aldehydes, ethers and other classes of organic agents. Presentation of candidate agents to the test system can be in either an isolated form or as mixtures of agents, especially in initial screening steps. Optionally, the agents may be derivatized with other agents and have derivatizing groups that facilitate isolation of the agents. Non-limiting examples of derivatizing groups include biotin, fluorescein, digoxygenin, green fluorescent protein, isotopes, polyhistidine, magnetic beads, glutathione S transferase (GST), photoactivatible crosslinkers or any combinations thereof.

In many screening programs which test libraries of agents and natural extracts, high throughput assays are desirable in order to maximize the number of agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test agent. Moreover, the effects of cellular toxicity or bioavailability of the test agent can be generally ignored in the in vitro system, the assay instead being focused primarily on the effect of the agent on the molecular target.

### 6. Kits

Also provided herein are kits, e.g., kits for therapeutic purposes or kits for: (i) diagnosing a patient for a disease associated with high or low MIF expression, (ii) identifying patients at risk of developing a disease associated with high or low MIF expression, (iii) predicting the severity of a disease associated with high or low MIF expression, (iv) predicting the susceptibility of a patient to a disease associated with high or low MIF expression, (v) selecting a patient for treatment with a MIF agonist or antagonist, and (vi) genotyping a patient for the presence of a polymorphism associated with high or low MIF expression. In one embodiment, a kit comprises at least one container means having disposed therein reagents for genotyping a subject for the presence of a polymorphism associated with high or low MIF expression. For illustrative purposes, genotyping reagents may include polynucleotide probes or primers, or solid substrates such as chips or microarrays that are capable of detecting whether a polymorphism associated with high or low MIF expression is present. For use in a kit, polynucleotides may be any of a variety of natural and/or synthetic compositions, or chimeric mixtures thereof, such as synthetic polynucleotides, restriction fragments, cDNAs, synthetic peptide nucleic acids (PNAs), and the like. The assay kit and method may also employ labeled polynucleotides to allow ease of identification in the assays. Examples of labels which may be employed include radiolabels, enzymes, fluorescent compounds, streptavidin, avidin, biotin, magnetic moieties, metal binding moieties, antigen, enzymatic or antibody moieties, and the like. The kit may optionally comprise a label and/or instructions for use.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dematol. 103:387-389 (1994)) and the like; DNA purification reagents such as Nucleon™ kits, lysis buffers, proteinase solutions and the like; PCR reagents, such as 10× reaction buffers, thermostable polymerase, dNTPs, and the like; and allele detection means such as the Hint/restriction enzyme, allele specific oligonucleotides, degenerate oligonucleotide primers for nested PCR from dried blood. Other kit reagents may include enzymes, buffers, small molecules, nucleotides or their analogs, labels (e.g., fluorescent, radioactive, enzymatic or chemical) and/or co-factors as required for the genotyping assay.

In another embodiment, a kit comprises at least one container means having disposed therein a premeasured dose of one or more MIF antagonists and/or MIF agonists. A kit may optionally comprise devices for contacting cells with the MIF antagonists and/or MIF agonists and a label and/or instructions for use. Devices include syringes, dispensers, stents and other devices for introducing a MIF antagonist and/or MIF agonist into a subject (e.g., the blood vessel of a subject) or applying it to the skin of a subject. Kits may also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and, Handbook Of Experimental Immunology, Volumes I-IV (D. M. Weir and C. C. Blackwell, eds., 1986).

### EXAMPLES

The following examples are for illustrative purposes and are not intended to be limiting in any way.

### Example 1

### MIF in the Pathogenesis of Anemia of Chronic Disease and Malaria

Effect of MIF and Pro-Inflammatory Cytokines on Erythropoiesis.

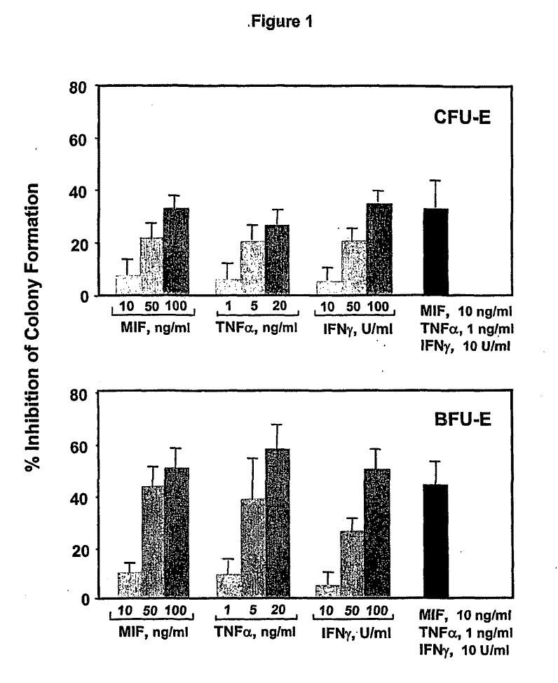

The effect of MIF on murine erythroid precursor development was examined using defined, erythropoietin-dependent colony assays, including both the early, burst forming unit-erythroid (BFU-E), and the late, colony forming unit-erythroid (CFU-E). The two inflammatory cytokines, TNFα and IFNγ, were also studied. TNFα and IFNγ are produced as a consequence of macrophage and T cell activation (McDevitt et al. 2004. Curr Hematol Rep 3:97-106). The appearance of BFU-E and CFU-E were quantified after culture with these cytokines, applied either alone or in combination. The addition of 10-100 ng/ml MIF, 1-20 ng/ml TNFα, or 10-100 U/ml IFNγ dose-dependently inhibited BFU-E and CFU-E colony formation (FIG. 1). The early stage, BFU-E showed greater inhibition than the late stage CFU-E colonies at each of the doses tested. The addition of sub-inhibitory concentrations of MIF together with TNFα and IFNγ resulted in a profound and synergistic inhibitory action.

MIF Suppresses Erythroid Differentiation in Progenitor Cell Lines.

Erythroid progenitors such as the Friend murine erythroleukemia (MEL) (Terada et al. 1977. Proc Natl Acad Sci USA 74:248-252) or the human K562 (Witt et al. 2000. Blood 95:2391-2396) cell line undergo cytodifferentiation in vitro, leading to the initiation of hemoglobin synthesis and to a more mature, erythroid phenotype. These model cell systems were examined because they replicate certain features of erythroid differentiation, and they are more amenable to biochemical analysis than methylcellulose cultures of primary progenitor cells.

Cultured MEL cells were induced to undergo cytodifferentiation in the presence of recombinant MIF and then analyzed for intracellular hemoglobin content by benzidine staining. At a test concentration of 200 ng/ml, which is an amount of MIF that may circulate during severe, systemic infection (Calandra et al. 2000. Nature Medicine 6:164-169 and Beishuizen et al. 2001. J Clin Endocrinol Metab 86:2811-2816), MIF decreased the cytodifferentiation response of MEL cells. The specificity of this effect was verified by the application of a neutralizing, anti-MIF mAb. Quantification of cellular hemoglobin by a sensitive chemical analysis also showed that lower amounts of MIF (20, 100 ng/ml) reduced hemoglobin content by 12% and 20% respectively.

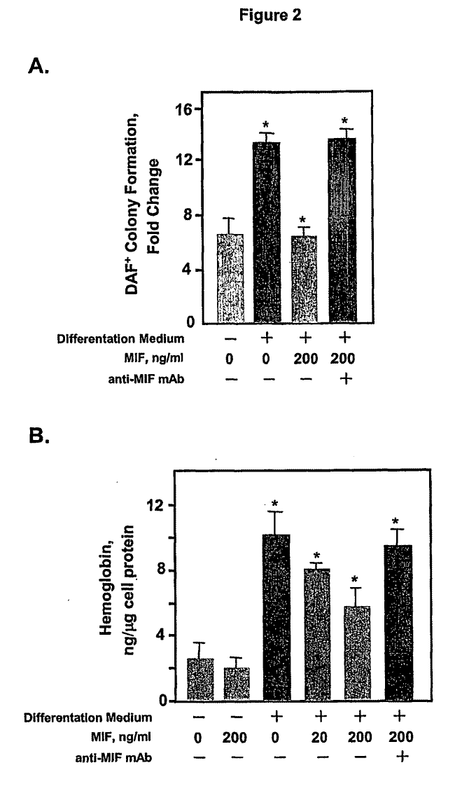

The effect of MIF stimulation on the human K562 progenitor cell line was then tested by co-culturing these cells under differentiation conditions in the presence of MIF. MIF inhibited the terminal erythropoietic differentiation of these cells, as measured by a sensitive diaminofluorene (DAF) stain (McGuckin et al. 2003. European Journal of Haematology 70:106-114). The impact of MIF on hemoglobin production in these cells also was verified by biochemical quantification of intracellular hemoglobin content (FIGS. 2A and 2B). Neither MIF nor anti-MIF were found to exert cytotoxic effects on erythroid progenitors, as assessed by Trypan blue exclusion.

MIF Modulates MAP Kinase Activation.

Having uncovered a prominent, inhibitory effect of MIF on the differentiation of K562 cells, MEL cells, and primary erythroid progenitors, the signaling mechanisms influenced by MIF stimulation was examined. Erythropoiesis requires the coordinate activation of several growth factor-dependent, signal transduction pathways (Arcasoy et al. 2005. Br J Haematol 130:121-129 and Klingmuller 1997 Eur J Biochem 249:637-647), and certain of these pathways may be faithfully represented in model, progenitor cell systems. For example, both primary erythroid and K562 progenitor cells exhibit differentiation-dependent modulation in the different subfamilies of the mitogen-activated protein (MAP) kinases (ERK-1/2, JNK-1/2, p38) (Terada et al. 1977. Proc Natl Acad Sci USA 74:248-252 and Witt et al. 2000. Blood 95:2391-2396).

MIF promotes pro-inflammatory functions in monocytes/macrophages and fibroblasts by activating the ERK-1/2 family of MAP kinases (Mitchell et al. 1999. J Biol Chem 274:18100-6). A distinguishing feature of MIF action is that it induces a sustained rather than a transient pattern of ERK-1/2 activation (Mitchell et al. 1999. J Biol Chem 274:18100-6), which is noteworthy because the kinetics of the ERK-1/2 activation influence significantly the differentiation pathway of different progenitor cell types (Howe et al. 1998. J Biol Chem 273:27268-27274). K562 progenitors were cultured in differentiation medium together with MIF, or MIF plus a neutralizing anti-MIF mAb, and the cell lysates were analyzed using phospho-specific antibodies directed against different MAP kinases. It was observed that the erythroid differentiation of K562 progenitors is associated with a time-dependent inhibition of ERK-1/2 and JNK-1/2 phosphorylation (beginning at 16 and at 96 hrs, respectively), and a complementary activation of the p38 phosphorylation (beginning at 16 hrs). MIF addition markedly affected these differentiation-associated, MAP kinase responses. In the case of the ERK-1/2 subfamily, phosphorylation was high at 16 hrs after MIF stimulation and, in a pattern that is consistent with other primary cell types (Mitchell et al. 1999. J. Biol. Chem. 274:18100-6), was sustained for a period of at least 96 hrs (16 hrs: p<0.01; 96 hrs, p<0.05). MIF addition also appeared to upregulate JNK-1/2 phosphorylation when compared to differentiation medium alone, however this effect was not pronounced as for ERK-1/2 and did not reach statistical significance (96 hrs, p<0.06). The differentiation-associated changes in p38 MAP kinase phosphorylation were reduced by MIF, however (16 hrs, p<0.01; 96 hrs, p<0.04). The phosphorylation patterns induced by MIF were normalized by anti-MIF mAb (96 hrs: ERK-1/2: p<0.04; p38: p<0.04, for anti-MIF mAb treatment vs. non-treatment), and the time course for these effects was consistent with the increase in hemoglobin synthesis observed after anti-MIF treatment. These data support the role of MIF in inhibiting erythroid differentiation by modulating MAP kinase activation.

The results presented above show that MIF is a direct and potent inhibitor of erythroid progenitor development.

MIF Mediates Anemia in Experimental Malaria Infection.

After intraperitoneal injection with a modest inoculum of P. chabaudi-parasitized red blood cells (10⁶ per mouse), the BALB/c mouse strain develops an acute parasitemia that peaks on approximately day 8 of infection (Stevenson et al. 2004. Nature Reviews Immunology 4:169-180). More than 50% of mice will succumb to infection by 3 weeks, and anemia contributes importantly to death since the administration of a blood transfusion late in infection can rescue up to 90% of the infected mice (Yap et al. 1994. Infect Immun 62:3761-3765).

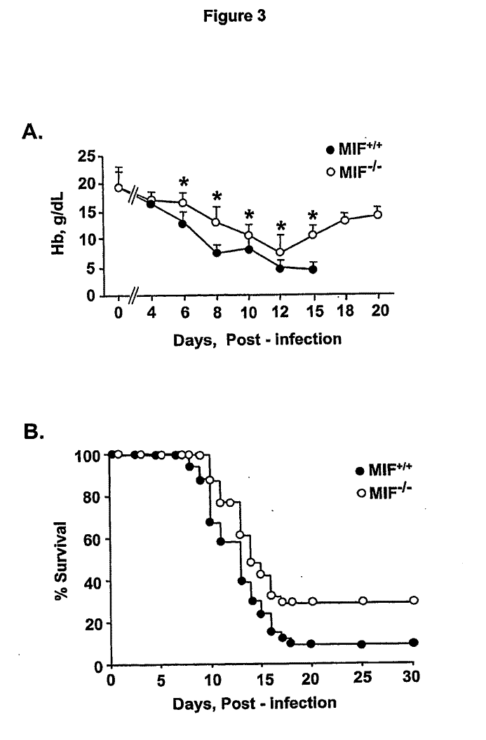

To determine the role of MIF in the anemic complications of acute malaria infection in vivo, a recently developed MIF-KO strain (Bozza 1999. J Exp Med 189:341-346) was backcrossed into the BALB/c genetic background for experimental infection with P. chabaudi. Before study, the hematopoietic competence of the MIF-KO strain was assessed by bone marrow histochemistry and enumeration of the different hematopoietic lineages. There were no significant differences between wild-type controls and MIF-KO mice with respect to the number of mature, peripheral blood cells, or in the numbers of CFU-E and BFU-E in bone marrow. Infection of wild-type or MIF-KO mice with P. chabaudi AS resulted in a significant parasitemia that peaked on post-infection day 8 at 47%±15%. Peripheral blood was sampled every two days and there was no significant difference in the mean level of parasitemia in the wild-type versus the MIF-deficient mice over the 4 week course of the study. Despite the similar levels of parasitemia however, the severity of anemia that developed in the two different experimental groups was quite different, especially as the infection progressed (FIG. 3A). Hemoglobin levels progressively declined in the wild-type mice, with the lowest levels recorded on post-infection day 15, after which time >90% of the animals in this group perished. By comparison, the MIF-KO mice experienced a less severe anemia than the wild-type mice, and this difference remained statistically significant after the first week. The onset of death for mice in the MIF-KO group was delayed by two days when compared to wild-type mice (P=0.013) (FIG. 3B). Moreover, while almost 30% of the MIF-KO mice survived to post-infection day 30, only 9% of wild-type mice survived this long (P<0.04).

To better assess the role of the host inflammatory response in the development of anemia and lethality from P. chabaudi infection, the production of MIF and the cytokines TNFα and IFNγ were assessed which serve as useful indicators of systemic macrophage and T cell activation, respectively. Plasma ME concentrations reached 40±20 ng/ml in wild-type mice at day 8. Serum TNFα and IFNγ levels showed no differences, however, when measured in the MIF-KO and the wild-type strain during the period critical for anemia and lethality. Taken together, these findings support an important role for MIF, independent of the contribution of TNFα and IFNγ, in the development of the malaria infection-related complications of anemia and death.

To further evaluate MIF's role in the development of anemia and erythroid suppression in this model of malaria infection, hematopoietic parameters were examined in these mice. Malaria infection produced a reticulocytosis in both the wild-type and the MIF-KO strains, however the corrected reticulocyte count was much greater in the setting of MIF deficiency (MIF^{+/+}: 8%±5% versus MIF^{-/-} 23%±10%; day 10 post-infection, P<0.01). The CFU-E and BFU-E in bone marrow were also measured, and colony formation was significantly greater in the P. chabaudi-infected, MIF-KO mice than in the corresponding, P. chabaudi-infected wild-type mice. Notably, the levels of bone marrow production of the cytokines TNFα and IFNγ were indistinguishable in the infected MIF-KO and wild-type strains, and the measured values mirrored closely the circulating levels of these mediators. The wild-type mice nevertheless showed a prominent induction of MIF protein in the bone marrow that increased over time during the period critical for anemia development. These data collectively support the role of MIF in erythroid suppression, and that MN-deficiency is associated with better compensation of erythropoiesis during experimental malarial infection.

In sum, these results show that MIF-KO mice infected with P. chabaudi showed less anemia and decreased mortality when compared to their wild-type, infected counterparts. The impact of MIF deficiency on either mortality or anemia in this experimental mouse model is consistent with MIF's influence on the component of anemia that is attributable to erythroid suppression. Enumeration of bone marrow cultures revealed significant increases in the CFU-E and BFU-E of the malaria-infected, MIF-KO mice when compared to the wild-type controls.

MIF Circulates in High Concentrations in Malaria Patients.

Recent investigations in human malaria have provided evidence for MIF production in the intervillous blood of pregnant women with placental infection (Chaisanvaneeyakorn et al. 2002. J Infect Dis 186:1371-1375 and Chaiyaroj et al. 2004. Acta Tropica 89:319-327). To determine whether clinically significant malaria is associated with an increase in MIF in the systemic circulation, a specific, sandwich ELISA was utilized to quantify MIF in patients residing in a P. falciparum-endemic region of Zambia. MIF was measured in 20 children presenting sequentially for clinical evaluation and diagnosed with malaria by peripheral blood smear. There was a significant increase in the plasma concentration of MIF in malaria-infected patients when compared to uninfected controls obtained from the same geographic region. The median plasma concentrations of MIF in the malarial patients was 11.8 ng/ml (0.9-49.5), which is comparable to the level reported previously in patients with bacterial sepsis (median: 12.2 ng/ml) (Calandra et al. 2000. Nature Medicine 6:164-169). These data support the conclusion that Plasmodium infection is a potent stimulus for the systemic expression of MIF in human subjects.

A Functional Promoter Polymorphism in Human MIF Influences the MIF Response to the Malarial Product, Hemozoin.

The evidence that MIF plays a role in malarial anemia prompted the examination of whether genetic polymorphisms in the human MIF gene influence the human host response to malaria infection. Functional, promoter polymorphisms in human MIF have been linked recently both to the incidence or the severity of several inflammatory diseases in different populations (Baugh et al. 2002. Genes Immun. 3:170-176; Donn et al. 2002. Arthritis & Rheum 46:2402-2409; Hizawa et al. 2004. American Journal of Respiratory & Critical Care Medicine 169:1014-8; and Mizue et al. 2005. Proc Natl Acad Sci USA 102:14410-14415). These genetic polymorphisms include a tetranucleotide sequence, CATT, that is repeated between 5-8 times in the MIF promoter (position −794).

Plasmodium-infected red cells stimulate MIF secretion by cultured macrophages (Martiney et al. 2000. Infection and Immunity 68:2259-2267). To study the MIF response of human cells, hemozoin was used as a stimulant. Hemozoin is a metabolite of Plasmodium hemoglobin degradation that accumulates within the reticuloendothelial system of the infected host (Slater, A. F. G. 1994. Malaria pigment. Exp Parasitol 74:362-365). Hemozoin induces cytokine release from monocytes/macrophages (Sherry et al. 1995. J Inflam 45:85-96 and Jaramillo et al. 2004. J Immunol 172:3101-3110) and it constitutes a Plasmodium-specific, “pathogen-associated molecular pattern” for innate immunity by interaction with the innate receptor, TLR9 (Coban et al. 2005. Journal of Experimental Medicine 201:19-25). Hemozoin, prepared synthetically from heme chloride (Sherry et al. 1995. J Inflam 45:85-96) and thus free of other Plasmodium-produced components, was found to induce MIF release from macrophages in a standardized culture system. Hemozoin was then added to human monocyte cultures of pre-determined MIF genotype. Cells bearing the low-expression, 5-CATT (homozygous) allele secreted lower levels of MIF than cells encoding the high-expression, 6-CATT (homozygous), or 6-CATT/7-CATT (heterozygous) alleles. This experimental data indicates that host MIF genotype influences MIF production in response to the malarial product, hemozoin.

Materials and Methods

Cytokines, Antibodies, and Reagents. Recombinant mouse or human MIF were prepared and the proteins purified essentially free of endotoxin (<1 pg endotoxin/μg protein) as described (Bernhagen et al. 1994. Biochemistry 33:14144-14155). The neutralizing anti-MIF mAb (IgG₁ isotype) and an isotypic, control antibody (5D4-11, ATCC, Manassas Va.) were purified by Mono-Q FPLC. Recombinant mouse TNFα (2.7×10⁵ U/μg) and IFN-γ (8.4×10³ U/μg) were from R&D Systems (Minneapolis, Minn.). Hemozoin was prepared synthetically from hemin chloride (Sigma) under endotoxin-free conditions (Sherry et al. 1995. J. Inflam. 45:85-96).

Cytokine Quantification. MIF was measured by sandwich ELISA employing human or mouse MIF-specific antibodies developed in the laboratory (Donnelly et al. 1997. Nature Medicine 3:320-323 and Calandra et al. 2000. Nature Medicine 6:164-169). The MIF content of bone marrow was determined by Western blotting and detection with rabbit polyclonal antibody (R102) (Donnelly et al. 1997. Nature Medicine 3:320-323). TNFα and IFNγ levels in sera and in supernatants of bone marrow lysates were measured by the Quantikine® M TNF-α or IFN-γ Immunoassay kit (R&D Systems, Inc., Minneapolis, Minn.).

Mice and Murine Cell Cultures. The MIF^{-/-} (MIF-KO) mice (Bozza 1999. J Exp Med 189:341-346) were bred onto the BALB/c genetic background by Charles River Laboratories (Wilmington, Mass.), and studied between 8-10 weeks of age. The mice were at generation N6. All mouse studies were performed in accordance with protocols approved by Institutional Animal Care and Use Committees.

Bone marrow precursors were harvested from the femora and tibia of mice in 3 ml of Iscove's MDM containing 2% FBS. The viability of marrow cells was determined to be >97% by Trypan blue exclusion staining. Bone marrow lysates were collected by flushing two femurs and two tibias per mouse with 1 ml of lysis buffer (150 mM NaCl, 50 mM Tris, pH 7.5, 1% NP-40, 0.5% deoxycholic acid, 0.1% SDS, and 2 mM EDTA) using a 25 G5/8 gauge needle. The bone marrow plug was homogenized, the cellular debris pelleted, and the lysate supernatant concentrated using an Amicon Centricon 10 membrane (Amico, Beverly, Mass.). Mouse macrophages were prepared from the adherent cultures of thioglycollate-elicited peritoneal macrophages, as described previously (Calandra et al. 1994. J. Exp. Med. 179:1895-902).

BFU-E and CFU-E Progenitor Cell Assays. The progenitor cell (colony) assays were performed according to the standardized methods described previously (Martiney et al. 2000. Infection and Immunity 68:2259-2267) and protocols from StemCell Technologies (Vancouver; BC, Canada). Briefly, washed murine bone marrow cells were plated in sterile 35-mm dishes containing a methylcellulose-based medium and growth factors. The total number of bone marrow cells plated in duplicate culture dishes were as follows: 2×10⁵ for CFU-E and 5×10⁵ for BFU-E. MethoCult™ M3334 was utilized for the CFU-E and BFU-E colony assays. The media for CFU-E and BFU-E was volume adjusted by adding 1 part Isocove's MDM to 9 parts MethoCult™ M3334 to give final concentrations of the following components: 15% fetal bovine serum, 1% BSA, 200 mg/ml transferrin, 10 mg/ml insulin, 1% methylcellulose, 10⁻⁴ M 2-mercaptoethanol, 2 mM L-glutamine and 3 units/ml erythropoietin. Each assay of different bone marrow progenitor cells was performed independently 3-6 times.

Erythroid Progenitor Cell Lines. The Friend murine erythroleukemia (MEL) cell line was cultured in Dulbecco's modified Eagle's medium supplemented with 10% FBS. Differentiation was induced with dimethyl sulfoxide differentiation medium for 4 days as described (Terada et al. 1977. Proc. Natl. Acad. Sci. USA 74:248-252). Hemoglobin synthesis was assessed by cyto-centrifugation onto glass slides followed by staining with benzidine (Worthington), and by the hemoglobin kit from Sigma.

The human K562 progenitor cell line (ATCC, CCL-243) was cultured in RPMI/10% FBS supplemented with penicillin and streptomycin. For differentiation, sodium butyrate (AldRick, Milwaukee, Wis.) was added in the medium (Witt et al. 2000. Blood 95:2391-2396). Terminal erythropoietic differentiation was measured as described by McGukin et al. (McGuckin et al. 2003. European Journal of Haematology 70:106-114) and positive cells enumerated after staining with 2,7-diaminofluorene (Sigma, St. Louis, Mo.). To quantify hemoglobin synthesis, the cells were centrifuged and washed in PBS, and the cell pellet resuspended in lysis buffer (100 mM potassium phosphate pH 7.8, 0.2% Triton X-100) before disruption by aspiration through a 21-gauge needle. After microcentrifugation, the supernatant was collected and the hemoglobin concentration was determined using the hemoglobin kit from Sigma. Cell viability was assessed by trypan blue dye exclusion and found to be equivalent (85-90% viability) between all samples studied.

Western Blot Analysis. Immunoblotting with antibodies directed against phospho-ERK1/2, total ERK1/2, phospho-JNK1/2, total JNK1/2, phospho-p38, and total p38 was performed according to the manufacturer's instructions (New England Biolabs, Beverly, Mass.). The secondary antibody was an anti-rabbit IgG conjugated to horseradish peroxidase and detection was by chemiluminescence according to the manufacture's instructions (Amersham Pharmacia Biotech, Piscataway, N.J.). For quantitation of signal intensity of phospho-specific kinase results, western blots were scanned using a UMAX, PowerLook IIII scanner and the images were analyzed using UN-SCAN-IT gel Automated Digitizing system software version 5.1 (Silk Scientific Corporation, USA). The ratio between phosphorylated to the total MAP kinase was determined and expressed as a fold-change for each lane (mean±S.E.M for three blots from independent cell cultures). The p values were calculated for each of the time course comparisons (4, 16, 96 hrs) of three different experiments (Student T-test, two-tailed).

Mouse Malaria Studies. To maintain uniformity of infections, P. chabaudi AS was maintained by serial passage in A/J mice and no more than ten passages were allowed before a fresh inoculum was prepared from frozen stocks. For simple passages, infection was initiated with a dose of 10⁶-10⁷ parasitized erythrocytes. For clinical infections, mice were inoculated intraperitoneally with 10⁶ parasitized red blood cells. The course of experimental infection in mice was monitored every other day by examining DiffQuik (Baxter Scientific Products, West Chester, Pa.)-stained thin smears from blood. Parasitemias were determined by counting a minimum of 200 erythrocytes per blood sample. Hemoglobin was determined by using Drabkin's procedure (Sigma Diagnostics, St Louis, Mo.) on 1 μl of tail vein blood prepared on every second day, post-infection, for 3 weeks. Mice were observed daily for at least 30 days, and mortality was recorded. At pre-determined times post-infection, five animals per group were euthanized by CO₂, and the blood was collected by cardiac puncture.

Human Specimens and Cell Culture. Blood samples from 20 malaria patients and 20 uninfected, healthy controls were obtained from the Macha Mission Hospital in Choma, Zambia, where P. falciparum is endemic. The blood samples were obtained in accordance with research protocols approved by the institutional review boards of the University of Zambia and Yale University. The subjects had acute onset malaria and were diagnosed from clinical symptoms (fever, splenomegaly, headache, malaise) and a positive thick-blood film for parasites. The subjects (12 M, 8 F) were between 5-52 months of age (mean=25.5 months) and had a mean Hg of 5.9 gm %. Co-infection by organisms likely to contribute to a systemic inflammatory response was excluded by clinical evaluation and laboratory testing. Numerical values obtained by MIF-specific sandwich ELISA were confirmed, semi-quantitatively, by Western blotting.

Human mononuclear cells were prepared from the adherent leukocyte fraction of volunteers and genotyped for the (−794) CATT tetranucleotide repeat and for a (−173) G/C SNP (Baugh et al. 2002. Genes Immun. 3:170-176). Two homozygous and one heterozygous MIF promoter genotypes were studied: 5-CATT/5-CATT; 6-CATT/6-CATT; and 6-CATT/7-CATTC. The mononuclear cells were cultured in 24 well plates (10⁴ cells/well) and stimulated with hemozoin (42 nM) for 48 hrs. Cells from 3 individuals were studied within each genotyped group, and the results were repeated twice

Statistics. Data are presented as means±standard deviations (SD). The significance level for all statistical tests was set at α=0.05. One-way analysis of variance (ANOVA) was carried out to determine whether there were significant differences in the effects of MIF, anti-MIF, TNFα and IFNγ, at various doses with respect to colony formation of bone marrow progenitor cells. Upon finding significant differences, Bonferroni-adjusted pairwise (post-hoc) comparisons were carried out to determine which mediator-dose combination differed from one another. Comparisons between cytokine levels, colony formation and cytokine production between the MIF^{+/+} and MIF^{-/-} mice, and between normal and malaria-infected mice, were carried out using the Student's t-test (two-tailed). For mouse survival, the median survival time was compared by the Mann-Whitney, and the overall survival by the χ2 tests (Zar J H. 1984. Biostatistical Analysis. Prentice-Hall, Englewood Cliffs, N.J.).

### Example 2

### Thin-Film Biosensor Chips for Genotyping MIf Polymorphisms and its Use to Genotype Malaria Patients

This example summarizes the development and implementation of a thin-film optical biosensor chip for genotyping both tetranucleotide CATT repeats and SNPs in a single analysis that greatly facilitates the examination of small quantities of DNA which are usually obtained in the field. These chips are based on an allele-discriminating oligonucleotide array that provides a direct visual readout of the different MIF promoter polymorphisms.

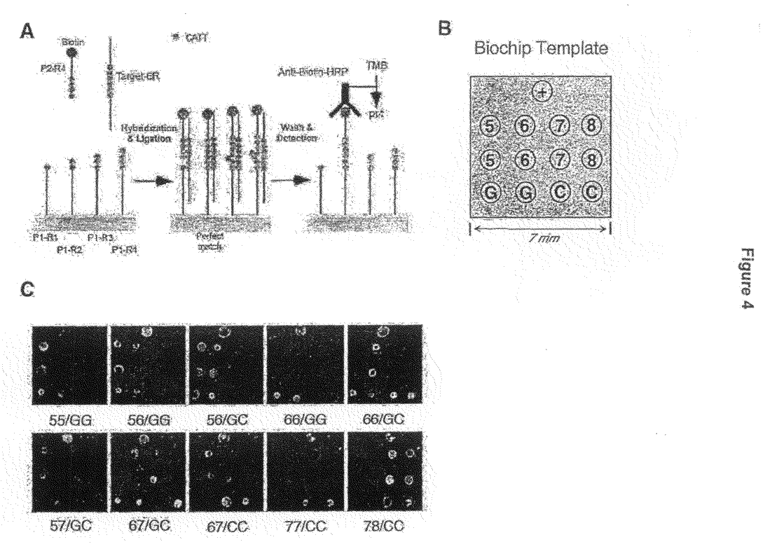

The MIF CATT repeat polymorphism and the −173 G/C SNP detection were performed by modifying solid-phase techniques described previously (Thong et al. (2003) Proc. Natl. Acad. Sci. USA, 100, 11559-11564). Prior ligation-based assays have been found to be efficient only for discriminating polymorphic alleles of short microsatellite repeats <30 bases (Zhong et al. (2003) Proc. Natl. Acad. Sci. USA, 100, 11559-11564 and Zirvi et al. (1999) Nucleic Acids Res., 27, e41). Ligation of longer repeats lacks specificity because of the looping out of repeat units in the probe/target hybrids. The loopout often occurs in an area outside of the ligase footprint that can span 26-30 bases (Zirvi et al. (1999) Nucleic Acids Res., 27, e41). To avoid the formation of loopouts in the MIF CATT repeat sequence, the repeat was divided into the capture probes ‘P1’, with CATT 1, 2, 3 or 4, and ‘P2’, with CATT4 (FIG. 4A), thereby ensuring that the ligase footprint covers 6-7-CATT repeat units. Four capture probes with 1, 2, 3 and 4 CATT repeat units, respectively, together with 30 bases of the upstream adjacent unique MIF sequence (see detailed sequences in Table 2) were covalently arrayed by the reaction between an aldehyde group at their 5′-termini and the hydrazine group on the chip surface. A detection probe, P2, with 4 copies of the CATT unit and 20 bases of the downstream adjacent and unique MIF sequence carried a biotin at the 3′ end for detection and a phosphate at its 5′ end for ligation. When target DNA with a variable number of the CATT unit (from 5 to 8), is hybridized with the P1 and P2 probes on the chip surface in the presence of a thermostable DNA ligase, the enzyme selects only perfectly matched P1+P2/target for ligation. This reaction resulted in the immobilization of the biotinylated P2 probe at the matched P1 probe position on the chip surface. After a stringent wash to remove all non-ligated molecules, a biotin-associated detection procedure was applied to generate a color that was visualized by the unaided human eye. The biochips can also be printed by high-throughput ‘spotting’ of the requisite capture probes onto hydrazine-functionalized thin-film biosensor chips. The entire methodology is low in cost and once printed, the chips are robust and require no instrumentation for analysis.

The P1 capture probes were manually spotted on hydrazine-functionalized, thin-film biosensor chips (Thermo Electron, Louisville, Colo.) using 0.3 μl of 1 μM P1 in 0.1 M phosphate buffer, pH 7.8 and 10% glycerol. After incubation for 2 h at room temperature in a humid chamber, the chips were washed with 0.1% SDS at 60° C. for 30 min, rinsed with water and dried. A ligation mixture containing 20 mM Tris-HCl, pH 8.3, 25 mM KCl, 10 mM MgCl₂, 0.5 mM NAD, 0.01% Triton X-100, 5 mg/ml acid-treated casein, 10% formamide and 0.04 U/μl mutant Ampligase (Lys294Arg of Thermus thermophilus ligase) was applied to each chip and prewarmed to 60° C. The synthetic targets or MIF PCR amplicons from patient samples at a concentration of 100 fmol in 10 μl water were denatured at 95° C. for 3 min in the presence of 100 fmol of the P2-biotin probe. After denaturation, 10 μl of this solution was immediately added to the pre-incubated ligation mixture and incubated at 60° C. for 20 min. A stringent wash with 0.01 M NaOH at 60° C. was applied three times, and this was followed by briefly rinsing three times with 0.1× standard saline citrate (SSC) at room temperature. The chips were then incubated at room temperature for 10 min with 100 μl of an anti-biotin-IgG-horseradish peroxidase conjugate (Jackson ImmunoResearch Lab; 1:1000 dilution from a 1 mg/ml stock in a buffer containing 5×SSC and 5 mg/ml acid-treated casein). After briefly washing three times with 0.1×SSC, 100 μl of a tetramethyl benzidine formulation from BioFX Lab was added to the chips and incubated for 5 min at room temperature, then rinsed with ddH₂O and air dried. The CATT and SNP polymorphisms were scored visually and the results were recorded by imaging under a dissection microscope fitted with a digital camera.

Using single-strand synthetic targets with tetranucleotide repeats of 5, 6, 7 or 8 AATG, the detection specificity was detected for the different MIF CATT repeat sequences. A modified ligation condition with 10% formamide in the standard ligation mixture (Zhong et al. (2003) Proc. Natl. Acad. Sci. USA, 100, 11559-11564) gave unambiguous discrimination of the CATT repeat copy number. The allele-specific ligation strategy was also used for the simultaneous detection of the MIF SNP G/C −173.

To test for the accuracy of genotyping the CATT repeat polymorphism and the −173 SNP simultaneously by the biosensor chip, an initial 30 genomic DNA samples that had been previously studied for MIF genotype were analyzed. The CATT repeat polymorphism is detected by capillary electrophoresis of PCR products amplified to span the tetranucleotide region, and the −173 G/C SNP is determined by the ddNTP primer extension method and capillary electrophoresis, or by pyrosequencing (Baugh et al. (2002) Genes Immun., 3, 170-176). The typings of the CATT repeat and −173 SNP obtained by using the biosensor methodology were compared initially in a blinded fashion. Representative images are shown in FIG. 4C and the optical biosensor genotypes are listed in Table 3. There was complete concordance of the −173 G/C SNP genotypes between the two methods. One discrepancy was observed for a previously analyzed DNA sample bearing the 8-CATT repeat. The initial chromatographic analysis for sample 1418 showed that one PCR product peak eluted in an intermediate position for that expected is either the 7-CATT or the 8-CATT repeat. The peak was scored as ‘8-CATT’ by the capillary electrophoresis (5/8), but 7-CATT by the biosensor chip (5/7). Subsequent reanalysis of this DNA sample by capillary electrophoresis verified the presence of 7-CATT and the scoring of this sample as ‘5/7’. The concordance with typing data for the −173 G/C SNP and the ability to identify errors in the CATT repeat obtained by capillary electrophoresis indicate that genotyping by optical biosensor chips is highly accurate.

In cases where limited amounts of genomic DNA are available, such as with DNA obtained from small amounts of blood, a pre-amplification step may be necessary. One method for DNA amplification is multiple displacement amplification (MDA). To evaluate concordance between MDA products and unamplified genomic DNA for the ligation-based optical biosensor methodology, the 30 samples genotyped in the as described above were re-evaluated after MDA. Complete fidelity was observed between the unamplified genomic DNA and the MDA products for both the MIF CATT polymorphism and the −173 G/C SNP genotype.

TABLE 2
Oligonucleotide sequences of capture probe P1,
detection probe P2, synthetic targets and PCR
primers

CATT repeat

| | |
|---|---|
| Capture | P1-CATT-1 |
| probe P1 | 5′-CHO-AAAAAAAAAACTGCTATGTCATGGCTTATCTT |
| | CTTTCACCCATT |
| | (SEQ ID NO: 5) |
| | P1-CATT-2 |
| | 5′-CHO-AAAAAAAAAACTGCTATGTCATGGCTTATCTT |
| | CTTTCACCCATTCATT |
| | (SEQ ID NO: 6) |
| | P1-CATT-3 |
| | 5′-CHO-AAAAAAAAAACTGCTATGTCATGGCTTATCTT |
| | CTTTCACCCATTCATTCATT |
| | (SEQ ID NO: 7) |
| | P1-CATT-4 |
| | 5′-CHO-AAAAAAAAAACTGCTATGTCATGGCTTATCTT |
| | CTTTCACCCATTCATTCATTCATT |
| | (SEQ ID NO: 8) |
| Detection | P2 biotin |
| probe P2 | 5′-phosphate-CATTCATTCATTCATTCAGCAGTATT |
| | AGTCAATGTC-biotin-3′ |
| | (SEQ ID NO: 9) |
| Synthetic | Target AATG-5 |
| target | GACATTGACTAATACTGCTGAATGAATGAATGAATGAAT |
| | GGGTGAAAGAAGATAAGCCATGACATAGCAG |
| | (SEQ ID NO: 10) |
| | Target AATG-6 |
| | GACATTGACTAATACTGCTGAATGAATGAATGAATGAAT |
| | GAATGGGTGAAAGAAGATAAGCCATGACATAGCAG |
| | (SEQ ID NO: 11) |
| | Target AATG-7 |
| | GACATTGACTAATACTGCTGAATGAATGAATGAATGAAT |
| | GAATGAATGGGTGAAAGAAGATAAGCCATGACATAGCAG |
| | (SEQ ID NO: 12) |
| | Target AATG-8 |
| | GACATTGACTAATACTGCTGAATGAATGAATGAATGAAT |
| | GAATGAATGAATGGGTGAAAGAAGATAAGCCATGACATA |
| | GCAG |
| | (SEQ ID NO: 13) |
| PCR | Forward primer: |
| primer and | CTATGTCATGGCTTATCTTC |
| product | (SEQ ID NO: 14) |
| | Reverse primer: |
| | TCCACTAATGGTAAACTCGG |
| | (SEQ ID NO: 15) |
| | PCR products: |
| | 119, 123, 127 or 131 |
| | CTATGTCATGGCTTATCTTCTTTCACC(CATT)5-8CAG |
| | CAGTATTAGTCAATGTCTCTTGATATGCCTGGCACCTGC |
| | TAGATGGTCCCCGAGTTTACCATTAGTGGA |
| | (SEQ ID NO: 16) |

SNP173-G/C

| | |
|---|---|
| Capture | P1-G |
| probe P1 | 5′-CHO-AAAAAAAAAACCGGAACAGGCCGATTTCTAGC |
| | CGCCAAGTGGAGAACAGG |
| | (SEQ ID NO: 17) |
| | P1-C |
| | 5′CHO-AAAAAAAAAACCGGAACAGGCCGATTTCTAGCC |
| | GCCAAGTGGAGAACAGC |
| | (SEQ ID NO: 18) |
| Detection | P2 biotin |
| probe P2 | 5′-phospbate-TTGGAGCGGTGCGCCGGGCTTA- |
| | biotin-3′ |
| | (SEQ ID NO: 19) |
| Synthetic | MIF-173 Target G |
| target | AGCCCGGCGCACCGCTCCAACCTGTTCTCCACTTGGCGG |
| | CTAGAAATCGGCCTGTTCCGG |
| | (SEQ ID NO: 20) |
| | MIF-173 Target C |
| | AGCCCGGCGCACCGCTCCAAGCTGTTCTCCACTTGGCGG |
| | CTAGAAATCGGCCTGTTCCGG |
| | (SEQ ID NO: 21) |
| PCR | MIF-173 Forward |
| primer and | ACTAAGAAAGACCCGAGGCG |
| product | (SEQ ID NO: 22) |
| | MIF-173 Reverse |
| | GCAGGACCCTGGGCGACT |
| | (SEQ ID NO: 23) |
| | PCR product 129 bp |
| | ACTAAGAAAGACCCGAGGCGAGGCCGGAACAGGCCGATT |
| | TCTAGCCGCCAAGTGGAGAACAGGTTGGAGCGGTGCGCC |
| | GGGCTTAGCGGCGGTTGCTGGAGGAACGGGCGGAGTCGC |
| | CCAGGGTCCTGC |
| | (SEQ ID NO: 24) |

The underlined sequences designate the unique repeated sequences (5-8 repeats) present in the synthetic oligonucleotide target The bolded residue designates the −173 G/C SNP.

TABLE 3
Comparison of MIF genotypes of the CATT tetranucleotide repeat
and the −173 G/C SNP in a selection of human DNA samples by
optical biosensor chip and capillary electrophoresis

| | CATT repeat | | −173 G/C SNP | |
|---|---|---|---|---|
| | | Optical | | Optical |
| Sample | Capillary | biosensor | Capillary | biosensor |
| ID | electrophoresis | chip | electrophoresis | chip |
| 0009 | 5/6 | 5/6 | G/G | G/G |
| 0010 | 6/6 | 6/6 | G/G | G/G |
| 0013 | 6/6 | 6/6 | G/G | G/G |
| 0014 | 5/5 | 5/5 | G/G | G/G |
| 0015 | 6/7 | 6/7 | C/G | C/G |
| 0132 | 5/5 | 5/5 | G/G | G/G |
| 0134 | 5/5 | 5/5 | G/G | G/G |
| 0209 | 5/6 | 5/6 | G/G | G/G |
| 0306 | 5/6 | 5/6 | G/G | G/G |
| 0422 | 5/6 | 5/6 | G/G | G/G |
| 0425 | 5/7 | 5/7 | G/C | G/C |
| 0428 | 6/6 | 6/6 | G/G | G/G |
| 0429 | 5/7 | 5/7 | G/C | G/C |
| 0431 | 5/6 | 5/6 | G/G | G/G |
| 0506 | 6/6 | 6/6 | G/G | G/G |
| 0507 | 6/7 | 6/7 | C/C | C/C |
| 0602 | 7/7 | 7/7 | C/C | C/C |
| 0603 | 5/6 | 5/6 | G/C | G/C |
| 0807 | 6/7 | 6/7 | G/C | G/C |
| 1003 | 6/6 | 6/6 | G/G | G/G |
| 1203 | 6/6 | 6/6 | G/G | G/G |
| 1206 | 6/6 | 6/6 | G/C | G/C |
| 1207 | 6/6 | 6/6 | G/C | G/C |
| 1308 | 5/6 | 5/6 | G/G | G/G |
| 1418^{a} | 5/8^{a} | 5/7^{a} | G/C | G/C |
| 1603 | 6/6 | 6/6 | G/G | G/G |
| 1606 | 5/6 | 5/6 | G/G | G/G |
| 0036 | 7/7 | 7/7 | C/C | C/C |
| 0914 | 7/8 | 7/8 | C/C | C/C |
| 1214 | 6/7 | 6/7 | C/C | C/C |

Different samples were first typed by biosensor chip methodology, and then verified by capillary electrophoresis and pyrosequencing.
^{a}A discrepancy reading between optical biosensor chip and capillary electrophoresis.

Genotyping of MIF −173 G/C and −794 GATT Repeat Polymorphisms in Rural Populations

Forty samples from patients in Zambia with P. falciparum malaria were collected and dried onto filter papers, stored at ambient temperature and the genomic DNA extracted with the QIAamp DNA Blood Mini kit (Qiagen). DNA yields were reliably 10-20 ng per sample. Ten nanograms of the isolated, genomic DNA was used for whole genome amplification by MDA in a standard 50 μl reaction, resulting in 15-20 μg of the MDA product per reaction. Targets for the MIF polymorphic genotyping were prepared by PCR of 100 ng of MDA-amplified products. The genotyping was performed on optical biosensor chips, and the genotypes and allele frequencies were compared with that of a previously reported Caucasian control group (Table 4). The initial data indicated a possible difference in the allelic distribution of MIF genotypes in different populations. In particular among the Zambian samples, there was a relative increase in the proportion of the low-expression 5-CATT allele, and a corresponding decrease in the proportion of the high-expression 6-CATT and 7-CATT alleles when compared with the recent studies in Caucasian populations (Baugh et al. (2002) Genes Immun., 3, 170-176 and Barton (2003) Genes Immun., 4, 487-491). Furthermore, the SNP analysis revealed a relative decrease in the −173G and a corresponding increase in the −173C in the Zambian samples when compared with previously studied Caucasian groups. Small representative sets of DNA samples obtained from four different North African tribes and one African-American group were then analyzed. The allelic distribution, when summed over the different groups, appeared closer in distribution to the Zambian group than that described previously in Caucasian groups (included in Table 4) (Baugh et al. (2002) Genes Immun., 3, 170-176 and Barton (2003) Genes Immun., 4, 487-491).

TABLE 4
Allele and genotype frequencies of the MIF polymorphisms in Caucasian
and African populations

| | | | | | | | Allele frequency | | |
|---|---|---|---|---|---|---|---|---|---|
| MIF polymorphism | Genotype, frequency no. (%) | | | | | | (%) | | |
| CATT(5-8) | 5/5 | 5/6 | 5/7 | 6/6 | 6/7 | 7/7 | 5 | 6 | 7 |
| Caucasian^{a} (n = | 8 | 61 | 10 | 53 | 25 | 0 | 27.3 | 60.3 | 11.0 |
| 159) | (5.0) | (38.3) | (6.3) | (33.3) | (15.7) | (0) | | | |
| Zambia malaria (n = | 14 | 14 | 3 | 5 | 1 | 3 | 56.3 | 31.2 | 12.5 |
| 40) | (35.0) | (35.0) | (7.5) | (12.5) | (2.5) | (7.5) | | | |
| African control | 5 | 19 | 4 | 5 | 7 | 0 | 41.3 | 45.0 | 13.7 |
| groups | (12.5) | (47.5) | (10.0) | (12.5) | (17.5) | (0) | | | |
| (n = 40) | | | | | | | | | |
| −173 SNP G/C | G/G | G/C | C/C | | | | G | C | |
| Caucasian^{b} (n = 88) | 67 | 21 | 0 | | | | 88.1 | 11.9 | |
| | (76.1) | (23.9) | (0) | | | | | | |
| Zambia malaria (n = | 12 | 12 | 16 | | | | 45.0 | 55.0 | |
| 40) | (30.0) | (30.0) | (40.0) | | | | | | |
| African control | 6 | 19 | 15 | | | | 38.7 | 61.3 | |
| groups | (15.0) | (47.5) | (37.5) | | | | | | |
| (n = 40) | | | | | | | | | |

^{a}Data cited from Baugh et al. (2002) Genes Immun., 3: 170-176.
^{b}Data cited from Donn et al. 2002. Arthritis & Rheum 46: 2402-2409.

Logistic regression analysis revealed that patients with the 5/X genotype (where X=6-, 7- or 8-CATT allele) were significantly less likely to have parasitemia >10 000 than those with an X/X genotype (P=0.04) (Table 5). The association of low-expression MIf alleles with likelihood of developing parasitemia can provide prognostic information and influence treatment options.

TABLE 5
Association between parasitemia level on admission and MIF
genotype for children evaluated at the Macha Mission Hospital
in Zambia

MIF genotype
(n, %)

| Parasitemia | 5/X | X/X | P-value |
|---|---|---|---|
| ≦10 000 | 27 (84.4) | 5 (71.4) | 0.04 |
| >10 000 | 5 (15.6) | 2 (28.6) | |

5 = low-expression 5-CATT MIF allele;
X = higher expression 6-, 7- or 8-CATT MIF alleles.

A set of 100 control (disease-free) specimens obtained from Korea were also examined. This analysis showed an allelic distribution that was intermediate in 5-CATT (62%, 5/5 or 5/X) and non-5-CATT (38%, X/X) frequency when compared with the African or previously analyzed Caucasian groups (Baugh et al. (2002) Genes Immun., 3, 170-176 and Barton (2003) Genes Immun., 4, 487-491), and is in agreement with the proportions reported in a recent study of a Japanese population (Hizawa (2004) Am. J. Respir. Crit. Care Med., 169, 1014-1018).

The robust optical biosensor methodology described herein has high fidelity when compared with the currently used methods and instrumention, and it can be readily combined with the MDA technique to analyze minute quantities of DNA extracted from dried, whole blood specimens. This method will greatly facilitate the genotyping and population studies in different field settings, and it makes possible the rapid translation of genotyping information in the clinic into medical intervention.

Materials and Methods

Patient Samples

DNA specimens from normal controls of known MIF genotype were used to develop the biosensor methodology. Validation was performed on a random selection of DNA specimens culled from ongoing studies of MIF genotype, and included Caucasians, African-Americans, North East Asians (Koreans) and Africans (Zambia and North Africa). The African sub-group specimens were collected either by K. K. Kidd and J. R. Kidd, or by one of their collaborators as part of their long-term studies on human genetic diversity. The malaria-infected samples were obtained as part of investigations at the Macha Mission Hospital in Choma, Zambia, which is holoendemic for Plasmodium falciparum malaria. These specimens consisted of blood samples that had been spotted on filter paper, dried and stored for several months. Parasitemia was assessed on hospital admission by enumeration of infected red cells in thick blood smears. All samples were obtained and utilized in accordance with institutional and local IRB protocols. The SAS statistical software package was used to perform analysis and logistic regression to determine the relationship between parasitemia and MIF genotype.

DNA Extraction

Genomic DNA was isolated from patient blood or from the blood spots dried on filter papers using the QIAamp DNA Blood Mini kit (Qiagen, Valencia, Calif.) and the recommended protocols provided by the manufacturer.

DNA Amplification by Multiple Displacement Amplification

DNA extracted from blood spots dried on filter papers was amplified by multiple displacement amplification (MDA) (Dean et al. (2002) Proc. Natl. Acad. Sci. USA, 99, 5261-5266). The MDA reactions were performed overnight at 31° C. in 50 μl of reaction solution containing 37.5 mM Tris-HCl (pH 7.5), 50 mM KCl, 10 mM MgCl₂, 5 mM (NH₄)₂SO₄, 1 mM dNTP, 0.05 mM thiophosphate-modified random hexamer (5′-NNNN^{s}N^{s}N), 1-5 ng genomic DNA, 0.2 U yeast pyrophosphatase and 1 U Phi29 polymerase. The products of the MDA reaction were resolved on a 1% agarose gel stained with ethidium bromide. The yield of amplified genomic DNA was between 15 and 20 μg in a 50 μl reaction solution.

MIF DNA Fragment Amplification by PCR

Oligonucleotide primer sets were designed to amplify regions within the MIF promoter that corresponded to DNA product sizes of 119, 123, 127 and 131 by for the 5-, 6-, 7- and 8-CATT repeat polymorphisms, respectively (Table 2). An additional set of primers was used for the −173 G/C SNP and produced a PCR product size of 129 bp. The PCRs were carried out in a 50 μl solution containing 1×PCR buffer (AmpliTaq Gold; Applied Biosystem), 2.5 mM MgCl₂, 0.2 mM dNTP, 10 pmol of each forward and reverse primers, 100 ng of genomic DNA or MDA-amplified genomic DNA and 1 U AmpliTaq Gold DNA polymerase. For the CATT repeat, the PCR program was as follows: 95° C. for 10 min, followed by 40 cycles at 94° C. for 30 s, 56° C. for 30 s, 72° C. for 1 min and final extension at 72° C. for 10 min, and for the −173 SNP, the PCR program was 95° C. for 10 min, followed by 40 cycles of 94° C. for 30 s, 62° C. for 30 s, 72° C. for 1 min and final extension at 72° C. for 10 min.

Oligonucleotide Probe Synthesis

The oligonucleotide probes were designed based on the general approach described previously (Zhong et al. (2003) Proc. Natl. Acad. Sci. USA, 100, 11559-11564), and with the detailed sequence information provided in Table 2. For the CATT repeat, the 4P1 capture probes have 5′-aldehyde groups, 10 deoxyadenosine residues at their 5′ ends and 30 bases of upstream sequence, followed by a different number (from 1 to 4) of the CATT repeat, respectively. The P2 detection probe contains 4 copies of CATT and 20 bases of downstream sequence, with a biotin at its 3′ end for detection and a phosphate group at its 5′ end for ligation. The P1 and P2 probes were synthesized by the Yale Keck Biotechnology Facility at 40/50 nmol scale and were used without post-synthesis purification. Four complementary, single-stranded artificial targets with 5-8 copies of the AATG repeat flanked by 30 bases of the MIF promoter sequences both upstream and downstream of the repeat motif also were synthesized.

### Example 3

### MIF Polymorphisms are Associated with Mortality after Cap

It was examined whether high-expressing MIF alleles were associated with clinical outcome in patients with CAP. Patients were genotyped for their MIF haplotype and monitored for the development of severe sepsis and 90-day mortality. To assess susceptibility to CAP, a case-control design was used, comparing cases (subjects who presented to Emergency Departments [EDs] with CAP) to healthy controls. To assess risk of developing severe sepsis and death, cohort design was used, comparing the hospitalized CAP cases who did and did not develop the outcomes of interest.

### Methods

Study Design

CAP cases were recruited as part of the Genetic and Inflammatory Markers of Sepsis (GenIMS) study, a large, multicenter study of subjects presenting to the EDs of 28 teaching and non-teaching hospitals in 4 regions (Western Pennsylvania, Connecticut, Michigan, and Tennessee) between November 2001 and November 2003. Eligible subjects were >18 years and had a clinical and radiologic diagnosis of pneumonia. Exclusion criteria were: transfer from another hospital; discharge from a hospital within the prior 10 days; episode of pneumonia within the past 30 days; chronic mechanical ventilation dependency, cystic fibrosis; active pulmonary tuberculosis; positive HIV antibody titer; alcoholism with evidence of end-organ damage; admission for palliative care; prior enrollment in the study; incarceration, and; pregnancy. Informed consent was obtained from the patient or a proxy. The Institutional Review Boards of the University of Pittsburgh and all participating sites approved the study. All human participants gave written informed consent.

Of the 2320 subjects enrolled in the GenIMS cohort, 137 subjects were excluded because their treating physician subsequently ruled out CAP. Of the 2183 remaining subjects, we restricted our analysis to 1738 non-Hispanic white subjects. Race was based on participant self-report. We limited our analysis to non-Hispanic whites because the frequency of MIF polymorphisms differs across race (Zhong et al. Nucleic Acids Res 2005; 33:e121) we had insufficient sample size to explore associations in other racial groups, and previously reported associations for these genotypes were in whites. Healthy controls comprised 639 non-Hispanic white volunteers who were recruited from the Northeast and Midwest regions of United States and who reported no chronic disease.

Clinical and Outcome Variables

Baseline and sequential clinical information was gathered by structured subject or proxy interviews, bedside assessment by study nurses, and from medical records. Pre-hospitalization comorbid conditions were ascertained using the Charlson comorbidity index (Charlson et al. J. Chronic Dis. 1987; 40:373-83). Severity of illness was assessed using APACHE III and the Pneumonia Severity Index (Fine et al. N. Engl. J. Med 1997; 336:243-50 and Knaus et al. Chest 1991; 100:1619-36). The outcome variables for the cohort design were development of severe sepsis and 90-day mortality. Severe sepsis was defined as pneumonia plus acute organ dysfunction following the International Consensus Criteria (Levy et al. 2001 Crit. Care Med. 2003; 31:1250-56). Acute organ dysfunction was defined as a new Sequential Organ Failure Assessment (SOFA) score of ≧3 in any of six organ systems, following the Sepsis Occurrence in the Acutely ill Patient (SOAP) study criteria (Vincent et al. Crit. Care Med. 1998; 26:1793-1800 and Vincent et al. Intensive Care Med. 1996; 22:707-10). 90-day mortality was chosen based on international expert panel recommendations for clinical trials in sepsis (Angus et al. Intensive Care Med. 2003; 29:368-77 and Cohen et al. Crit. Care Med. 2001; 29:880-86). Study nurses ascertained deaths in hospital. Post discharge deaths were ascertained by telephone and National Death Index search. All data collection procedures were conducted under strict confidentiality, audited, and reviewed for accuracy.

Laboratory Procedures

For MIF genotyping, genomic DNA was extracted using QIAamp DNA Blood Mini Kits (QIAGEN Ltd., UK.) and genotyped using previously described techniques with error rates of 1.4% and 0.6% for the MIF −173 G/C and −794 CATT repeat polymorphisms (Baugh et al. Genes Immun. 2002; 3:170-6 and Radstake et al. Arthritis Rheum. 2005; 52:3020-29). For cytokine measurement, plasma TNF, IL-6, and IL-10 concentrations were measured daily for the first week in the hospitalized subjects using an automated chemiluminescent immunoassay analyzer (Diagnostic Products Corp., Los Angeles, Calif.). Day 1 plasma MIF concentrations were measured in 48 subjects (homozygotes matched by age, gender, and comorbidity) using an ELISA assay (R & D System, Minneapolis, Minn.). Day 1 plasma procalcitonin concentrations were measured using a time resolved amplified cryptate emission assay (BRAHMS, Hennigsdorf, Germany) (Christ-Crain et al. Lancet 2004; 363:600-7).

Statistical Analyses

Univariate analyses were performed using Chi-square, Fisher's exact test when necessary, and Armitage's trend test. Two-sample t-tests, analysis of variance, and their nonparametric counterparts were used, when appropriate. Hardy-Weinberg equilibrium was estimated using exact tests. Genotype analyses were performed to ascertain the association of the MIF polymorphisms with CAP susceptibility and outcomes. For the MIF −173 G/C polymorphism, the presence of C allele was also tested, based on prior associations with higher systemic MIF concentrations (Donn et al. Arthritis Rheum. 2004; 50:1604-10 and Donn et al. Arthritis Rheum. 2002; 46:2402-9). Logistic regression models were used to adjust for potential confounders for susceptibility to severe sepsis. Kaplan-Meier plots with log rank test were constructed and Cox regression models were used for survival analyses. Haplotypes were constructed and exact P values were used to assess the association between individual haplotypes and outcomes. Association between individual genotypes and cytokine concentrations was tested using log transformed data and mixed models to evaluate concentrations over time (Laird et al. Biometrics 1982; 38:963-74). Tobit models were used to account for censoring when concentrations were below assay detection limits (Epstein et al. Am. J. Hum. Genet. 2003; 72:611-20). Sensitivity analyses were performed using more stringent definitions of CAP, restricting to microbiologically confirmed cases and using circulating procalcitonin concentrations to classify subjects as having low (<0.1 ng/ml), intermediate (0.1-0.5 ng/ml), and high (>0.5 ng/ml) probability of bacterial pneumonia (Christ-Crain et al. Lancet 2004; 363:600-7). Analyses were performed assuming significance at p<0.05 and using SAS 9.1 and SAS genetics (SAS, Cary, N.C.). Hazard ratios (BR) and odds ratios (OR) are shown with 95% confidence intervals.

Results

Baseline Characteristics

Of the 1738 non-Hispanic white participants with CAP, results for the MIF polymorphisms were available in 1673 (96.5%) participants. Table 6 describes their clinical characteristics. One hundred and ninety eight (11.8%) were discharged after treatment in the EDs. Among the remaining 1475 participants, the incidence of severe sepsis was 32.3%. The 90-day mortality rates for those discharged from EDs, hospitalized subjects, and those with severe sepsis were 1.5%, 12.7% and 27.5%, respectively.

Susceptibility to Community-Acquired Pneumonia

Genotype frequencies of the MIF-173 G/C and -794 CATT repeat polymorphisms in the healthy controls (n=639) and CAP cases are shown in Table 7. No deviation from Hardy Weinberg equilibrium was seen within either group. There were no differences in the genotype frequency distributions between the healthy controls and subjects with CAP for either polymorphism. Differences were not observed in the genotype frequency distributions after stratifying the CAP cases by those discharged from the ED and those requiring hospitalization.

TABLE 6
Clinical and demographic characteristics.

| | | | Hospital | In-patients with severe | In-patients without severe |
|---|---|---|---|---|---|
| Characteristics | All | ED discharges | admissions | sepsis | sepsis |
| N | 1673 | 198 | 1475 | 477 | 998 |
| Age (years): mean (SD) | 68.6 (17.1) | 52.3 (19) | 70.7 (15.6) | 73.3 (15) | 69.5 (15.7) |
| Sex (male): n, % | 887, 53 | 98, 49.5 | 789, 53.5 | 286, 60 | 503, 50.4 |
| Charlson co-morbidity index²³ | | | | | |
| Mean (SD) median | 1.8 (2.2) 1 | 0.6 (1.1) 0 | 1.9 (2.2) 1 | 2.2 (2.3) 1 | 1.8 (2.1) 1 |
| Index > 0: n, % | 1157, 69.2 | 75, 37.9 | 1082, 73.4 | 360, 75.5 | 722 (72.3) |
| Pneumonia Severity Index²¹ | | | | | |
| Mean (SD) median | 98.3 (39.6) | 55 (26.3) 51 | 104.2 (37.4) 100 | 123.3 (39.9) 118 | 95 (32.5) 93 |
| | 96 | | | | |
| Class: n, % | | | | | |
| I, II | 416, 24.9 | 150, 75.8 | 266, 18 | 38, 8 | 228, 22.9 |
| III | 327, 19.6 | 25, 12.6 | 302, 20.5 | 60, 12.6 | 242, 24.3 |
| IV | 599, 35.7 | 21, 10.5 | 578, 39.2 | 190, 39.8 | 388, 38.8 |
| V | 331, 19.8 | 2, 1 | 329, 22.3 | 189, 39.6 | 140, 14 |
| APACHE III score¹: mean (SD) | 54.2 (19.5) | 30.3 (17.7) | 57.4 (17.3) | 66 (19.3) | 53.3 (14.6) |
| Microbiology²: n, % | | | | | |
| Streptococcus pneumoniae | 64, 3.8 | 0 | 64, 4.3 | 22, 4.6 | 42, 4.2 |
| Gram positive pathogen | 188, 10.8 | 0 | 188, 12.8 | 73, 15.3 | 115, 11.5 |
| Gram negative pathogen | 36, 2.2 | 0 | 36, 2.4 | 13, 2.7 | 23, 2.3 |
| Mixed gram positive and gram | 19, 1.1 | 0 | 19, 1.3 | 3, 0.6 | 16, 1.6 |
| negative pathogen | | | | | |
| Chlamydia or Legionella | 5, 0.3 | 0 | 5, 0.3 | 4, 0.8 | 1, 0.1 |

¹APACHE III score assessed on first hospital day, regardless of whether subject was admitted to ICU or not
²Microbiology based cultures from an adequate sputum specimen (<10 squamous epithelial cells and >25 white blood cells per low power field), blood, or bronchoalveolar lavage fluid.

There was evidence for linkage disequilibrium between the MIF-173 G/C and -794 CATT repeat polymorphisms (p<0.001 for both CAP cases and healthy controls). Haplotypes based on the MIF-173 G/C and -794 CATT repeat polymorphisms were constructed (Table 7). Of the 8 possible haplotypes, the C/6, C/7, G/5, and G/6 haplotype frequencies were higher than 1% among controls and CAP cases. The C/7 haplotype was less frequent among subjects with CAP (p=0.04).

TABLE 7
Frequencies of MIF −173G/C, −794CATT repeat polymorphisms,
and haplotypes in community-acquired pneumonia cases and
healthy controls.

| Genotype/ | Cases | Controls | |
|---|---|---|---|
| Haplotype | n (%) | n (%) | Comparisons |
| −173 G/C^{1,3,4} | | | |
| CC | 41 (2.5) | 16 (2.5) | Chi-square for genotype test: |
| | | | p = 0.86 |
| GC | 466 (28.4) | 189 (29.6) | Chi-square for trend test: |
| | | | p = 0.64 |
| GG | 1132 (69.1) | 434 (67.9) | Chi-square for allele test: |
| | | | p = 0.64 |
| −794 CATT | | | |
| repeat^{2,3,4} | | | |
| 5, 5 | 102 (7.1) | 37 (6.2) | Chi-square for genotype test: |
| | | | p = 0.29 |
| 5, 6 | 497 (34.4) | 185 (31.0) | Chi-square for trend test: |
| | | | p = 0.33 |
| 5, 7 | 97 (6.7) | 49 (8.2) | Chi-square for allele test: |
| | | | p = 0.32 |
| 5, 8 | 5 (0.3) | 1 (0.2) | |
| 6, 6 | 536 (37.1) | 243 (40.7) | |
| 6, 7 | 183 (12.7) | 69 (11.6) | |
| 6, 8 | 3 (0.2) | 0 (0) | |
| 7, 7 | 20 (1.4) | 13 (2.2) | |
| −173 G/C | | | |
| haplotype/ | | | |
| −794 CATT | | | |
| repeat^{4,5} | % | % | Overall test p < 0.001 |
| C/6 | 4.9 | 5.0 | P = 0.88 |
| C/7 | 9.5 | 11.8 | P = 0.04 |
| G/5 | 25.7 | 25.1 | P = 0.74 |
| G/6 | 55.9 | 57.1 | P = 0.75 |

¹MIF−173 G/C distribution in Hardy-Weinberg equilibrium for both cases (p = 0.24) and controls (p = 0.49)
²MIF (CATT)ₙ repeat distribution in Hardy-Weinberg equilibrium for both cases (p = 0.26) and controls (p = 0.07)
³No difference in the frequency of either genotypes among CAP requiring hospitalization and CAP discharged from the emergency department
⁴1639 subjects had results for MIF−173 G/C polymorphism, 1443 had results for the −794 CATT repeat polymorphism, and haplotypes were constructed for 1432 subjects with results for both polymorphisms
⁵Haplotypes with prevalence <1% were excluded.

Susceptibility to Severe Sepsis

Genotype frequencies of MIF polymorphisms among subjects hospitalized with CAP, who did and did not develop severe sepsis, are shown in Table 8. There were no statistically significant differences in the genotype frequencies between those who did and did not develop severe sepsis. The observed frequency of severe sepsis was lower in those with the presence of C allele at MIF −173 G/C polymorphism, but results were not significant (OR=0.8 [0.7-1.1], p=0.14). Adjusting for potential confounders, including age, gender, co-morbid conditions, and severity of illness at presentation, did not affect the findings. An increased risk of severe sepsis was not detected for the −794 CATT repeat. Haplotype analysis showed that only the C/6 haplotype was associated with lower risk of severe sepsis (p=0.02) (Table 9).

TABLE 8
Association of MIF promoter polymorphisms with severe sepsis
and 90-day mortality among subjects hospitalized with
community-acquired pneumonia.

| | Severe sepsis | 90-day |
|---|---|---|
| Genotypes | incidence, % | mortality, % |
| 173 G/C | | |
| CC | 25.7¹ | 2.9³ |
| GC | 30.2 | 9.3 |
| GG | 33.5 | 14.4 |
| −794 CATT repeat | | |
| 5, 5 | 31.1² | 13.3⁴ |
| 5, 6 | 29.3 | 10.7 |
| 5, 7 | 34.1 | 15.3 |
| 5, 8 | 40 | 0 |
| 6, 6 | 32.6 | 15.2 |
| 6, 7 | 33.5 | 9.6 |
| 6, 8 | 50 | 0 |
| 7, 7 | 33.3 | 11.1 |

¹Chi-square for genotype test: P = 0.34; Chi-square for trend test: P = 0.14
²Chi-square for genotype test: P = 0.94; P = 0.70; Chi-square for trend test: P = 0.68
³Chi-square for genotype test: P = 0.007; Chi-square for trend test: P = 0.002
⁴Chi-square for genotype test: P = 0.40; Chi-square for trend test: P = 0.51.

TABLE 9
Association of MIF promoter haplotypes¹ with severe sepsis and
90-day mortality among subjects hospitalized with community-acquired
pneumonia.

| | Severe sepsis status² | | | 90-day mortality³ | | |
|---|---|---|---|---|---|---|
| −173 G/C/−794 CATT | With severe | Without severe | | Dead at | Alive at | |
| repeat haplotype | sepsis (%) | sepsis (%) | P value⁴ | 90-days (%) | 90 days (%) | P value⁴ |
| C/6 | 3.0 | 5.5 | 0.02 | 2.4 | 5.0 | 0.07 |
| C/7 | 10.7 | 9.0 | 0.18 | 7.9 | 9.8 | 0.28 |
| G/5 | 25.2 | 25.6 | 0.85 | 25.7 | 25.5 | 0.98 |
| G/6 | 58.1 | 55.1 | 0.20 | 61.9 | 55.1 | 0.03 |

¹Haplotypes with prevalence <1% were excluded
²overall trait test p = 0.02
³overall trait test p = 0.14
⁴P value for individual haplotype trait association.

Survival

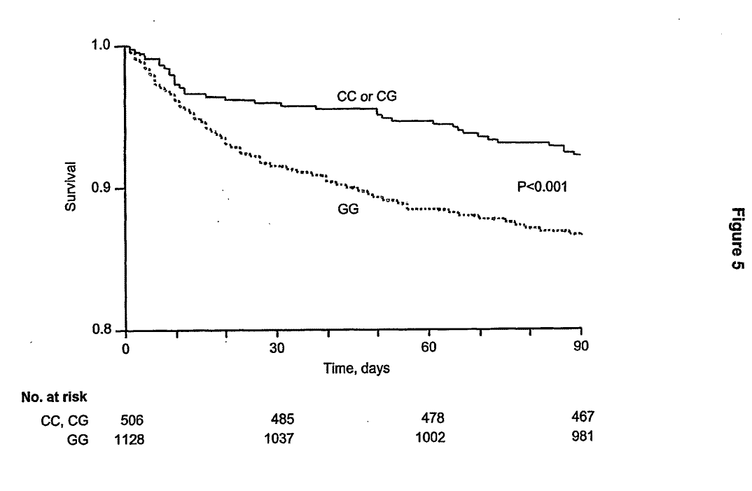

The MIF −173 G/C polymorphism was associated with 90-day mortality (p=0.007), but the MIF −794 CATT repeat was not (Table 8). The presence of the C allele at the MIF −173 site was associated with better 90-day survival (p<0.001) (FIG. 5). The association remained significant in the Cox proportional hazards model that adjusted for age, gender, co-morbid conditions, and day 1 APACHE III score (adjusted HR=0.57 [0.39-0.82], p=0.003). None of the haplotypes were associated with 90-day mortality (p for overall marker trait association=0.14) (Table 9).

Association of MIF-173 G/C Genotypes and Cytokines

Because the MIF-173 G/C genotype was associated with 90-day mortality, the association of this genotype with circulating TNF, IL-6, and IL-10 concentrations was explored in the hospitalized CAP subjects. IL-6 and IL-10 concentrations were higher in subjects with the presence of C allele (p=0.07 and 0.006) on day 1 of hospitalization, but concentrations declined rapidly on day 2 and no differences were seen subsequently. There were no differences in TNF concentrations on day 1 of hospitalization or over the first week (5.7 vs 5.6 pg/ml for the CC and GC genotypes versus GG genotypes, p=0.8, on day 1 of hospitalization). Day 1 circulating MIF concentrations were also determined in a subset of 48 subjects with CC and GG genotypes at the MIF-173 G/C site. A difference was not detected (17.1 vs. 22.1 ng/ml for CC and GG genotypes, p=0.15).

Secondary and Sensitivity Analyses

Subjects with presence of C allele were less likely to develop septic shock (OR=0.5 [0.2-0.9], p=0.03), and there was also a trend towards a reduced likelihood of developing acute renal failure (OR=0.8 [0.6-1.005], p=0.054). There was no difference in susceptibility to other organ failures. The associations between the MIF-173 G/C site and mortality were similar using 30, 60, and 90-day time-points (adjusted OR: 0.5 [0.3-0.9], p=0.02; 0.5 [0.3-0.8], p=0.002, and; 0.6 [0.4-0.9], p=0.01, respectively). Restricting analysis to the 248 (14%) participants hospitalized with CAP who had positive sputum, blood, or bronchoalveolar lavage fluid cultures did not change the findings with regard to susceptibility to CAP. However, the association between the presence of the C allele and reduced risk of severe sepsis became significant (OR=0.4 [0.2-0.8], p=0.006) while the lower risk of death persisted (adjusted HR=0.6 [0.2-1.3], p=0.2). Stratifying by procalcitonin concentration did not affect the findings with regard to susceptibility and severe sepsis. However, the lower risk of death appeared to be restricted to those with intermediate and high probability of bacterial infection (adjusted HRs for C allele within low, intermediate, and high procalcitonin groups: 1.1 [0.4-3], p=0.8; 0.5 [0.2-1.3], p=0.2, and; 0.6 [0.3-0.97], p=0.04).

These results indicated that a third of subjects hospitalized with CAP had a C allele at the MIF-173 G/C site and had a marked reduction in the risk of death independent of co-morbid conditions and severity of illness. The association remained unchanged using different definitions of CAP and using 30, 60, and 90-day time points for mortality. The presence of the C allele was also associated with higher circulating cytokines on admission and the C/7 and C/6 haplotypes were associated with decreased susceptibility to CAP and risk of severe sepsis. Our results differ from previous reports in inflammatory arthritis, where these same genotypes and haplotypes were also associated with higher circulating MIF concentrations but increased susceptibility to and severity of arthritis (Baugh et al. Genes Immun 2002; 3:170-6 and Radstake et al. Arthritis Rheum 2005; 52:3020-29). Our results suggest that an early potent MIF response may be protective in sepsis. Our findings are counter to some of the prior work in animal models of sepsis (Calandra et al. Nat Med 2000; 6:164-70). Therefore, our results not only have important consequences for understanding the precise role of MIF in the inflammatory response to infection, but also on the potential consequences of neutralizing MIF in inflammatory arthritis and severe infection.

For the main outcomes of severe sepsis and mortality, we used an inception cohort design, which is generally considered a stronger approach than a case control design. We recruited a large number of subjects from multiple centers, using recruitment procedures and entry criteria that have been used in previous large federal studies of CAP (Fine et al. N. Engl. J. Med. 1997; 336:243-50). The laboratory staff who performed genotyping were blinded to clinical data and we took a variety of quality control steps to ensure accuracy of laboratory techniques. Spurious associations can certainly be found between genetic polymorphisms and clinical outcomes. However, in this instance, the associations with both outcome measures, mortality and severe sepsis, were in the same direction, the statistical significance of the association with mortality was high, and persisted even after adjusting for potential confounders. Furthermore, findings were unchanged or strengthened when analyses were repeated using more stringent definitions for CAP and infection. As part of on-going work, we have been exploring genetic variation in several other genes involved in the inflammatory response in the same cohort. The work done thus far suggests that the association between the MIF gene and survival is preserved even after adjusting for false discovery and exploring multiple gene effects (data not shown) (Reiner et al. Bioinformatics 2003; 19:368-75).

The MIF −173 G/C polymorphism was associated with survival, but no association was seen with the −794 CATT repeat. In contrast, the C/7 haplotype was associated with decreased CAP susceptibility and the C/6 haplotype was associated with lower risk of severe sepsis, suggesting that both markers within the MIF gene play an important role in susceptibility to and outcomes of CAP.

We measured higher circulating IL-10 levels and detected a trend toward higher circulating IL-6 concentrations on day 1 of hospitalization for subjects with the C allele at the −173 position. Although we were unable to detect differences in MIF concentrations in the small subset of subjects that we analyzed, the pattern of circulating IL-6 and IL-10 concentrations is in accord with the current paradigm that a healthy host response to infection is characterized by a brisk initial cytokine response followed by a rapid decline (Hotchkiss et al. N. Engl. J. Med. 2003; 348:138-150). We speculate that the elevated day 1 cytokines observed in subjects with the protective MIF genotypes may be a downstream consequence of a surge in MIF release prior to hospitalization.

In conclusion, the presence of a C allele at the MIF −173 G/C site was associated with improved survival after CAP among whites in a large multicenter cohort.

### Example 4

### Functional Promoter Polymorphisms in the MIF Gene in Patients with Streptococcus pneumoniae Bacteremia and Meningitis

S. pneumoniae causes a spectrum of disease severity, and human host factors likely play a role in this variation. It was examined whether high-expressing MIF alleles (e.g., MIF alleles with >5 CATT repeats in the −794 region of the MIF promoter) were associated with severe invasive pneumococcal disease, including meningitis. Blood samples and patient chart findings were collected prospectively at three Connecticut hospitals from 24 inpatients with documented invasive S. pneumoniae infections. Genomic DNA was isolated from blood, amplified, and genotyped using optical biosensor chips. Fisher's exact tests were used to compare subjects with the high-expressing 7,7 CATT genotype to all other genotypes.

Of the 24 bacteremic subjects, 18 (75%) had pneumonia, 2 (18%) had meningitis, 3 (13%) had meningitis and pneumonia, and 1 (4%) had an unknown focus. Genotypes were completed for each allele of 9 of the bacteremic subjects. Compared to normal healthy control samples previously analyzed, the genotype frequences were observed as in the table below:

TABLE 10
Association of MIF promoter haplotype with meningitis.

| | MIF repeat # in each allele | | | | | | | | |
|---|---|---|---|---|---|---|---|---|---|
| | 5, 5 | 6, 6 | 7, 7 | 5, 6 | 5, 7 | 6, 7 | 5, 8 | 6, 8 | 7, 8 |
| Control, | 11 (6) | 62 (33) | 2 (1) | 69 (37) | 13 (7) | 29 (15) | 1 (1) | 1 (1) | 1 (1) |
| n = 189 (%) | | | | | | | | | |
| Bacteremia | 1 (20) | 2 (40) | 0 | 2 (40) | 0 | 0 | 0 | 0 | 0 |
| alone, n = 5 | | | | | | | | | |
| (%) | | | | | | | | | |
| Meningitis, | 0 | 1 (25) | 2 (50)* | 1 (25) | 0 | 0 | 0 | 0 | 0 |
| n = 4 (%) | | | | | | | | | |

*p = 0.019 compared with healthy controls.

Patients with pneumococcal meningitis were found to have a higher frequency of the rare 7,7 CATT genotype (n=2/4, 50%) compared with patients with bacteremia alone (n=0/5, 0%) or control subjects (n=2/189, 1.1%).

### Example 5

### Downregulation of CCR5 by MIF and Protection of HIV Infection by MIF Agonists

MIF is a Component of Innate HIV Immunity.

The production of MIF by monocyte-derived macrophage (MDM) cultures in response to HIV-1 infection was examined. Triplicate cultures of monocyte-derived macrophages were infected with HIV-1_{ADA} and cultivated until infection reached its peak (day 12, RT activity 10,500 cpm/μl). Cells were washed, and cultured in fresh medium. Aliquots of culture medium were withdrawn on the indicated days and analyzed by MIF-specific ELISA. Statistical analysis using Student's t-test demonstrated significant differences between the amounts of MIF produced by mock-infected and HIV-infected cultures, p<0.05. MIF levels in MDM cultures infected with HIV-1_{ADA} were significantly increased compared to mock-infected cultures (FIG. 6). The time course of MIF production coincided with HIV-1 replication (maximum MIF levels were observed at the peak of infection).

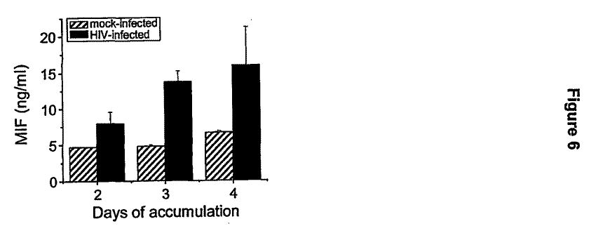

The kinetics of MIF production by MDM cultures mimicked that of production of another immunomodulatory cytokine, chemokine MIP-1α (Schmidtmayerova et al. (1996). Proc. Natl. Acad. Sci. U.S.A. 93:700-704), suggesting that MIF may be made as a protective innate response of the cells to HIV infection. To investigate this possibility, HIV-infected macrophages were cultured in the presence of a neutralizing anti-MIF antibody or isotype control. Triplicate cultures of monocyte-derived macrophages were infected with HIV-1_{ADA} in the presence of anti-MIF MAb (25 μg/ml) or isotype control. Virus replication was monitored by RT activity in culture supernatants. A significant increase in replication of the CCR5-tropic (R5) HIV-1 strain ADA was observed in the presence of anti-MIF antibody (FIG. 7A). This finding was confirmed by experiments using recombinant MIF, which significantly suppressed replication of HIV-1 in macrophage cultures (FIG. 7B). The observed effect was not due to the presence of endotoxin in recombinant MIF, as addition of an LPS inhibitor, polymyxin B (10 μg/ml), did not significantly reduce the inhibitory effect of MIF on viral replication (FIG. 7B). In addition, the effect of MIF was reduced by the antibody to the MIF receptor, CD74 (FIG. 7C). Macrophages were infected with HIV-1_{ADA} and cultured in the presence of MIF (50 ng/ml) mixed with PMB (10 μg/ml) and anti-CD74 MAb (25 μg/ml). Control cultures were cultivated with PMB mixed with an isotype immunoglobulin with or without MIF. These data indicate that MIF is a component of innate anti-HIV immunity.

MIF Inhibits Replication in PBMC of R5, but not X4, HIV-1 Strain.

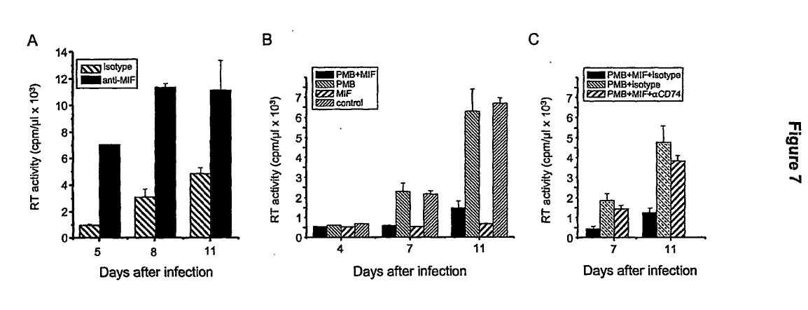

Triplicate PHA-activated PBMC cultures were infected with R5 (ADA) or X4 (LAI) strains of HIV-1 and cultivated in the presence or absence (control) of recombinant MIF (50 ng/ml). At indicated time points after infection, reverse transcriptase activity was measured in culture supernatants. Whereas differences between MIF-treated and control cultures of ADA-infected PBMCs are highly statistically significant (p<0.01), differences between MIF-treated and control cultures of LAI-infected cells are not significant (p>0.05) (FIG. 8).

Similar to its effect on replication of R5 HIV-1 in macrophage cultures, recombinant MIF reduced R5 HIV-1 replication in PBMC cultures. MIF did not reduce replication of the CXCR4-dependent strain LAI. To test the effect of MIF on CCR5 expression, MDM cultures were treated with recombinant MIF. The expression of CCR5 and CXCR4 was measured by flow cytometry. This experiment demonstrated that MIF substantially reduces CCR5 expression, without altering expression of CXCR4 (FIG. 9). This result is consistent with observed inhibition of R5 HIV strains by MIF.

MIF Downregulates CCR5 Expression in Macrophages.

Monocyte-derived macrophages were cultured in the presence of recombinant MIF (50 ng/ml) and polymyxin B (PMB, 10 μg/ml) for 48 h. Cell surface expression of CCR5 and CXCR4 was analyzed by FACS after staining with FITC-conjugated anti-CCR5 and PE-conjugated anti-CXCR4 antibodies (Pharmingen) (FIG. 9).

The Role of Nef in the Regulation of MIF Secretion During HIV Infection

A recent report demonstrated that secretion of MIF may be mediated by ABCA1, an ATP-binding cassette transporter (Flieger et al. (2003). FEBS Lett. 551:78-86). In support of the role of Nef in regulation of MIF secretion, our preliminary results show that HIV-1 infection of human monocyte-derived macrophages or transfection of murine macrophage cell line, RAW 264.7, with a Nef-expressing plasmid leads to increased localization of ABCA1 at the plasma membrane. Therefore, Nef may stimulate MIF release from HIV-infected macrophages by increasing localization at the plasma membrane of ABCA1. On day 5 after infection with Nef-expressing or Nef-deficient HIV-1, cells were co-stained with anti-p24 mouse monoclonal and anti-ABCA1 rabbit polyclonal antibodies, followed by Rhodamine-conjugated anti-mouse and Cy5-conjugated anti-rabbit IgG.

### Example 6

### Identification of CD44 as a Signaling Component of the MIF/CD74 Receptor Complex

In this Example, we demonstrate that MIF signaling through CD74 requires the simultaneous expression and activation of CD44. We performed studies in cell lines engineered to stably-express CD74 or CD44, their combination, or CD74 together with a truncated CD44 lacking its cytoplasmic signaling domain (CD44^{Δ67}). We also investigated the responses of primary cells genetically-deficient in CD74 or CD44. Our findings provide evidence for a direct role for CD44 and the CD44-associated src kinase, p56^{lck}, in MIF-mediated ERK-activation and protection from apoptosis.

Creation and Characterization of Stably-Expressing CD74 and CD44 Transformants

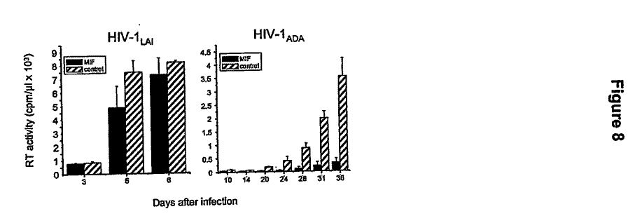

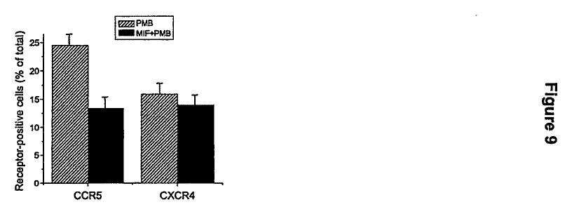

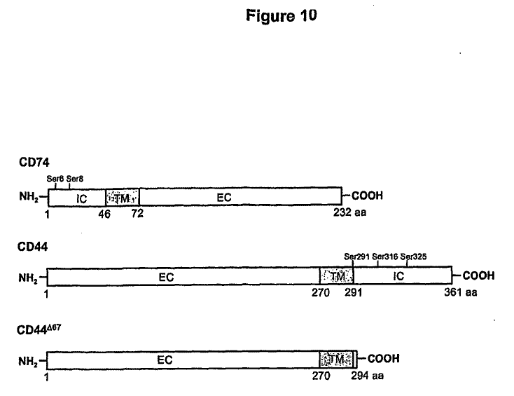

Stable cell lines expressing combinations of CD74 and different forms of CD44 were created to investigate the roles of CD74 and CD44 in MIF signal transduction. Mammalian COS-7 cells do not bind MIF unless engineered to express CD74 (Leng et al. (2003). J Exp Med 197:1467-1476), and the COS-7/M6 subline additionally is known to be CD44^{null} (Jiang et al. (2002). J Biol Chem 277:10531-10538). The absence of CD74 and CD44 was confirmed in COS-7/M6 cells by western blotting, and the cells then were transfected with plasmid DNA encoding full-length human CD74 (1-232 aa), full-length CD44 (1-361 aa of the hematopoietic “H” isoform of CD44), or a truncated CD44 lacking its cytoplasmic domain (CD44^{Δ67}) (FIG. 10). Cell lines expressing the corresponding cDNAs were propagated and selected for further study based on the stable expression of these proteins.

The cell surface expression of the transfected proteins was confirmed by flow cytometry using directly conjugated (FITC-labeled) anti-CD74 and anti-CD44 antibodies. Equivalent surface expression of the full-length and truncated forms of CD44 was verified—which is important for functional analyses. It was also verified that expression of the MIF binding receptor, CD74, was not influenced by the presence of CD44 or CD44^{Δ67}.

These stable cell lines were utilized to assess the potential contribution of CD44 to the MIF binding interaction with CD74. There is evidence that CD44 may associate with CD74 (Naujokas et al. (1993). Cell 74:257-268 and Meyer-Siegler et al. (1996). Urology 48:448-52), and it was hypothesized that CD44 may act as a co-receptor to enhance MIF binding to the cell surface. An Alexa-488, fluorescently-labeled MIF was prepared (used previously in the expression cloning of CD74 (Leng et al. (2003). J. Exp. Med. 197:1467-1476)). Its binding activity was tested with each of the different transfected cell lines by flow cytometry. These results support the conclusion that CD74 alone is sufficient to mediate MIF binding to cells. CD44 alone does not bind MIF, nor does CD44 confer additional binding above that provided by CD74 alone.

CD44 is Required for MIF-Mediated ERK-1/2 Phosphorylation

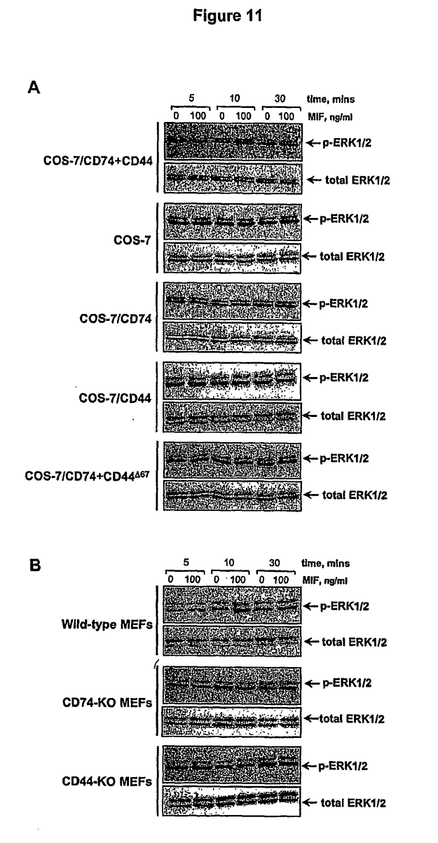

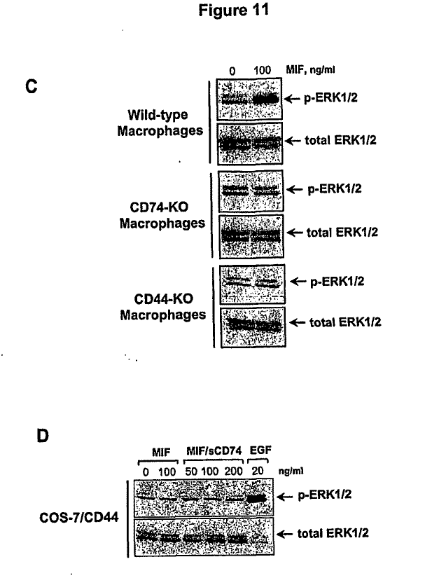

The phosphorylation and activation of the ERK-1/2 (p42/p44) subfamily of MAP kinases is an established feature of MIF signal transduction (Mitchell et al. (1999). J Biol Chem 274:18100-6; Lacey et al. (2003). Arthritis Rheum 48:103-109; and Amin et al. (2003). Circ Res 93:321-329). ERK-1/2 phosphorylation was examined in response to a stimulatory concentration of MIF (100 ng/ml) in the transfected cell lines over time. MIF induced ERK-1/2 phosphorylation only in those cells expressing both CD74 and full-length CD44 (FIG. 11A). ERK-1/2 phosphorylation was induced as early as 10 mins, which is in accord with prior studies (Mitchell et al. (1999). J. Biol. Chem. 274:18100-6). While MIF can induce a sustained pattern (>90 mins) of ERK-1/2 phosphorylation in some cell types (Mitchell et al. (1999). J. Biol. Chem. 274:18100-6 and Liao et al. (2003). J. Biol. Chem. 278:76-81, this was not observed in the stable COS-7 expression system. ERK-1/2 phosphorylation decayed at 30 mins.

The requirement for CD44 in MIF signal transduction was verified by examining ERK-1/2 phosphorylation in primary, murine embryonic fibroblasts (MEFs) (FIG. 11B), and in peritoneal macrophages prepared from wild-type, CD74-KO, and CD44-KO mice (FIG. 11C). MIF induced ERK-1/2 phosphorylation only in wild-type cells, and not in cells genetically-deficient in CD74 or CD44.

The CD44 requirement for MIF signal transduction may be due to CD44 binding to a MIF that has undergone conformational modification by complexation with CD74. As a test of this possibility, a soluble, recombinant CD74 ectodomain (sCD74) was prepared, which binds MIF with a Kd ˜9×10⁻⁹ (Leng et al. (2003). J. Exp. Med. 197:1467-1476). Increasing concentrations of pre-formed MIF/sCD74 complexes were added to cells expressing CD44 alone (FIG. 11D). No increase in ERK-1/2 phosphorylation was observed under these conditions, supporting the requirement for an interaction between membrane-expressed CD74 and CD44. These data, taken together, are consistent with the interpretation that MIF-induced ERK-1/2 phosphorylation requires the cellular expression of two integral membrane proteins: a binding receptor (CD74), and a signaling protein (CD44).

MIF Modulates the Serine Phosphorylation of CD74 and CD44

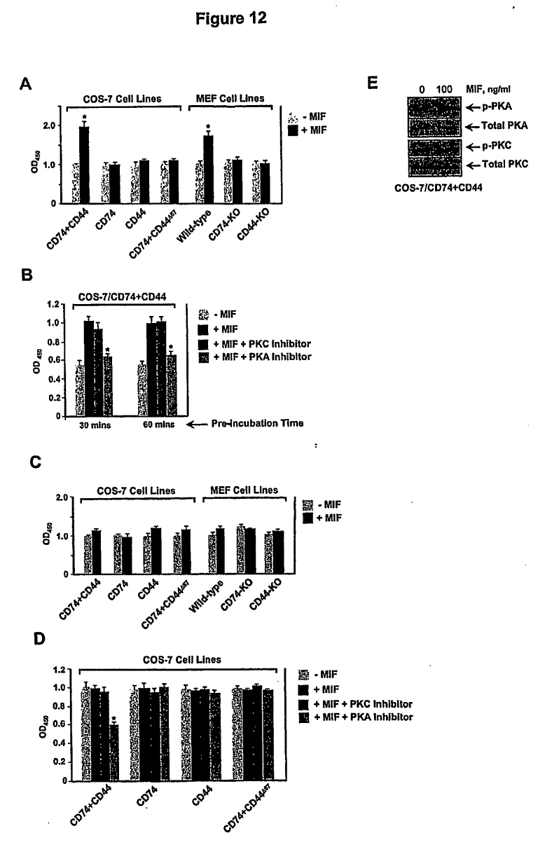

Two serine residues within the CD74 intracytoplasmic domain (Ser6 and Ser8) undergo phosphorylation in a protein kinase-dependent manner (Anderson et al. (1999). J. Immunol. 163:5435-5443), although the role of an activating ligand such as MIF in CD74 phosphorylation has not been considered previously. The content of phospho-serine residues in CD74 was quantified by a specific sandwich ELISA (Perez et al. (2003). Nature Immunol. 4:1083-92) before and after MIF stimulation. As shown in FIG. 12A, the phosphorylation of CD74 in the COS-7/CD74+CD44 cell line and in wild-type MEFs (CD74⁺/CD44⁺) increased significantly in the presence of MIF, but it did not change in the other COS-7 transformants and genetically-deficient, primary cell lines. The observation that the phosphoserine content of CD74 increased only in cells that expressed CD74 and full-length CD44 is consistent with the notion that the MIF-mediated phosphorylation of CD74 is dependent on the activity of the CD44 intracytoplasmic domain.

Protein kinase A (PKA) has been implicated in the MIF-dependent phosphorylation of ERK-1/2 leading to downstream activation events (Mitchell et al. (1999). J Biol Chem 274:18100-6). The potential role of PKA and PKC was tested, which may phosphorylate CD74 in vitro (Anderson et al. (1999). J. Immunol. 163:5435-5443), by analyzing MIF-induced CD74 phosphorylation in the presence of the PKA inhibitor, H-89, or the PKC inhibitor, RO-31-2880. MIF stimulation of CD74 phosphorylation decreased markedly upon PKA inhibition. No effect was seen in the presence of the PKC inhibitor RO-31-2880 at the effective and PKC-selective concentration of 10 μM (Padfield et al. (1998). Biochem. J. 330:329-334 and Hill et al. (2003). British J. Pharmacol. 139:721-732) (FIG. 12B). These results suggest that the MIF-dependent, serine phosphorylation of CD74 proceeds via PKA.

The effect of MIF stimulation on the phosphorylation of CD44 was analyzed, which may occur on three intracytoplasmic serine residues (Ser291, Ser316, and Ser325) (Ponta et al. (2003). Nature Revs. Molr. Cell Biol. 4:33-45 and Thorne et al. (2004). J. Cell Science 117:373-380). Ser325 is constitutively phosphorylated in resting cells, but undergoes de-phosphorylation in response to PKC activating stimuli such as phorbol esters. Conversely, Ser291 and Ser316 are unphosphorylated in resting cells, but then may be phosphorylated by the activation of PKC (Ser291) and PKA (Ser316) (Legg et al. (2002). Nature Cell Biol 4:399-407; Ponta et al. (2003). Nature Revs. Molr. Cell Biol. 4:33-45; and Thorne et al. (2004). J Cell Science 117:373-380). The effect of MIF-stimulation CD44 phosphorylation was analyzed in the COS-7 and MEF cell lines by sandwich ELISA using an anti-CD44 capture antibody to quantify CD44 phospho-serine residues (Perez et al. (2003). Nature Immunol. 4:1083-92). In contrast to the phosphoserine content of CD74, which increased in response to MIF, the net content of CD44 serine phosphorylation did not change after MIF stimulation (FIG. 12C). This result may be due to either the lack of effect of MIF on CD44 serine phosphorylation or, alternatively, to a mixed effect on CD44 phosphorylation and de-phosphorylation such that net phosphoserine content did not change. Evidence for this second possibility was obtained by analyzing CD44 phosphoserine content after stimulation in the presence of the protein kinase inhibitors. Under these conditions, the serine phosphorylation of CD44 was reduced by PKA inhibition (H-89, 20 μM), but not by PKC inhibition (RO-31-2880, 10 μM) (FIG. 12D). A role for MIF activation of PKA was confirmed by western blotting of the COS-7/CD74+CD44 cell line, which showed MIF-dependent phosphorylation of PKA but not PKC (FIG. 12E). These results suggest that MIF stimulation is indeed associated with a PKA-dependent alteration in the serine phosphorylation of CD44, but because the net phosphoserine content is unaltered, there is a reciprocal decrease in the level of constitutively phosphorylated serines. There is precedent for reciprocal changes in the serine phosphorylation of CD44 upon protein kinase activation, with a pattern involving a PKA-mediated increase in Ser316 phosphorylation accompanied by the de-phosphorylation of Ser325 m (Ponta et al. (2003). Nature Revs. Molr. Cell Biol. 4:33-45 and Thorne et al. (2004). J. Cell Science 117:373-380).

The Protein Tyrosine Kinase p56^{lck} is Activated by MIF Engagement of the CD74/CD44 Complex

Two members of the non-receptor, protein tyrosine kinase family, p56^{lck} and c-Src (Src), may physically associate with the intracytoplasmic domain of CD44 (Elaimin I. Taher (1996). J. Biol. Chem. 271:2863-2867 and Bourguignon et al. (2001). J. Biol. Chem. 276:7327-7336), and either kinase may lead to downstream ERK1/2 phosphorylation via a Ras-Raf-MEK-dependent pathway (Ishida et al. (1998). Circ Res 82:7-12; Migliaccio et al. (2000). EMBO J. 19:5406-5417; and Mahabeleshwar et al. (2003). J. Biol. Chem. 278:52598-52612). The phosphorylation state of p56^{lck} and Src was analyzed in the different COS-7/M6 transformants and MEFs after MIF stimulation by immunoblotting with pairs of anti-phospho-p56^{lck} and anti-total p56^{lck} antibodies, and anti-phospho-Src and anti-total Src antibodies. p56^{lck} was phosphorylated by MIF treatment, but only in the cells expressing both CD74 and full-length CD44. The level of p56^{lck} phosphorylation appeared less in the MEFs than in the COS-7 cells, which may be attributed to the lower level of expression of the p56^{lck} kinase in cells of fibroblast lineage (Oikawa et al. (2001). Immunol. 104:162-167), or to a higher activation and coupling efficiency of CD44 when over-expressed in COS-7 cells. An increase in the phosphorylation of the Src kinase was not detected in either cell type however, suggesting that this kinase is not involved in the CD44-dependent, signal transduction of MIF.

To confirm a role for p56^{lck} in MIF signal transduction leading to ERK1/2 phosphorylation, a short interfering RNA (siRNA) directed against p56^{lck} (Csk2033) was prepared, together with a mutant and control siRNA (Csk2033-M3) (Iversen et al. (2004). FASEB J 18, C258). Their ability to prevent the MIF-stimulated phosphorylation of p56^{lck} and ERK-1/2 was tested. Treatment of the COS-7/CD74+CD44 cells with the p56^{lck}-specific siRNA (Csk2033) but not the control siRNA (Csk2033-M3) reduced intracellular p56^{lck} levels and decreased MIF-induced ERK1/2 phosphorylation. The potential effect of the kinase inhibitor, damnacanthal, was also tested, which at a concentration of 20 nM inhibits p56^{lck} activity without affecting other Src kinase family members (Faltynek et al. (1995). Biochemistry 34:12404-12410). Damnacanthal addition to the COS-7/CD74+CD44 cell line or to primary macrophages resulted in an inhibition of the phosphorylation of p56^{lck} and ERK1/2. These data, taken together, are consistent with a role for the CD44-associated kinase, p56^{lck}, in MIF signal transduction leading to ERK1/2 phosphorylation.

CD74 and CD44 are Required for MIF-Mediated Protection from p53-Dependent Apoptosis

An important biologic action of MIF is to sustain pro-inflammatory responses by inhibiting activation-induced, p53-dependent apoptosis (Hudson et al. (1999). J. Exp. Med. 190:1375-1382; Mitchell et al. (2002). Proc. Natl. Acad. Sci. USA 99:345-350; and Nguyen et al. (2003). J. Immunol. 170:3337-3347). MIF reduces p53 accumulation in the cytoplasm by a pathway that requires an ERK1/2 effector response leading to arachidonic acid metabolism and cyclooxygenase-2 activation (Hudson et al. (1999). J. Exp. Med. 190:1375-1382 and Mitchell et al. (2002). Proc. Natl. Acad. Sci. USA 99:345-350). CD74 and CD44 were tested for whether they were necessary for this action of MIF by assessing the apoptotic response of the different COS-7 cell lines and primary macrophages genetically-deficient in CD74 or CD44. The COS-7 cell lines showed a brisk response to apoptotic induction, but only in the case of the COS-7/CD74+CD44 cell line did MIF exert a significant protective effect. The anti-apoptotic action of MIF in turn was associated with a reduction in the intracytoplasmic content of a Ser15-phosphorylated, p53 species, as reported previously (Mitchell et al. (2002). Proc. Natl. Acad. Sci USA 99:345-350). MIF protection from apoptosis of primary macrophages also was found to be dependent on CD74 and CD44, and in these cells the protective effect of MIF was almost complete, which is consistent with prior reports (Hudson et al. (1999). J Exp Med 190:1375-1382 and Mitchell et al. (2002). Proc. Natl. Acad. Sci. USA 99:345-350). MIF treatment of macrophages during apoptosis induction also was associated with a diminution in the cellular content of Ser15-phosphorylated, p53.

The impact of the MIF-CD74/CD44 signal transduction pathway was analyzed in vivo by examining macrophage apoptosis in endotoxemic mice. Wild-type, MIF-KO, CD74-KO, and CD44-KO mice were primed with endotoxin (LPS) and their macrophages harvested one day later by peritoneal lavage. Initial assessment of macrophage viability by fluorescent-annexin staining showed that endotoxemic, wild-type mice had a several-fold increase in apoptotic macrophage numbers when compared to saline-treated controls. The apoptotic response was enhanced in the MIF-KO mice when compared to the wild-type controls, which is in agreement with a prior report (Mitchell et al. (2002). Proc. Natl. Acad. Sci. USA 99:345-350), and apoptosis also appeared increased in the CD74-KO and the CD44-KO mouse strains. A more quantitative assessment of apoptosis in macrophages was obtained by oligonucleosome ELISA. This analysis showed an equivalent level of LPS-induced apoptosis in macrophages isolated from the MIF-KO, CD74-KO, and CD44-KO strains that in turn was enhanced when compared to wild-type controls. These data confirm the role of CD74 and CD44 in the MIF-mediated protection of macrophage apoptosis in vivo.

The respective binding and signaling roles for CD74 and CD44 are reminiscent of the signaling mechanism established for the IL-6 family of cytokines, whereby a binding receptor (i.e. IL-6Rα) associates with a transmembrane glycoprotein (i.e. gp130) leading to kinase activation (Hibi et al. (2001). IL-6 Receptor. In Cytokine Reference, Vol 2: Receptors, J. J. Oppenheim and M. Feldmann, eds. (San Diego: Academic Press), pp. 1761-1778). Several of the biologic activities of MIF have been identified to proceed via ERK1/2 activation. These include arachidonic acid metabolism and prostaglandin production (via cytoplasmic phospholipase A2 and cyclooxygenase-2) (Mitchell et al. (1999). J. Biol. Chem. 274:18100-6 and Sampey et al. (2001). Arthritis Rheum. 44:1273-1280), regulation of p53 activity (Mitchell et al. (2002). Proc. Natl. Acad. Sci. USA 99:345-350), and the activation of additional ERK1/2 effectors, such as the Ets family of transcription factors that regulate TLR4 expression (Roger et al. (2001). Nature 414:920-924).

Materials and Methods

Mice and Primary Cells. Wild-type (BALB/c or C57/B16), CD74-KO, (Shachar et al. (1996). Science 274:106-108), CD44-KO (Teder et al, (2002). Science 296:155-158), and MIF-KO (Bozza et al. (1999). J Exp Med 189:341-346), mice were bred at the Yale Animal Resource Center under strict, pathogen-free conditions. The KO strains were in genetically pure BALB/c or C57/B16 backgrounds (each at generation ≧N10). Mouse embryonic fibroblasts (MEFs) were obtained from 2 week-old embryos as described (Fingerle-Rowson et al. (2003). Proc Natl Acad Sci USA 100:9354-9359). Initial studies revealed no detectable differences in MIF signaling responses of cells derived from mice in the BALB/c versus the C57/1316 background. Primary macrophages from peritoneal exudate fluid was obtained after intraperitoneal (i.p.) injection of mice with 4% thioglycollate (Calandra et al. (1994). J Exp Med 179:1895-902).

Plasmid DNA Vectors. The pcDNA3.1-CD74 plasmid was constructed by inserting a full length, human CD74 cDNA fragment (1-232aa) into the pcDNA3.1/V5-His-TOPO vector (Invitrogen) at a multiple cloning site (Leng et al. (2003). J Exp Med 197:1467-1476). The pTracer-CD44 and pTracer-CD44^{Δ67} plasmids, which encode the human hematopoietic form of CD44 (CD44H, 1-361 aa) and a truncated CD44 lacking the cytoplasmic domain (CD44^{Δ67}, 1-294 aa) respectively, were created by subcloning into the pTracer-SV40 vector (Invitrogen) a human full-length CD44 cDNA or a CD44 cDNA created by substitution of a stop codon for cysteine 295 using site-directed mutagenesis (Jiang et al. (2002). J Biol Chem 277:10531-10538). Structural fidelity was confirmed by DNA sequencing.

Creation of Stable Transformants. COS-7/M6 cells, which express neither CD74 nor CD44 (Jiang et al. (2002). J Biol Chem 277:10531-10538) (null expression was confirmed by flow cytometry and Western blotting), were stably transfected with pcDNA3.1-CD74, pTracer-CD44, pcDNA3.1-CD74 plus pTracer-CD44, or pcDNA3.1-CD74 plus pTracer-CD44^{Δ67} by using the LipofectAMINE PLUS Kit (Invitrogen). Stable transformants were selected by culture in G418 (1.5 mg/ml, Sigma). The different transformants were subcloned and stable expression confirmed periodically by immunoblotting with anti-human CD74 antibody (BD Pharmingen) and anti-human CD44 antibody (sc-7297, Santa Cruz). Clones stably expressing immuno-equivalent levels of CD74 and CD44 were used for functional studies. COS-7M6 cells were used as negative controls, and HeLa cells (Bouchard et al. (1997). J Virol 71:2793-2798) expressing CD44 were used as positive controls.

Flow Cytometry. Washed cells (˜4.0×10⁵) were re-suspended in PBS/2% FBS and stained on ice for 30 minutes with isotypic controls, anti-human CD74, anti-human CD44, or anti-mouse CD44 (BD Pharmingen) antibodies conjugated with FITC or Alexa 488-labeled MIF (Leng et al. (2003). J Exp Med 197:1467-1476). The labeled cells were studied with a FACS-Calibur (BD Pharmingen) and the data were analyzed with CellQuest software (BD Pharmingen).

Signal Transduction Assays. Cells were stimulated with native sequence, recombinant human MIF (for COS-7 transformants) or mouse MIF (for MEFs and macrophages) that was produced in our laboratory (Bernhagen et al. (1994). Biochemistry 33:14144-14155). The human CD74 ectodomain (CD74⁷³⁻²³²) was prepared recombinantly in soluble form (sCD74) as described previously (Leng et al. (2003). J Exp Med 197:1467-1476). In a typical experiment, 2×10⁶ cells cultured in 10 cm plates were rendered quiescent by incubation in low serum for 16 Ins prior to stimulation with MIF for 5-30 mins (Mitchell et al. (1999). J Biol Chem 274:18100-6). Cells then were harvested and lysed in buffer containing 50 mM Hepes, pH7.5, 150 mM NaCl, 1.5 mM MgCl₂, 1 mM EGTA, 1% Glycerol and 1% Triton X-100, and freshly added phosphatase and proteinase inhibitors (1 mM orthovanandate, 10 mM NaF, 10 μg/ml leupeptin, 25 μg/ml aprotinin and 50 μg/ml PMSF). For immunoblotting, 15-20 μg of cell lysate proteins were separated by 10% SDS-PAGE and transferred to PVDF Immobilon-P transfer membranes (Millipore). The blots were developed with specific primary antibodies that included a pair of anti-phospho-ERK-1/2 (sc-7383, Santa Cruz) and anti-(total) ERK-1/2 (sc-94, Santa Cruz) antibodies, a pair of anti-phospho-p56^{lck} (Cell Signaling) and anti-(total) p56^{lck} (sc-433, Santa Cruz) antibodies, or a pair of anti-phospho-Src and anti-(total) Src antibodies (Cell Signaling). The secondary antibodies included anti-mouse IgG or anti-rabbit IgG conjugated with horseradish peroxidase (Cell Signaling). The ECL detection reagents (Amersham) were used to visualize bands. The blots displayed are representative of stimulation studies that were repeated at least three times.

The serine phosphorylation of CD74 and CD44 was quantified by specific sandwich ELISA (Perez et al. (2003). Nature Immunol 4:1083-92). Ninety-six well microtiter plates were coated with 1-2 μg/ml of anti-CD74 or anti-CD44 antibody at 4° C. overnight. Lysates from MIF-stimulated or control cultures were added to antibody-coated wells and incubated at 4° C. overnight. After several washes (0.5% Tween-20 in PBS), 0.2 μg/ml anti-phospho-serine antibody (Hypromatrix) conjugated with horseradish peroxidase was added, and immunoreactivity measured by the TMB reagent (DakoCytomation) and the data analyzed with SpectraMax Plus (Molecular Devices).

Protein kinase inhibition was performed by pre-incubating cells for 30-120 mins with 20 μM of the PKA inhibitor, H-89, 10 μM of the PKC inhibitor, RO-31-2880, or 20 nM of the p56^{lck} inhibitor, damnacanthal (Calbiochem) (Faltynek et al. (1995). Biochemistry 34:12404-12410). Cell lysates were collected and analyzed by immunoblots using a pair of anti-phospho-PICA and anti-(total)-PKA antibodies, a pair of anti-phospho-PKC (all from Cell Signaling) and anti-(total)-PKC (Santa Cruz) antibodies, or a pair of anti-phospho-p56^{lck} (Cell Signaling) and anti-(total) p56^{lck} (sc-433, Santa Cruz) antibodies.

siRNA Studies. An siRNA specific for p56^{lck} mRNA (Csk 2033) and its mutant control (Csk 2033-M3) were prepared as described previously (Iversen et al. (2004). FASEB J 18, C258). Both the siRNAs Csk 2033 and Csk 2033-M3 are two 21-nucleotide double-stranded RNAs with 2-nucleotide 3′ overhangs. In Csk 2033, the sense sequence is 5′-ACUCGCCUUCUUAGAGUUUUA-3′ (SEQ ID NO: 25) and antisense sequence is 5′-AAACUC-UAAGAAGGCGAGUGG-3′ (SEQ ID NO: 26). In Csk 2033-M3, the sense sequence is 5′-ACUCGGCUUGUUAG-ACUUUUA-3′ (SEQ ID NO: 27), and antisense sequence is 5′-AAAGUCUAACAA-GCCGAGUGG-3′ (SEQ ID NO: 28). The Csk 2033 siRNA, but not the Csk 2033-M3 control, reduced p56^{lck} levels significantly (>75%) in mature T cells, as reported previously (Iversen et al. (2004). FASEB J 18, C258). In the present studies, cells were transfected with 800 nM Csk 2033 and Csk 2033-M3 using the siRNA Transfection Kit (Ambion). Forty-eight hours after transfection, the cell lysates were collected and analyzed with immunoblots using anti-human phospho-p56^{lck} and anti-human p56^{lck} antibodies.

Apoptosis Studies. For the in vitro apoptosis studies, 2×10⁶ cells cultured in 10 cm plates were treated for 24 hrs with apoptosis inducers, 1 mM sodium nitroprusside (SNP, Sigma) and 2 μg/ml camptothecin (Sigma) (Hudson et al. (1999). J Exp Med 190:1375-1382 and Mitchell et al. (2002). Proc Natl Acad Sci USA 99:345-350) in the presence or absence of MIF (100 ng/ml). Cell lysates were collected, and caspase-3 activity analyzed using a colorimetric assay kit (R&D Systems). Cytoplasmic p53 content was analyzed as described previously (Mitchell et al. (2002). Proc Natl Acad Sci USA 99:345-350) by immunoblotting of mouse cytosolic fractions using a pair of anti-phospho-p53 (Ser15) (R&D Systems) and total anti-p53 antibodies (Cell Signaling); and for COS-7 cells, the anti-p53 (Ser15) and total anti-p53 antibodies (Cell Signaling). In vivo apoptosis studies followed the procedures of (Mitchell et al. (2002). Proc Natl Acad Sci USA 99:345-350), and were conducted after i.p injection of 15 mg/kg lipopolysaccharide (E. coli 0111:B4; Sigma). Mice were sacrificed 24 hrs after priming and the peritoneal macrophage cells collected and immediately analyzed for apoptosis by an ELISA that detects cytoplasmic oligonucleosomes (Roche Diagnostics). Primary macrophages also were immunostained by Alexa 488-conjugated, Annexin V (Invitrogen), and apoptotic cells enumerated by fluorescence microscopy.

### Example 7

### MIF Genotype and Asthma

MIF's role in asthma was examined using genetic approaches in an experimental mouse model and in a cohort of asthma patients.

MIF^{-/-} Mice Show a Reduced Immunoglobulin Response to OVA.

Murine models of asthma such as the OVA-prime and aerosol challenge model are characterized by the preferential induction of a T_{H}2 immunologic response. A standard protocol was used for OVA-priming to examine first the total and OVA-specific, serum immunoglobulin response in MIF⁴″ mice and genetically-matched, MIF^{+/+} mice. Both total and OVA-specific IgM, IgE, IgG1, and IgG2a increased in serum by day 8 of OVA-sensitization in the wild-type, MIF^{+/+} strain (FIG. 13). There were significantly lower levels of these antibodies (with the exception of IgM) in the MIF^{-/-} than in the MIF^{+/+} mice. The observation that MIF^{-/-} mice showed a significant impairment in the generation of an IgE response prompted the further examination of these mice for a lung-specific, T_{H}2 inflammatory response leading to asthma.

MIF Mice Show Decreased Airway Hyper-Responsiveness, Peri-Bronchial Infiltration, and Mucus Hyper-Production.

OVA-primed mice were subjected to aerosol challenge and measured airway reactivity in response to methacoline administration. OVA-challenge increased airway hyper-responsiveness in both the MIF^{+/+} and the MIF^{-/-} mice when compared to the control, PBS-challenged mice (FIG. 14). Airway responsiveness to methacholine (50 mg/ml) was reduced in the MIF^{-/-} mice. Lung tissues from five mice in each group then were stained with PAS and examined histologically. Lungs obtained from the control, PBS-challenged mice (both MIF^{+/+} and MIF^{-/-}) showed normal histology, while the lungs of the OVA-challenged MIF^{+/+} mice showed peri-bronchial inflammation, mucous hyperproduction, and goblet cell hypertrophy. By comparison, peri-bronchial, cellular infiltration in response to OVA-challenge was much reduced in the MIF^{-/-} mice. These functional airway and histologic data, obtained in a standard model of airway hyper-responsiveness, support a role for MIF in the development of pulmonary inflammation leading to an asthma phenotype.

Differential Infiltration of Inflammatory Cells into the Lungs of MIF^{+/+} versus MIF^{-/-} Mice.

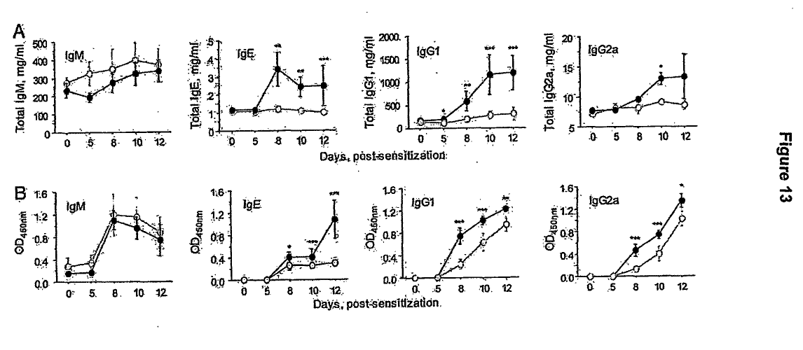

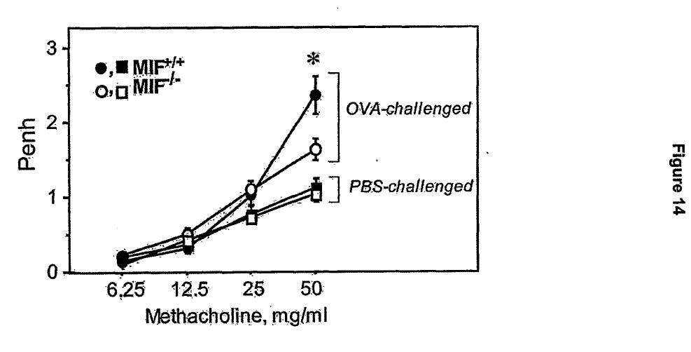

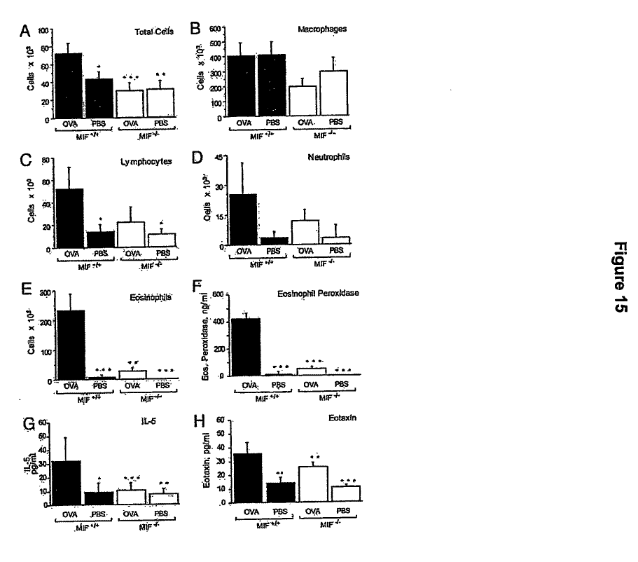

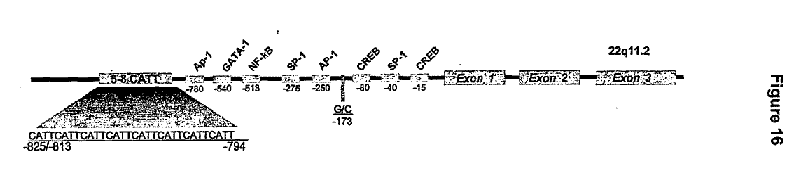

BALF analysis of the different experimental groups showed that total cell numbers were reduced in the setting of genetic ME deficiency, irrespective of OVA challenge (FIG. 15). The approximately two-fold reduction in total and mononuclear cell numbers in BALF supported the observations in lung tissue, which were further verified by enumeration of cells in high-power fields of lung tissue sections (n=5) from each experimental group. Leukocyte sub-fractionation revealed lower numbers of macrophages, lymphocytes, neutrophils, and eosinophils of MIF^{-/-} mice when compared to MIF^{+/+} mice; however this result was significant only for the eosinophil subpopulation. Because cytocentrifugation analysis may underestimate some leukocyte sub-populations (Fleury-Feith et al. (1987) Acta Cytol 31:606-610); BALF eosinophil numbers were verified by measuring eosinophil-specific peroxidase. Eosinophil peroxidase activity was reduced in the MIF^{-/-} mice, and these levels mirrored eosinophil numbers in BALF. Neither bone marrow nor circulating eosinophil numbers were found to be influenced by genetic MIF deficiency, which is agreement with the hematologic characterization of these mice (Bozza et al. (1999) J Exp Med 189:341-346 and Xie (2002) PhD Thesis, The Picower Institute for Medical Research).

The concentrations of IL-5, which mediates eosinophil activation and recruitment, and eotaxin, which is a potent, eosinophil-selective chemokine were also measured in BALF. Both IL-5 and eotaxin levels were significantly lower in the MIF^{-/-} mice than in the MIF^{+/+} mice.

Cytokine Expression in the Lungs of OVA-Challenged Mice is Reduced in MIF-Deficient Mice.

An OVA-induced increase in MIF mRNA expression was observed in wild-type mice. Previously reported increases in the mRNA levels were confirmed for the T_{H}2 cytokines, IL-4, IL-5, IL-10, and IL-13 (Herrick et al. (2003) Nature Reviews Immunology 3:405-412; Koike et al. (2003) Proc Natl Acad Sci USA 278:15685-15692; and Shibata et al. (2002) J Immunol 169:2134-40). The mRNA levels for these T_{H}2 cytokines did not differ between the MIF^{-/-} and MIF^{+/+} mice under control conditions, but the increase in mRNA levels for IL-4, IL-5, and IL-13 in response to OVA-challenge was less for the MIF^{-/-} than the wild-type mice. The mRNA levels for IL-10, or for IFN-γ, were not markedly different between the MIF^{-/-} and MIF^{+/+} mice. The small increase in IFN-γ mRNA in the MIF^{-/-} mice was not reflected by a significant change in the levels of IFN-γ protein.

The production of MIF protein was confirmed in the lungs of wild-type mice after aerosolized OVA-challenge. A significant decrease in IL-4, IL-5, IL-13, and eotaxin production was observed in MIF^{-/-} versus MIF^{+/+} mice. The concentrations of IFN-γ and IL-10 protein also were not significantly different in the absence of MIF, which is consistent with the mRNA data. Taken together, these data support a reduction in the expression and production of T_{H}2-cytokines that mediate allergic inflammation in mice deficient in MIF.

OVA-Specific, T cell Activation is Reduced in MIF^{-/-} Mice.

The splenic T cell response to OVA was significantly attenuated in the MIF^{-/-} mice, as was the production of IL-2. T cell MIF production in response to antigen stimulation in wild-type mice was rapid, peaking at 24 hrs, and preceding IL-2 production. Purified B cells proliferated equally well to OVA stimulation whether the cells were from wild-type, or MIF^{-/-} mice, suggesting that there is no intrinsic defect in B cell responses in this model of asthma.

It was examined whether the reduction in T cell proliferative responses to OVA stimulation in the setting of MIF deficiency could be attributed to an impairment in antigen-presentation. For this purpose, the well-characterized, OVA-TCR transgenic (DO11.10H2dTg) mouse strain was utilized. CD4⁺ T cells were isolated from the spleens of DO11.10H2dTg mice and co-cultured with antigen presenting cells (APCs) isolated from OVA-primed and inhalation-challenged, MIF^{-/-} or MIF^{+/+} mice. There was a significant reduction in the proliferative response of OVA-specific, T cells stimulated with MIF^{-/-} APCs when compared to MIF^{+/+} APCs, suggesting a role for APC-derived MIF in the adaptive, T cell response.

OVA-Specific, T_{H}2 Cell Responses are Reduced in MIF^{-/-} Mice.

Splenic T cells were obtained from OVA-sensitized, and PBS or OVA-challenged mice, and re-stimulated with OVA for 72 hrs for the measurement of T cell polarization responses. The intracellular production of IFN-γ (T_{H}1 response) and IL-4 (T_{H}2 response) was then measured in the CD4⁺ T cell population segregated electronically by flow cytometry. CD4⁺ T cells from the OVA-sensitized, wild-type mice showed an enhanced IL-4 response and a poor IFN-γ response, which is in accord with this disease model (Wills-Karp (1999) Annu Rev Immunol 17:255-81). The increase in IL-4 production was evident in the OVA-re-stimulated, CD4⁺ T cells irrespective of prior challenge with OVA or PBS in vivo. By contrast, the CD4⁺ T cells from the OVA-sensitized, MIF^{-/-} mice showed a markedly reduced IL-4 response and no significant induction in IFN-γ production.

Culture supernatants were analyzed for T_{H}1 and T_{H}2 cytokine production. Splenic T cells obtained from MIF^{-/-} mice and stimulated with OVA showed a marked reduction in the secretion of the T_{H}2 cytokines, 1′-4, IL-5, IL-10, IL-13, when compared to T cells obtained from their MIF^{+/+} counterparts. IFN-γ secretion by MIF^{-/-} T cells also appeared reduced, but it was not significantly different from the low levels observed in the MIF^{+/+} T cells. These data support a reduced T_{H}2 cell response in MIF^{-/-} mice in response to OVA sensitization and inhalation challenge in vivo.

Polymorphisms in the Human MIF Gene and their Relationship to Asthma Incidence and Severity.

Functional promoter polymorphisms in human MIF have been identified. These polymorphisms include a tetranucleotide sequence, CATT, that is repeated between 5-8 times at position −794 in the gene promoter (FIG. 16). An increase in CATT repeat number produces a corresponding increase in MIF promoter activity (Baugh et al. (2002) Genes Immun. 3:170-176), and SNP mapping in the promoter region further supports a role for promoter polymorphisms in regulating MIF production in vivo (De Benedetti et al. (2003) Arthritis & Rheum 48:1398-1407). Individuals with the 5-CATT (low repeat) MIF allele may be considered low MIF “expressors”, and those bearing non-5-CATT repeats (6-CATT, 7-CATT, or 8-CATT) are high MIF expressors.

To obtain an initial assessment of the role of MIF genotype in human asthma, 315 DNA samples (151 asthma, 164 controls) were collected from an ethnically-homogenous (Caucasian), adult population in Ireland. The prevalence of the different forms of the MIF CATT polymorphism in the asthmatic and control population is summarized in Table 11. Initial statistical analysis revealed no significant differences between the distribution of these alleles in the asthmatic versus the control groups (p=0.42), which is in agreement with a recently published study in a Japanese cohort (Hizawa et al. (2004) American Journal of Respiratory & Critical Care Medicine 169:1014-8). Clinical asthma severity was not examined in that study, however. The asthmatic group was then stratified by disease severity, as defined by GINA criteria (Global Initiative for Asthma) (Global Initiative for Asthma (GINA). Global Strategy for Asthma management & Prevention. 2002. NHLBI/WHO Workshop report, Bethesda, Md., National Heart, Lung & Blood Institute for Health). Significant associations were observed between the patient group encoding the MIF 5-CATT polymorphism and less severe clinical disease (5/5 vs X/X alleles, p=0.007; and 5/5+5/X versus X/X alleles, p=0.05; where 5=5-CATT and X=6-, 7-, or 8-CATT alleles). A trend was observed between the 5/5 and the combined 5/5+5/X groups for both a) higher recorded FEV₁ (p=0.06 and p=0.13 respectively) and b) fewer hospital admissions for asthma (p=0.09 and p=0.08 respectively). There was no significant association between low expression MIF alleles and oral corticosteroid usage in the preceding 12 months (p=0.95 and p=0.89 respectively). Details were also collected on the prevalence of atopy, which was present in 44% of the studied individuals. No evidence was found for any association between genotype and atopy (p=0.76). These data, taken together, support a protective role for the low-expression, 5-CATT MIF allele in the development of severe asthma.

TABLE 11
Prevalence of MIF CATT polymorphism in the enrolled asthma and
control populations

| | MIF genotype | | | | | | | | | |
|---|---|---|---|---|---|---|---|---|---|---|
| | | | | | | | | 5/5 + 5/X | X/X | |
| | 5/5 | 5/6 | 5/7 | 6/6 | 6/7 | 7/7 | 7/8 | (%) | (%) | N |
| Asthma | 15 | 46 | 9 | 57 | 21 | 2 | 1 | 70 | 81 | 151 |
| | | | | | | | | (46.4) | (53.6) | |
| Controls | 7 | 51 | 13 | 69 | 20 | 3 | 1 | 71 | 93 | 164 |
| | | | | | | | | (43.4) | (56.7) | |

5,5-CATT MIF allele;
X, 6-, 7-, or 8-CATT MIF allele;
N, number of cases.

TABLE 12
Breakdown of asthma cases by GINA criteria and distribution
of the 5-CATT (5) versus non-5-CATT MIF alleles (X)

| | | MIF genotype | | |
|---|---|---|---|---|
| GINA criteria | Asthma cases, % | 5/5, % | 5/X, % | X/X, % |
| Severe persistent | 24 | 2 | 6 | 16 |
| Moderate persistent | 31 | 1 | 11 | 19 |
| Mild persistent | 38 | 4 | 17 | 17 |
| Intermittent | 7 | 3 | 2 | 2 |

In summary, the present study assigns an important function for MIF in the immunopathogenesis of asthma via the promotion of T_{H}2 responses. The human genetic data suggest that different MIF promoter alleles, which are prevalent in the population and may exist in a balanced polymorphism (Gregersen et al. (2003) Arthritis & Rheum. 48:1171-1176), play a role in asthma clinical severity. MIF inhibition in asthma may be therapeutically beneficial, and specific intervention may be guided by the MIF genotype of affected individuals.

Materials and Methods

Mice. Mice were from Jackson Labs or bred at the Yale Animal Resources Center. MIF⁻ mice (Fingerle-Rowson et al. (2003) Proc. Natl. Acad. Sci. USA 100:9354-9359; Bozza et al. (1999) J. Exp. Med. 189:341-346) in the BALB/c genetic background were used at generation N8. BALB/c mice transgenic for the T cell receptor recognizing ovalbumin (OVA) residues 323-339 (DO11.10H2dTg) were provided by L. Cohn (Whittaker et al. (2002) American Journal of Respiratory Cell and Molecular Biology 27:593-602). Studied mice were age-matched females (6-9 wks of age) and maintained on OVA-free diets in a pathogen-free environment.

Cytokines and Antibodies. Recombinant mouse MIF was prepared and purified free of endotoxin as described (Bernhagen et al. (1994) Biochemistry 33:14144-14155). Mouse IL-2, IL-4, and IL-12p70 were from R&D Systems. Anti-IL-4 (11B11), anti-IFN-γ (R4-6A2), anti-CD3e (145-2C11), anti-CD28 (37.51), FITC-conjugated (XMG1.2), PE-conjugated anti-IL-4 (11B11), and APC-conjugated anti-CD4 (GK1.5), biotinylated anti-CD4 (GK1.5), biotinylated anti-B220 (RA3-6B2), biotinylated anti-Thy1.2 (30-H12), APC-conjugated anti-CD4 (GK1.5), PE-Cy5-conjugated anti-B220 (RA3-6B2), PE-Cy5-conjugated anti-CD8 (Ly-2), and streptavidin-PE-Cy5, were from eBioscience.

Sensitization and Challenge with OVA. Mice were sensitized with an i.p. injection of OVA (20 μg, low endotoxin) in aluminum hydroxide gel and PBS on days 0 and 5. On days 12, 13 and 14, mice inhaled aerosolized OVA or PBS for 40 mins in a chamber connected to a NE-U07 nebulizer (OMURON Co.). OVA or PBS-challenged MIF^{+/+} and MIF^{-/-} mice were sacrificed 16 hrs after the last challenge.

Measurement of Immunoglobulins. Immunoglobulin subtypes and isotypes were measured by specific ELISA (Bethyl Laboratories). OVA-specific IgE, IgG1, and IgG2a, were measured in OVA-coate microtiter plates and revealed with HRP-conjugated, anti-IgE, IgG1, or IgG2a antibodies (Bethyl Laboratories). OVA-specific IgE, IgG1, and IgG2a levels were expressed as the OD at 450 nm.

Airway Measurements. Airway hyper-responsiveness was assessed by methacoline-induced airflow obstruction of conscious mice placed in a plethysmograph (Zhu et al. (1999) Journal of Clinical Investigation 103:779-880). Mice were exposed to PBS (3 mins) and to increasing concentrations of methacholine via a NE-U07 nebulizer. Airflow obstruction was monitored for 3 mins after challenge and the Penh values were measured and averaged (Zhu et al. (1999) Journal of Clinical Investigation 103:779-88).

Bronchoalveolar Lavage Fluid (BALF) and Histologic Analysis. Lungs were lavaged with ice-cold PBS 16 hrs after the last challenge, and the BALF pooled for analysis. One half of the BALF cells were cyto-centrifuged and stained with May-Grunwald-Giemsa solution. Cells were classified as macrophages, lymphocytes, neutrophils, and eosinophils by morphological criteria. Eosinophils were quantitated by eosinophil peroxidase (Hisada et al. (1999) American Journal of Respiratory Cell and Molecular Biology 20:992-1000). Lungs were removed 16 hrs after the last challenge, inflated, and fixed overnight prior to paraffin-embedding and staining with periodic acid-Shiff (PAS).

mRNA Analysis. Total RNA was isolated using Trizol (GIBCO BRL). cDNA was prepared and amplified by SuperScript™ One-Step RT-PCR using Platinum Taq polymerase (Invitrogen) and specific primers for MIF, IL-4, IL-5, IL-10, IL-13, IFN-γ, and β-actin (MIF: 5′-ACGACATGAACGCTGCCAAC-3′ (SEQ ID NO: 29) and 5′-ACCGTGGTCTCTTATAAACC-3′ (SEQ ID NO: 30), IL-4: 5′-TATTGATGGGTCTCAACCCC-3′ (SEQ ID NO: 31) and 5′-AAGTTAAAGCATGGTGGCTCA-3′ (SEQ ID NO: 32), IL-5: 5′-AGCAATGAGACGATGAGGCT-3′ (SEQ ID NO: 33) and 5′-CATTTGCACAGTTTTGTGGG-3′ (SEQ ID NO: 34), IL-10: 5′-TGCTATGCTGCCTGCTCTTA-3′ (SEQ ID NO: 35) and 5′-TCATTTCCGATAAGGCTTGG-3′ (SEQ ID NO: 36), and 5′-TCCACAGGATCCGTGTTTTAGC-3′ (SEQ ID NO: 37), IL-13: 5′-AGACCAGACTCCCCTGTGCA-3′ (SEQ ID NO: 38) and 5′-TGGGTCCTGTAGATGGCATTG-3′ (SEQ ID NO: 39), IFN-γ: 5-TTTGAGGTCAACAACCCACA-3′ (SEQ ID NO: 40) and 5′-CGCAATCACAGTCTTGGCTA-3′ (SEQ ID NO: 41), β-actin: 5′-GGTACCACCATGTACCCAGG-3′ (SEQ ID NO: 42) and 5′-ACATCTGCTGGAAGGTGGAC-3′ (SEQ ID NO: 43)). Cycle conditions were: 30 mins at 48° C., 10 mins at 94° C., and each cycle (MIR 28 cycles, IL-4: 38 cycles, IL-5: 33 cycles, IL-10: 40 cycles, IL-13: 38 cycles, IFN-γ: 36 cycles, β-actin: 25 cycles) with 10 sec at 94° C., 45 sec at 60° C., and 1 min at 72° C. and 10 mins at 72° C. The RT-PCR of all samples from individual experiments were done in the same reaction and run on the same agarose gel.

Proliferation Assays. Spleen cells were cultured with or without OVA for 3 days (6×10⁵ cells/well). DNA synthesis was assessed by [³H]-thymidine incorporation for the last 18 hrs of culture. CD4⁺ T cells or splenic B cells were purified by positive selection using anti-CD4 mAb or anti-B220 mAb and MACS separation columns (Miltenyi Biotec). T-cell depleted splenocytes as APCs were prepared from spleen cells by negative selection using anti-Thy1.2 and streptavidin microbeads and treated with mitomycin C. Isolated CD4⁺ T cells, B cells, and APCs were >95% CD4⁺, >95% B220⁺, and <5% Thy1.2⁺, respectively. B cell studies were performed at 2×10⁵ cells/well. CD4⁺ T cells from DO11.10H2dTg mice were co-cultured with APCs for 3 days (1×10⁵/well CD4+ T cells plus 3×10⁵ APCs/well) prior to the addition of [³H]-thymidine.

Cytokine Assays. IL-4, IL-5, IL-10, and IFN-γ were assayed with BD Biosciences ELISAs, and IL-2, eotaxin, and IL-13 were measured with kits from R&D Systems. The MIF ELISA followed a previously reported capture method employing an anti-MIF IgG polyclonal antibody (detection limit: 0.16 ng/ml) (Mizue et al. (2000) International Journal of Molecular Medicine 5:397-403). Intracellular staining of IL-4 and IFN-γ was performed by using Fixation/Permeabilization Solution Kit (BD Biosciences). For the last 6 hrs, 1 μl of GolgiPlug was added to the each well and the cells incubated with APC-conjugated anti-CD4 (GK1.5), fixed, and then incubated with FITC-conjugated anti-MN-γ (XMG1.2) and PE-conjugated anti-IL-4 (11B11). Flow cytometry analysis was performed on a FACSCalibur, and the results analyzed with FlowJo software.

Patient Selection. Blood samples were collected from adults with asthma (n=151) attending a hospital outpatient department in Dublin, Ireland. Samples also were obtained from disease-free controls (n=164) matched for ethnicity (Caucassian). All patients were initially assessed and diagnosed by a single physician (S.J.L.) according to the British Thoracic Society (BTS) (The BTS/SIGN British Guideline on the Management of Asthma (2003) Thorax 58 (supplement 1), i1-i16). All patients had pulmonary function studies performed where airflow obstruction and significant reversibility were confirmed. Asthma severity was defined by the Global Initiative for Asthma (GINA) criteria (mild intermittent, mild persistent, moderate persistent and severe persistent) based on clinical and lung function assessments (Global Initiative for Asthma (GINA). Global Strategy for Asthma management & Prevention. 2002. NHLBI/WHO Workshop report, Bethesda, Md., National Heart, Lung & Blood Institute for Health). Additional criteria pertaining to asthma severity that were studied included: a) lowest FEV₁ recorded in case notes, b) number of hospital admissions for asthma, c) number of days on oral corticosteroids in prior 12 months. The control group had similar pulmonary function studies performed and no evidence of airflow obstruction was found. Atopy was defined as a positive response to an allergen panel (Hizawa et al. (2004) American Journal of Respiratory & Critical Care Medicine 169:1014-8). All studies were approved by institutional medical ethics committees.

Genotype Analysis. Genomic DNA was isolated with the QIAamp DNA Blood Mini Kits (QIAGEN Ltd.). Analysis for the CATT MIF polymorphism was performed as previously described (Baugh et al. (2002) Genes Immun. 3:170-176). Statistical analysis was performed using the open-source R-package (R Development Core Team, 2004). The associations between categorical variables were explored using tabulations, and analysed using the Chi² test and log-linear modelling (R Development Core Team. R: Language and environment for statistical computing. R Foundation for statistical computing. (ISDN: 3-900051-00-3). 2004. Vienna, Austria).

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

1.-93. (canceled)

94. A method of genotyping a subject for the presence of a polymorphism associated with MIF expression, the method comprising detecting or measuring the presence of guanine (G) or cytosine (C) at position −173 of the MIF promoter, wherein C at position −173 is associated with high MIF expression and G at position −173 is associated with low MIF expression.

95. The method of claim 94 wherein when the genotype is C at position −173, further comprises selecting said subject for treatment with a MIF antagonist.

96. The method of claim 94 wherein when the genotype is G at position −173, further comprises selecting said subject for treatment with a MIF agonist.

97. The method of claim 94 wherein C at position −173 is associated with a disease caused by a protozoan.

98. The method of claim 94, wherein C at position −173 is associated with malaria.

99. The method of claim 94, wherein C at position −173 is associated with anemia of chronic disease.

100. The method of claim 94, wherein G at position −173 is associated with an infection.

101. The method of claim 100, wherein the infection leads to respiratory disease.

102. The method of claim 94, wherein G at position −173 is associated with pneumonia.

103. The method of claim 94, wherein G at position −173 is associated with Community Acquired Pneumonia (CAP).

104. The method of claim 94, wherein G at position −173 is associated with meningitis.

105. The method of claim 94, wherein G at position −173 is associated with influenza infection.

106. The method of claim 94, wherein G at position −173 is associated with sepsis.

107. The method of claim 94 wherein when the genotype is C at position −173, further comprises identifying a subject at risk of developing a disease associated with high MIF expression.

108. The method of claim 94 wherein when the genotype is G at position −173, further comprises identifying a subject at risk of developing a disease associated with low MIF expression.

109. The method of claim 94 wherein when the genotype is C at position −173, further comprises predicting the severity of a disease associated with high MIF expression in said subject.

110. The method of claim 94 wherein when the genotype is G at position −173, further comprises predicting the severity of a disease associated with low MIF expression in said subject.

111. The method of claim 94 wherein when the genotype is C at position −173, further comprises predicting whether said subject is susceptible to a disease associated with high MIF expression.

112. The method of claim 94 wherein when the genotype is G at position −173, further comprises identifying a subject at risk of developing a disease associated with low MIF expression.

113. The method of claim 94, wherein genotyping the subject for the presence of a polymorphism associated with MIF expression comprises:
(a) contacting a sample obtained from the subject with a polynucleotide probe that hybridizes specifically to guanine in the −173 region of the MIF promoter or cytosine in the −173 region of the MIF promoter; and
(b) determining whether hybridization occurs,
wherein hybridization indicates whether the subject comprises a polymorphism associated with high MIF expression or a polymorphism associated with low MIF expression, thereby genotyping the subject for the presence of a polymorphism associated with MIF expression.

114. The method of claim 113, wherein the method further comprises:
(c) contacting the sample with a control polynucleotide probe, wherein the control polynucleotide probe does not hybridize specifically to a polymorphism associated with MIF expression, and
wherein hybridization of the polynucleotide probe but not the control polynucleotide probe indicates the presence of a MIF polymorphism associated with MIF expression.

115. The method of claim 94, wherein genotyping the subject for the presence of a polymorphism associated with MIF expression comprises:
(a) contacting a sample obtained from the subject with a pair of amplifications primers, wherein said primers are capable of amplifying a portion of the MIF promoter comprising a polymorphism associated with MIF expression;
(b) amplifying DNA in the sample, thereby producing amplified DNA;
(c) determining whether the amplified DNA comprises a guanine in the −173 region of the MIF promoter or a cytosine in the −173 region of the MIF promoter.

116. The method of claim 115, wherein the determining step comprises sequencing the amplified DNA.

117. A solid substrate for simultaneously genotyping a microsatellite repeat and a SNP, comprising at least two polynucleotide probes that are complementary to one or more polymorphic regions of the MIF gene wherein at least one of the probes detects a microsatellite repeat and at least one of the probes detects a SNP, wherein at least one of the probes hybridizes specifically to a guanine or a cytosine in the −173 region of the MIF promoter.

118. The solid substrate of claim 117, wherein at least one of the probes comprises a sequence selected from the group consisting of SEQ ID NO: 1, SEQ ID NO: 2, SEQ ID NO: 3 and SEQ ID NO: 4.

119. The solid substrate of claim 117, wherein the solid substrate comprises:
(a) a probe hybridizing specifically to SEQ ID NO: 1;
(b) a probe hybridizing specifically to SEQ ID NO: 2;
(c) a probe hybridizing specifically to SEQ ID NO: 3;
(d) a probe hybridizing specifically to SEQ ID NO: 4;
(e) a probe hybridizing specifically to guanine in the −173 region of the MIF promoter; and
(f) a probe hybridizing specifically to cytosine in the −173 region of the MIF promoter.

120. The solid substrate of claim 117, wherein the solid substrate is a chip or a microarray.

121. A method of treating anemia of chromic disease comprising administering to a subject in need thereof a therapeutically effective amount of a MIF antagonist.

122. The method of claim 121, wherein the subject is not responsive to erythropoietin (EPO) prior to the administration of the MIF antagonist.

123. The method of claim 121, further comprising administering to the subject one or more other agents that stimulate erythropoiesis.

124. The method of claim 121, further comprising administering EPO to the subject.

125. The method of claim 121, further comprising administering a TNFα antagonist or an IFNγ antagonist to the subject.

126. The method of claim 121, wherein the anemia of chronic disease results from a condition selected from the group consisting of: a pathogenic infection, cancer, an autoimmune disease or disorder, a kidney disease or disorder, organ transplant rejection and aging.

127. The method of claim 121, wherein the anemia of chronic disease results from malaria infection.

128. A method of treating malaria comprising administering to a subject in need thereof a therapeutically effective amount of a MIF antagonist.

129. A method of treating an infection comprising administering to a subject in need thereof a therapeutically effective amount of a MIF agonist.

130. The method of claim 129, wherein the infection leads to a respiratory disease.

131. The method of claim 129, wherein the subject has pneumonia.

132. The method of claim 129, wherein the subject has CAP.

133. The method of claim 129, wherein the subject has meningitis.

134. The method of claim 129, wherein the subject has influenza.

135. The method of claim 129, wherein the subject has sepsis.

136. The method of claim 129, wherein the infection is a viral infection.

137. The method of claim 129, wherein the infection is a retroviral infection.

138. A method of attenuating the biological function of MIF, comprising the use of an agent that inhibits the interaction between CD44 and CD74.

139. The method of claim 138, wherein the agent is selected from the group consisting of: a fragment of CD44, an extracellular fragment of CD44, an agent that binds CD44, an antibody or fragment thereof that binds to CD44, a small molecule, a small molecule mimic of chondroitin sulfate, heparin and a macromolecular mimic of chondroitin sulphate.

140. A method of attenuating the biological function of MIF, comprising the use of an agent that inhibits the expression of CD44.

141. The method of claim 140, wherein the agent is an siRNA or antisense polynucleotide that targets CD44.

142. A method of increasing the biological function of MIF, comprising the use of an agent that increases the interaction between MIF, CD44 and CD74.

143. A method of increasing the biological function of MIF, comprising the use of an agent that increases the interaction between CD44 and CD74.

144. A method of identifying potential agonists or antagonists of MIF, comprising:
(a) contacting a CD44 polypeptide, or a portion thereof, with a CD74 polypeptide, or portion thereof, in the presence and absence of a candidate agent; and
(b) comparing the interaction of the CD44 and CD74 polypeptides in the presence of said candidate agent with the interaction in the absence of said candidate agent,
wherein a candidate agent that enhances the interaction of the CD44 polypeptide and the CD74 polypeptide is identified as a potential agonist of MIF, and a candidate agent that inhibits the interaction of the CD44 polypeptide and the CD74 polypeptide is identified as a potential antagonist of MIF.

145. A method of identifying potential agonists or antagonists of MIF, comprising:
(a) contacting a CD44 polypeptide or a portion thereof, with a MIF polypeptide or a portion thereof and a CD74 polypeptide or a portion thereof, in the presence and absence of a candidate agent; and
(b) comparing the interaction of the CD44 polypeptide and the MIF and CD74 polypeptides in the presence of said candidate agent with the interaction in the absence of said candidate agent,
wherein a candidate agent that enhances the interaction of the CD44 polypeptide and the MIF and CD74 polypeptides is identified as a potential agonist of MIF, and a candidate agent that inhibits the interaction of CD44 polypeptide and the MIF and CD74 polypeptides is identified as a potential antagonist of MIF.

146. A kit comprising:
(a) at least one container means comprising a reagent for genotyping a subject for the presence of a polymorphism associated with high or low MIF expression, wherein said genotyping reagent is a polynucleotide probe, a polynucleotide primer, or a solid substrate that is capable of detecting a polymorphism associated with high or low MIF expression; and,
(b) a label or instructions for use of the kit.

147. A kit comprising:
(a) at least one container means comprising a premeasured dose of one or more MIF antagonists or MIF agonists; and,
(b) a label or instructions for use of the kit.
